SEQUENCE LISTING (118 sequences)

SEQ ID NO: 1 - DNA, 1746 bp, Adenovirus type 5
  CDS (1)...(1746)
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 2 - protein, 580 aa, Adenovirus type 5
KRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 3 - DNA, 1746 bp, Artificial Sequence
  5F KO1
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccagaggctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 4 - protein, 581 aa, Artificial Sequence
  5F KO1
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPEANCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 5 - DNA, 1776 bp, Artificial Sequence
  5F KO1RGD
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggtgatcattgtgattgtcgtggtgattgtttttgtacaactccaagtgcatactctatgtcattttcatgggactggtctggccacaactacattaatgaaatatttgccacatcctcttacactttttcatacattgcccaagaataa

SEQ ID NO: 6 - protein, 582 aa, Artificial Sequence
  5F KO1RGD
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDHCDCRGDCFCTTPSAYSMSFSWDWSGHNYINEIFATSS

SEQ ID NO: 7 - DNA, 1746 bp, Artificial Sequence
  5F KO12
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgttcactaaatggaggcggagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 8 - protein, 581 aa, Artificial Sequence
  5F KO12
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCSLNGGGDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 9 - DNA, 1686 bp, Artificial Sequence
  5F S*
accggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcggagccggagcctcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 10 - protein, 561 aa, Artificial Sequence
  5F S*
TGPPTVPFLTPPFVSPNGFQESPPGVLSLRLSEPLVTSNGMLALKMGNGLSLDEAGNLTSQNVTTVSPPLGAGASNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 11 - DNA, 1776 bp, Artificial Sequence
  5F S*RGD
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcggagccggagcctcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggtgatcattgtgattgtcgtggtgattgtttttgtacaactccaagtgcatactctatgtcattttcatgggactggtctggccacaactacattaatgaaatatttgccacatcctcttacactttttcatacattgcccaagaataa

SEQ ID NO: 12 - protein, 582 aa, Artificial Sequence
  5F S*RGD
MKRARPSEDTFNPVYPYDTETGPPTVPFLTPPFVSPNGFQESPPGVLSLRLSEPLVTSNGMLALKMGNGLSLDEAGNLTSQNVTTVSPPLGAGASNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDHCDCRGDCFCTTPSAYSMSFSWDWSGHNYINEIFATSS

SEQ ID NO: 13 - DNA, 1746 bp, Artificial Sequence
  5F KO1S*
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcggagccggagcctcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccagaggctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 14 - protein, 581 aa, Artificial Sequence
  5F KO1S*
MKRARPSEDTFNPVYPYDTETGPPTVPFLTPPFVSPNGFQESPPGVLSLRLSEPLVTSNGMLALKMGNGLSLDEAGNLTSQNVTTVSPPLGAGASNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPEANCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 15 - DNA, 1776 bp, Artificial Sequence
  5F KO1S*RGD
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcggagccggagcctcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccagaggctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggtgatcattgtgattgtcgtggtgattgtttttgtacaactccaagtgcatactctatgtcattttcatgggactggtctggccacaactacattaatgaaatatttgccacatcctcttacactttttcatacattgcccaagaataa

SEQ ID NO: 16 - protein, 591 aa, Artificial Sequence
  5F KO1S*RGD
MKRARPSEDTFNPVYPYDTETGPPTVPFLTPPFVSPNGFQESPPGVLSLRLSEPLVTSNGMLALKMGNGLSLDEAGNLTSQNVTTVSPPLGAGASNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPEANCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDHCDCRGDCFCTTPSAYSMSFSWDWSGHNYINEIFATSSYTFSYIAQE

SEQ ID NO: 17 - DNA, 972 bp, Artificial Sequence
  Ad35 fiber
atgaccaagagagtccggctcagtgactccttcaaccctgtctacccctatgaagatgaaagcacctcccaacacccctttataaacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacgaataa

SEQ ID NO: 18 - protein, 323 aa, Artificial Sequence
  Ad 35 fiber
MTKRVRLSDSFNPVYPYEDESTSQHPFINPGFISPNGFTQSPDGVLTLKCLTPLTTTGGSLQLKVGGGLTVDDTDGTLQENIRATAPITKNNHSVELSIGNGLETQNNKLCAKLGNGLKFNNGDICIKDSINTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDE

SEQ ID NO: 19 - DNA, 1002 bp, Artificial Sequence
  35F RGD
atgaccaagagagtccggctcagtgactccttcaaccctgtctacccctatgaagatgaaagcacctcccaacacccctttataaacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgtacattgtgattgtcgtggtgattgtttttgcgcatatgccatacaatttgaatggaatctaaatgcaagtgaatctccagaaagcaacatagctacgctgaccacatccccctttttcttttcttacattacagaagacgacgaataa

SEQ ID NO: 20 - protein, 332 aa, Artificial Sequence
  35FRGD
TKRVRLSDSFNPVYPYEDESTSQHPFINPGFISPNGFTQSPDGVLTLKCLTPLTTTGGSLQLKVGGGLTVDDTDGTLQENIRATAPITKNNHSVELSIGNGLETQNNKLCAKLGNGLKFNNGDICIKDSINTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVHCDCRGDCFCAYAIQFEWNLNASESPESNIATLTTSPFFFSYITEDDE

SEQ ID NO: 21 - DNA, 1164 bp, Artificial Sequence
  Ad41 short fiber
atgaaaagaaccagaattgaagacgacttcaaccccgtctacccctatgacaccttctcaactcccagcatcccctatgtagctccgcccttcgtttcttctgacgggttacaggaaaaacccccaggagttttagcactcaagtacactgaccccattactaccaatgctaagcatgagcttactttaaaacttggaagcaacataactttagaaaatgggttactttcggccacagttcccactgtttctcctccccttacaaacagtaacaactccctgggtttagccacatccgctcccatagctgtatcagctaactctctcacattggccaccgccgcaccactgacagtaagcaacaaccagcttagtattaacgcgggcagaggtttagttataactaacaatgccttaacagttaatcctaccggagcgctaggtttcaataacacaggagctttacaattaaatgctgcaggaggaatgagagtggacggtgccaacttaattcttcatgtagcatatccctttgaagcaatcaaccagctaacactgcgattagaaaacgggttagaagtaaccagcggaggaaagcttaacgttaagttgggatcaggcctccaatttgacagtaacggacgcattgctattagtaatagcaaccgaactcgaagtgtaccatccctcactaccatttggtctatctcgcctacgcctaactgctccatttatgaaacccaagatgcaaacctatttctttgtctaactaaaaacggagctcacgtattaggtactataacaatcaaaggtcttaaaggagcactgcgggaaatgcacgataacgctctatctttaaaacttccctttgacaatcagggaaatttacttaactgtgccttggaatcatccacctggcgttaccaggaaaccaacgcagtggcctctaatgccttaacatttatgcccaacagtacagtgtatccacgaaacaaaaccgctcacccgggcaacatgctcatccaaatctcgcctaacatcaccttcagtgtcgtctacaacgagataaacagtgggtatgcttttacttttaaatggtcagccgaaccgggaaaaccttttcacccacctaccgctgtattttgctacataactgaagaataa

SEQ ID NO: 22 - protein, 387 aa, Artificial Sequence
  Ad41 short fiber
MKRTRIEDDFNPVYPYDTFSTPSIPYVAPPFVSSDGLQEKPPGVLALKYTDPITTNAKHELTLKLGSNITLENGLLSATVPTVSPPLTNSNNSLGLATSAPIAVSANSLTLATAAPLTVSNNQLSINAGRGLVITNNALTVNPTGALGFNNTGALQLNAAGGMRVDGANLILHVAYPFEAINQLTLRLENGLEVTSGGKLNVKLGSGLQFDSNGRIAISNSNRTRSVPSLTTIWSISPTPNCSIYETQDANLFLCLTKNGAHVLGTITIKGLKGALREMHDNALSLKLPFDNQGNLLNCALESSTWRYQETNAVASNALTFMPNSTVYPRNKTAHPGNMLIQISPNITFSVVYNEINSGYAFTFKWSAEPGKPFHPPTAVFCYITEE

SEQ ID NO: 23 - DNA, 1194 bp, Artificial Sequence
  41sF RGD
atgaaaagaaccagaattgaagacgacttcaaccccgtctacccctatgacaccttctcaactcccagcatcccctatgtagctccgcccttcgtttcttctgacgggttacaggaaaaacccccaggagttttagcactcaagtacactgaccccattactaccaatgctaagcatgagcttactttaaaacttggaagcaacataactttagaaaatgggttactttcggccacagttcccactgtttctcctccccttacaaacagtaacaactccctgggtttagccacatccgctcccatagctgtatcagctaactctctcacattggccaccgccgcaccactgacagtaagcaacaaccagcttagtattaacgcgggcagaggtttagttataactaacaatgccttaacagttaatcctaccggagcgctaggtttcaataacacaggagctttacaattaaatgctgcaggaggaatgagagtggacggtgccaacttaattcttcatgtagcatatccctttgaagcaatcaaccagctaacactgcgattagaaaacgggttagaagtaaccagcggaggaaagcttaacgttaagttgggatcaggcctccaatttgacagtaacggacgcattgctattagtaatagcaaccgaactcgaagtgtaccatccctcactaccatttggtctatctcgcctacgcctaactgctccatttatgaaacccaagatgcaaacctatttctttgtctaactaaaaacggagctcacgtattaggtactataacaatcaaaggtcttaaaggagcactgcgggaaatgcacgataacgctctatctttaaaacttccctttgacaatcagggaaatttacttaactgtgccttggaatcatccacctggcgttaccaggaaaccaacgcagtggcctctaatgccttaacatttatgcccaacagtacagtgtatccacgaaacaaaaccgctcacccgggcaacatgctcatccaaatctcgcctaacatcaccttcagtgtcgtctacaacgagataaactgtgattgtcgtggtgattgtttttgtactagtgggtatgcttttacttttaaatggtcagccgaaccgggaaaaccttttcacccacctaccgctgtattttgctacataactgaagaataa

SEQ ID NO: 24 - protein, 397 aa, Artificial Sequence
  41sFRGD
MKRTRIEDDFNPVYPYDTFSTPSIPYVAPPFVSSDGLQEKPPGVLALKYTDPITTNAKHELTLKLGSNITLENGLLSATVPTVSPPLTNSNNSLGLATSAPIAVSANSLTLATAAPLTVSNNQLSINAGRGLVITNNALTVNPTGALGFNNTGALQLNAAGGMRVDGANLILHVAYPFEAINQLTLRLENGLEVTSGGKLNVKLGSGLQFDSNGRIAISNSNRTRSVPSLTTIWSISPTPNCSIYETQDANLFLCLTKNGAHVLGTITIKGLKGALREMHDNALSLKLPFDNQGNLLNCALESSTWRYQETNAVASNALTFMPNSTVYPRNKTAHPGNMLIQISPNITFSVVYNEINCDCRGDCFCTSGYAFTFKWSAEPGKPFHPPTAVFCYITEE

SEQ ID NO: 25 - DNA, 1737 bp, Artificial Sequence
  Ad5 penton
atgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatagccgcggctacccctacgacgtgcccgactacgcgggcaccagcgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttga

SEQ ID NO: 26 - protein, 577 aa, Artificial Sequence
  Ad5 penton
RRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDSRGYPYDVPDYAGTSATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 27 - DNA, 1773 bp, Artificial Sequence
  5TS35H
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacgaataa

SEQ ID NO: 28 - protein, 590 aa, Artificial Sequence
  5TS35H
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDE

SEQ ID NO: 29 - DNA, 945 bp, Artificial Sequence
  35TS5H
atgaccaagagagtccggctcagtgactccttcaaccctgtctacccctatgaagatgaaagcacctcccaacacccctttataaacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 30 - protein, 314 aa, Artificial Sequence
  35TS5H
MTKRVRLSDSFNPVYPYEDESTSQHPFINPGFISPNGFTQSPDGVLTLKCLTPLTTTGGSLQLKVGGGLTVDDTDGTLQENIRATAPITKNNHSVELSIGNGLETQNNKLCAKLGNGLKFNNGDICIKDSINTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 31 - DNA, 1098 bp, Adenovirus type 37
  CDS (1)...(1098)
atgtcaaagaggctccgggtggaagatgacttcaaccccgtctacccctatggctacgcgcggaatcagaatatccccttcctcactcccccctttgtctcctccgatggattcaaaaacttcccccctggggtactgtcactcaaactggctgatccaatcaccattaccaatggggatgtatccctcaaggtgggaggtggtctcactttgcaagatggaagcctaactgtaaaccctaaggctccactgcaagttaatactgataaaaaacttgagcttgcatatgataatccatttgaaagtagtgctaataaacttagtttaaaagtaggacatggattaaaagtattagatgaaaaaagtgctgcggggttaaaagatttaattggcaaacttgtggttttaacaggaaaaggaataggcactgaaaatttagaaaatacagatggtagcagcagaggaattggtataaatgtaagagcaagagaagggttgacatttgacaatgatggatacttggtagcatggaacccaaagtatgacacgcgcacactttggacaacaccagacacatctccaaactgcacaattgctcaagataaggactctaaactcactttggtacttacaaagtgtggaagtcaaatattagctaatgtgtctttgattgtggtcgcaggaaagtaccacatcataaataataagacaaatccaaaaataaaaagttttactattaaactgctatttaataagaacggagtgcttttagacaactcaaatcttggaaaagcttattggaactttagaagtggaaattccaatgtttcgacagcttatgaaaaagcaattggttttatgcctaatttggtagcgtatccaaaacccagtaattctaaaaaatatgcaagagacatagtttatggaactatatatcttggtggaaaacctgatcagccagcagtcattaaaactacctttaaccaagaaactggatgtgaatactctatcacatttaactttagttggtccaaaacctatgaaaatgttgaatttgaaaccacctcttttaccttctcctatattgcccaagaatga

SEQ ID NO: 32 - protein, 365 aa, Adenovirus type 37
MSKRLRVEDDFNPVYPYGYARNQNIPFLTPPFVSSDGFKNFPPGVLSLKLADPITITNGDVSLKVGGGLTLQDGSLTVNPKAPLQVNTDKKLELAYDNPFESSANKLSLKVGHGLKVLDEKSAAGLKDLIGKLVVLTGKGIGTENLENTDGSSRGIGINVRAREGLTFDNDGYLVAWNPKYDTRTLWTTPDTSPNCTIAQDKDSKLTLVLTKCGSQILANVSLIVVAGKYHIINNKTNPKIKSFTIKLLFNKNGVLLDNSNLGKAYWNFRSGNSNVSTAYEKAIGFMPNLVAYPKPSNSKKYARDIVYGTIYLGGKPDQPAVIKTTFNQETGCEYSITFNFSWSKTYENVEFETTSFTFSYIAQE

SEQ ID NO: 33 - DNA, 1098 bp, Adenovirus type 19p
  CDS (1)...(1098)
atgtcaaagaggctccgggtggaagatgacttcaaccccgtctacccctatggctacgcgcggaatcagaatatccccttcctcactcccccctttgtctcctccgatggattcaaaaacttcccccctggggtactgtcactcaaactggctgatccaatcaccattaccaatggggatgtatccctcaaggtgggaggtggtctcactttgcaagatggaagcctaactgtaaaccctaaggctccactgcaagttactactgataaaaaacttgagcttgcatatgataatccatttgaatgtagtgctaataaatttagtttaaaagtaggacatggattaaaagtattagatgaaaaaagtgctgcggggttaaaagatttaattggcaaacttgtggttttaacaggaaaaggaataggcactgaaaatttagaaaatacagatggtagcagcagaggaattggtataaatgtaagagcaagagaagggttgacatttgacaatgatggatacttggtagcatggaacccaaagtatgacacgcgcacactttggacaacaccagacacatctccaaactgcacaattgctcaggataaggactctaaactcactttggtacttacaaagtgtggaagtcaaatattagctaatgtgtctttgattgtggtcgcaggaaagtaccacatcataaataataagacaaatccagaaataaaaagttttactattaaactgttatttaataagaacggagtgcttttagacaactcaaatcttggaaaagcttattggaactttagaagtggaaattccaatgtttcgacagcttatgaaaaagcaattggttttatgcctaatttagtagcgtatccaaaacccagtaattctaaaaaatatgcaagagacatagtttatggaactatatatcttggtggaaaacctgatcagccagcagtcattaaaactacctttaaccaagaaactggatgtgaatactctatcacatttgactttagttggtccaaaacctatgaaaatgttgaatttgaaaccacctcttttaccttctcctatattgcccaagaatga

SEQ ID NO: 34 - protein, 365 aa, Adenovirus type 19p
MSKRLRVEDDFNPVYPYGYARNQNIPFLTPPFVSSDGFKNFPPGVLSLKLADPITITNGDVSLKVGGGLTLQDGSLTVNPKAPLQVTTDKKLELAYDNPFECSANKFSLKVGHGLKVLDEKSAAGLKDLIGKLVVLTGKGIGTENLENTDGSSRGIGINVRAREGLTFDNDGYLVAWNPKYDTRTLWTTPDTSPNCTIAQDKDSKLTLVLTKCGSQILANVSLIVVAGKYHIINNKTNPEIKSFTIKLLFNKNGVLLDNSNLGKAYWNFRSGNSNVSTAYEKAIGFMPNLVAYPKPSNSKKYARDIVYGTIYLGGKPDQPAVIKTTFNQETGCEYSITFDFSWSKTYENVEFETTSFTFSYIAQE

SEQ ID NO: 35 - DNA, 1116 bp, Adenovirus type 30
  CDS (1)...(1116)
atgtcaaagaggctccgggtggaagatgacttcaaccccgtctacccctatggctacgcgcggaatcagaatatccccttccttactcccccctttgtctcatccgatggattcaaaaacttcccacctggggtcctgtcactcaaactggctgacccaatcgccatcactaatggggatgtctcactcaaggtgggagggggactaactgtggaacaagatagtggaaacctaagtgtaaaccctaaggctccattgcaagttggaacagacaaaaaactggaattggctttagcacctccatttgatgtcagagataacaagctagctattctagtaggagatggattaaaggtaatagatagatcaatatctgatttgccaggtttgttaaactatcttgtagttttgactggcaaaggaattggaaatgaagaattaaaaaatgacgatggtagcaataaaggagtcggtttatgtgtgagaattggagaaggaggtggtttaacttttgatgataaaggttatttagtagcatggaacaataaacatgacatccgcacactttggacaactttagacccttctccaaattgtaagatagatatagaaaaagactcaaaactaactttggtactgacaaagtgcggaagtcagattttggcaaatgtatctctaattatagtcaacggaaagttcaagatccttaataacaaaacagacccatccctacctaaatcatttaacatcaaactactgtttgatcaaaatggagttctattggaaaattcaaacattgaaaaacagtacctaaactttagaagtggagactctattcttccagagccatataaaaatgcaattggatttatgcctaatttactagcttatgctaaagctacaactgatcagtctaaaatttatgcaaggaacactatatatggaaatatctacttagataatcagccatataatccagttgtaattaaaattacttttaataatgaagcagatagtgcttattctatcacttttaactattcatggaccaaggactatgacaatatcccttttgattctacttcatttaccttctcctatatcgcccaagaatga

SEQ ID NO: 36 - protein, 370 aa, Adenovirus type 30
SKRLRVEDDFNPVYPYGYARNQNIPFLTPPFVSSDGFKNFPPGVLSLKLADPIAITNGDVSLKVGGGLTVEQDSGNLSVNPKAPLQVGTDKKLELALAPPFDVRDNKLAILVGDGLKVIDRSISDLPGLLNYLVVLTGKGIGNEELKNDDGSNKGVGLCVRIGEGGGLTFDDKGYLVAWNNKHDIRTLWTTLDPSPNCKIDIEKDSKLTLVLTKCGSQILANVSLIIVNGKFKILNNKTDPSLPKSFNIKLLFDQNGVLLENSNIEKQYLNFRSGDSILPEPYKNAIGFMPNLLAYAKATTDQSKIYARNTIYGNIYLDNQPYNPVVIKITFNNEADSAYSITFNYSWTKDYDNIPFDSTSFTFSYIAQE

SEQ ID NO: 37 - DNA, 1062 bp, Adenovirus type 16
  CDS (1)...(1062)
atggccaaacgagctcggctaagcagctccttcaatccggtctacccctatgaagatgaaagcagctcacaacacccctttataaaccctggtttcatttcctcaaatggttttgcacaaagcccagatggagttctaactcttaaatgtgttaatccactcactaccgccagcggacccctccaacttaaagttggaagcagtcttacagtagatactatcgatgggtctttggaggaaaatataactgccgcagcgccactcactaaaactaaccactccataggtttattaataggatctggcttgcaaacaaaggatgataaactttgtttatcgctgggagatgggttggtaacaaaggatgataaactatgtttatcgctgggagatgggttaataacaaaaaatgatgtactatgtgccaaactaggacatggccttgtgtttgactcttccaatgctatcaccatagaaaacaacaccttgtggacaggcgcaaaaccaagcgccaactgtgtaattaaagagggagaagattccccagactgtaagctcactttagttctagtgaagaatggaggactgataaatggatacataacattaatgggagcctcagaatatactaacaccttgtttaaaaacaatcaagttacaatcgatgtaaacctcgcatttgataatactggccaaattattacttacctatcatcccttaaaagtaacctgaactttaaagacaaccaaaacatggctactggaaccataaccagtgccaaaggcttcatgcccagcaccaccgcctatccatttataacatacgccactgagaccctaaatgaagattacatttatggagagtgttactacaaatctaccaatggaactctctttccactaaaagttactgtcacactaaacagacgtatgttagcttctggaatggcctatgctatgaatttttcatggtctctaaatgcagaggaagccccggaaactaccgaagtcactctcattacctcccccttctttttttcttatatcagagaagatgactga

SEQ ID NO: 38 - protein, 353 aa, Adenovirus type 16
MAKRARLSSSFNPVYPYEDESSSQHPFINPGFISSNGFAQSPDGVLTLKCVNPLTTASGPLQLKVGSSLTVDTIDGSLEENITAAAPLTKTNHSIGLLIGSGLQTKDDKLCLSLGDGLVTKDDKLCLSLGDGLITKNDVLCAKLGHGLVFDSSNAITIENNTLWTGAKPSANCVIKEGEDSPDCKLTLVLVKNGGLINGYITLMGASEYTNTLFKNNQVTIDVNLAFDNTGQIITYLSSLKSNLNFKDNQNMATGTITSAKGFMPSTTAYPFITYATETLNEDYIYGECYYKSTNGTLFPLKVTVTLNRRMLASGMAYAMNFSWSLNAEEAPETTEVTLITSPFFFSYIREDD

SEQ ID NO: 39 - DNA, 972 bp, Adenovirus type 35
  CDS (1)...(972)
atgaccaagagagtccggctcagtgactccttcaaccctgtctacccctatgaagatgaaagcacctcccaacacccctttataaacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaa

SEQ ID NO: 40 - protein, 323 aa, Adenovirus type 35
MTKRVRLSDSFNPVYPYEDESTSQHPFINPGFISPNGFTQSPDGVLTLKCLTPLTTTGGSLQLKVGGGLTVDDTDGTLQENIRATAPITKNNHSVELSIGNGLETQNNKLCAKLGNGLKFNNGDICIKDSINTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDN

SEQ ID NO: 41 - DNA, 1062 bp, Artificial Sequence
  Ad5/Ad16 chimeric fiber
atgaagcgcgcaagaccgtctgaagataccttcaaccccgtgtatccatatgaagatgaaagcagctcacaacacccctttataaaccctggtttcatttcctcaaatggttttgcacaaagcccagatggagttctaactcttaaatgtgttaatccactcactaccgccagcggacccctccaacttaaagttggaagcagtcttacagtagatactatcgatgggtctttggaggaaaatataactgccgcagcgccactcactaaaactaaccactccataggtttattaataggatctggcttgcaaacaaaggatgataaactttgtttatcgctgggagatgggttggtaacaaaggatgataaactatgtttatcgctgggagatgggttaataacaaaaaatgatgtactatgtgccaaactaggacatggccttgtgtttgactcttccaatgctatcaccatagaaaacaacaccttgtggacaggcgcaaaaccaagcgccaactgtgtaattaaagagggagaagattccccagactgtaagctcactttagttctagtgaagaatggaggactgataaatggatacataacattaatgggagcctcagaatatactaacaccttgtttaaaaacaatcaagttacaatcgatgtaaacctcgcatttgataatactggccaaattattacttacctatcatcccttaaaagtaacctgaactttaaagacaaccaaaacatggctactggaaccataaccagtgccaaaggcttcatgcccagcaccaccgcctatccatttataacatacgccactgagaccctaaatgaagattacatttatggagagtgttactacaaatctaccaatggaactctctttccactaaaagttactgtcacactaaacagacgtatgttagcttctggaatggcctatgctatgaatttttcatggtctctaaatgcagaggaagccccggaaactaccgaagtcactctcattacctcccccttctttttttcttatatcagagaagatgactga

SEQ ID NO: 42 - protein, 353 aa, Artificial Sequence
  Ad5/Ad16 chimeric fiber
MKRARPSEDTFNPVYPYEDESSSQHPFINPGFISSNGFAQSPDGVLTLKCVNPLTTASGPLQLKVGSSLTVDTIDGSLEENITAAAPLTKTNHSIGLLIGSGLQTKDDKLCLSLGDGLVTKDDKLCLSLGDGLITKNDVLCAKLGHGLVFDSSNAITIENNTLWTGAKPSANCVIKEGEDSPDCKLTLVLVKNGGLINGYITLMGASEYTNTLFKNNQVTIDVNLAFDNTGQIITYLSSLKSNLNFKDNQNMATGTITSAKGFMPSTTAYPFITYATETLNEDYIYGECYYKSTNGTLFPLKVTVTLNRRMLASGMAYAMNFSWSLNAEEAPETTEVTLITSPFFFSYIREDD

SEQ ID NO: 43 - DNA, 972 bp, Artificial Sequence
  Ad5/Ad35 chimeric fiber
atgaagcgcgcaagaccgtctgaagataccttcaaccccgtgtatccatatgaagatgaaagcacctcccaacacccctttataaacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaa

SEQ ID NO: 44 - protein, 323 aa, Artificial Sequence
  Ad5/Ad35 chimeric fiber
MKRARPSEDTFNPVYPYEDESTSQHPFINPGFISPNGFTQSPDGVLTLKCLTPLTTTGGSLQLKVGGGLTVDDTDGTLQENIRATAPITKNNHSVELSIGNGLETQNNKLCAKLGNGLKFNNGDICIKDSINTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDN

SEQ ID NO: 45 - protein, 4 aa, Artificial Sequence
  HSP binding motif
KKTK

SEQ ID NO: 46 - protein, 4 aa, Artificial Sequence
  conserved sequence
TLWT

SEQ ID NO: 47 - DNA, 7607 bp, Artificial Sequence
  GRE5-E1-SV40-Hygro
tctagaagatccgctgtacaggatgttctagctactttattagatccgctgtacaggatgttctagctactttattagatccgctgtacaggatgttctagctactttattagatccgctgtacaggatgttctagctactttattagatccgtgtacaggatgttctagctactttattagatcgatctcctggccgttcggggtcaaaaaccaggtttggctataaaagggggtgggggcgcgttcgtcctcactctcttccgcatcgctgtctgcgagggccaggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattagatctaagcttctgcagctcgaggactcggtcgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctcagctgactgcttaagtcgcaagccgaattggatccaattcggatcgatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattaattcgggg

SEQ ID NO: 48 - DNA, 11600 bp, Artificial Sequence
  MMTV-E2a-SV40-Neo
gaattccgcattgcagagatattgtatttaagtgcctagctcgatacaataaacgccatttgaccattcaccacattggtgtgcacctccaagcttgggcagaaatggttgaactcccgagagtgtcctacacctaggggagaagcagccaaggggttgtttcccaccaaggacgacccgtctgcgcacaaacggatgagcccatcagacaaagacatattcattctctgctgcaaacttggcatagctctgctttgcctggggctattgggggaagttgcggttcgtgctcgcagggctctcacccttgactcttttaatagctcttctgtgcaagattacaatctaaacaattcggagaactcgaccttcctcctgaggcaaggaccacagccaacttcctcttacaagccgcatcgattttgtccttcagaaatagaaataagaatgcttgctaaaaattatatttttaccaataagaccaatccaataggtagattattagttactatgttaagaaatgaatcattatcttttagtactatttttactcaaattcagaagttagaaatgggaatagaaaatagaaagagacgctcaacctcaattgaagaacaggtgcaaggactattgaccacaggcctagaagtaaaaaagggaaaaaagagtgtttttgtcaaaataggagacaggtggtggcaaccagggacttataggggaccttacatctacagaccaacagatgcccccttaccatatacaggaagatatgacttaaattgggataggtgggttacagtcaatggctataaagtgttatatagatccctcccttttcgtgaaagactcgccagagctagacctccttggtgtatgttgtctcaagaagaaaaagacgacatgaaacaacaggtacatgattatatttatctaggaacaggaatgcacttttggggaaagattttccataccaaggaggggacagtggctggactaatagaacattattctgcaaaaactcatggcatgagttattatgaatagcctttattggcccaaccttgcggttcccagggcttaagtaagtttttggttacaaactgttcttaaaacgaggatgtgagacaagtggtttcctgacttggtttggtatcaaaggttctgatctgagctctgagtgttctattttcctatgttcttttggaatttatccaaatcttatgtaaatgcttatgtaaaccaagatataaaagagtgctgattttttgagtaaacttgcaacagtcctaacattcacctcttgtgtgtttgtgtctgttcgccatcccgtctccgctcgtcacttatccttcactttccagagggtccccccgcagaccccggcgaccctcaggtcggccgactgcggcagctggcgcccgaacagggaccctcggataagtgacccttgtctctatttctactatttggtgtttgtcttgtattgtctctttcttgtctggctatcatcacaagagcggaacggactcaccatagggaccaagctagcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcgcgaccggccgtattgactatggcgcaggcgagcttgtgtggagaaacaaagcctggaaagcgcttgtcataggtgcccaaaaaatatggcccacaaccaagatctttgacaatggctttcagttcctgctcactggagcccatggcggcagctgttgttgatgttgcttgcttctttatgttgtggcgttgccggccgagaagggcgtgcgcaggtacacggtttcgatgacgccgcggtgcggccggtgcacacggaccacgtcaaagacttcaaacaaaacataaagaagggtgggctcgtccatgggatccatatatagggcccgggttataattacctcaggtcgacctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccaggaagctcctctgtgtcctcataaaccctaacctcctctacttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagctccaaaggttccccaccaacagcaaaaaaatgaaaatttgacccttgaatgggttttccagcaccattttcatgagttttttgtgtccctgaatgcaagtttaacatagcagttaccccaataacctcagttttaacagtaacagcttcccacatcaaaatatttccacaggttaagtcctcatttaaattaggcaaaggaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccact

SEQ ID NO: 49 - protein, 8 aa, Artificial Sequence
  Ad5 penton residues 337-344
HAIRGDTF

SEQ ID NO: 50 - protein, 15 aa, Artificial Sequence
  PD1 penton mutation
SRGYPYDVPDYAGTS

SEQ ID NO: 51 - DNA, 35211 bp, Artificial Sequence
  Plasmid Av1nBg
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtacgtaccagtgcactggcctaggaagcttggtaccggtgaattcgctagcgttcgcgccccgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccggccctggccactctcttccgcatcgctgtctgcgggggccagctgttgggctcgcggttgaggacaaactcttcgcggtctttccagtactcttggatcggaaacccgtcggcctccgaacggtactccgccgccgagggacctgagcgagtccgcatcgaccggatcggaaaacctctcgagaaaggcgtgtaaccagtcacagtcgctctagaactagtggatcccccgggctgcaggaattcgatctagatggataaaggtccaaaaaagaagagaaaggtagaagaccccaaggactttccttcagaattgctaagttttttgagtgattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaatttcagctgagcgccggtcgctaccattaccagttggtctggtgtcaaaaataataatctcgaatcaagcttatcgataccgtcgaaacttgtttattgcagcttataatggttacaaataaagcaatagcatcacaaatttcacaaataaagcatttttttcactgcattctagttgtggtttgtccaaactcatcaatgtatcttatcatgtctggatccgacctcggatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttcaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaattaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 52 - DNA, 33622 bp, Artificial Sequence
  Plasmid Av3nBg
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccagggcgcgccccgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccggccctggccactctcttccgcatcgctgtctgcgggggccagctgttgggctcgcggttgaggacaaactcttcgcggtctttccagtactcttggatcggaaacccgtcggcctccgaacggtactccgccgccgagggacctgagcgagtccgcatcgaccggatcggaaaacctctcgagaaaggcgtgtaaccagtcacagtcgctctagaactagtggatcccccgggctgcaggaattcgatctagatggataaaggtccaaaaaagaagagaaaggtagaagaccccaaggactttccttcagaattgctaagttttttgagtgattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaatttcagctgagcgccggtcgctaccattaccagttggtctggtgtcaaaaataataatctcgaatcaagcttatcgataccgtcgaaacttgtttattgcagcttataatggttacaaataaagcaatagcatcacaaatttcacaaataaagcatttttttcactgcattctagttgtggtttgtccaaactcatcaatgtatcttatcatgtctggatccgacctcggatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 53 - DNA, 20 bp, Artificial Sequence
  Primer 5FF
gaacaggaggtgagcttaga

SEQ ID NO: 54 - DNA, 43 bp, Artificial Sequence
  Primer 5FR
tccgcctccatttagtgaacagttaggagatggagctggtgtg

SEQ ID NO: 55 - DNA, 44 bp, Artificial Sequence
  Primer 3FF
tcactaaatggaggcggagatgctaaactcactttggtcttaac

SEQ ID NO: 56 - DNA, 20 bp, Artificial Sequence
  Primer 3FR
gtggcaggttgaatactagg

SEQ ID NO: 57 - DNA, 57 bp, Artificial Sequence
  Penton 1 Oligonucleotide
cgcggaagagaactccaacgcggcagccgcggcaatgcagccggtggaggacatgaa

SEQ ID NO: 58 - DNA, 59 bp, Artificial Sequence
  Penton 2 Oligonucleotide
tatcgttcatgtcctccaccggctgcattgccgcggctgccgcgttggagttctcttcc

SEQ ID NO: 59 - DNA, 75 bp, Artificial Sequence
  Penton 3 Oligonucleotide
cgatagccgcggctacccctacgacgtgcccgactacgcgggcaccagcgccacacgggctgaggagaagcgcgc

SEQ ID NO: 60 - DNA, 73 bp, Artificial Sequence
  Penton 4 Oligonucleotide
tcagcgcgcttctcctcagcccgtgtggcgctggtgcccgcgtagtcgggcacgtcgtaggggtagccgcggc

SEQ ID NO: 61 - DNA, 19 bp, Artificial Sequence
  Hexon Forward Primer
cttcgatgatgccgcagtg

SEQ ID NO: 62 - DNA, 19 bp, Artificial Sequence
  Hexon Reverse Primer
gggctcaggtactccgagg

SEQ ID NO: 63 - DNA, 25 bp, Artificial Sequence
  Hexon Probe
ttacatgcacatctcgggccaggac

SEQ ID NO: 64 - DNA, 40 bp, Artificial Sequence
  5HSPR primer
ggctccggctccgagaggtgggctcacagtggttacattt

SEQ ID NO: 65 - DNA, 27 bp, Artificial Sequence
  P-0005/U primer
ctctagaaatggacggaattattacag

SEQ ID NO: 66 - DNA, 32 bp, Artificial Sequence
  P-0006/L primer
tcttggtcatctgcaacaacatgaagatagtg

SEQ ID NO: 67 - DNA, 32 bp, Artificial Sequence
  P-0007/U primer
gttgttgcagatgaccaagagagtccggctca

SEQ ID NO: 68 - DNA, 73 bp, Artificial Sequence
  35FMun primer
agcaattgaaaaataaacacgttgaaacataacacaaacgattctttagttgtcgtcttctgtaatgtaagaa

SEQ ID NO: 69 - DNA, 24 bp, Artificial Sequence
  P-0009 primer
agcaattgaaaaataaacacgttg

SEQ ID NO: 70 - DNA, 20 bp, Artificial Sequence
  Primer P1
gaacaggaggtgagcttaga

SEQ ID NO: 71 - DNA, 42 bp, Artificial Sequence
  Primer P2
gttaggtggagggtttattccggtccacaaagttagcttatc

SEQ ID NO: 72 - DNA, 42 bp, Artificial Sequence
  Primer P3
gataagctaactttgtggaccggaataaaccctccacctaac

SEQ ID NO: 73 - DNA, 20 bp, Artificial Sequence
  Primer P4
gtggcaggttgaatactagg

SEQ ID NO: 74 - DNA, 41 bp, Artificial Sequence
  Primer P5
gttaggagatggagctggtgtagtccataaggtgttaatac

SEQ ID NO: 75 - DNA, 41 bp, Artificial Sequence
  Primer P6
gtattaacaccttatggactacaccagctccatctcctaac

SEQ ID NO: 76 - DNA, 54 bp, Artificial Sequence
  Primer P7
tgcgcaaaaacaatcaccacgacaatcacaatgtacattggaagaaatcatacg

SEQ ID NO: 77 - DNA, 54 bp, Artificial Sequence
  Primer P8
acattgtgattgtcgtggtgattgtttttgcgcatatgccatacaatttgaatg

SEQ ID NO: 78 - protein, 10 aa, Artificial Sequence
  cRGD peptide
HCDCRGDCFC

SEQ ID NO: 79 - DNA, 32 bp, Artificial Sequence
  P-0010/L primer
ttcttttcatctgcaacaacatgaagatagtg

SEQ ID NO: 80 - DNA, 32 bp, Artificial Sequence
  P-0011/U primer
gttgttgcagatgaaaagaaccagaattgaag

SEQ ID NO: 81 - DNA, 73 bp, Artificial Sequence
  P-0012/L primer
tgcaattgaaaaataaacacgttgaaacataacacaaacgattctttattcttcagttatgtagcaaaataca

SEQ ID NO: 82 - DNA, 56 bp, Artificial Sequence
  41sRGDR Primer
agtacaaaaacaatcaccacgacaatcacagtttatctcgttgtagacgacactga

SEQ ID NO: 83 - DNA, 51 bp, Artificial Sequence
  41sRGDF Primer
tgtgattgtcgtggtgattgtttttgtactagtgggtatgcttttactttt

SEQ ID NO: 84 - DNA, 48 bp, Artificial Sequence
  L37 Primer
tgtcttggatccaagatgaagcgcgcccgccccagcgaagatgacttc

SEQ ID NO: 85 - DNA, 28 bp, Artificial Sequence
  37FR Primer
aaacacggcggccgctctttcattcttg

SEQ ID NO: 86 - protein, 8 aa, Artificial Sequence
  Native Ad37 N-terminus
MSKRLRVE

SEQ ID NO: 87 - protein, 8 aa, Artificial Sequence
  Modified Ad37 N-terminus
MKRARPSE

SEQ ID NO: 88 - DNA, 1240 bp, Artificial Sequence
  Ad5 TPL sequence
ggatccactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacgagatccgtactccgccgccgagggacctgagcgagtccgcatcgaccggatcggaaaacctctcgagaaaggcgtctaaccagtcacagtcgcaagatct

SEQ ID NO: 89 - DNA, 22 bp, Artificial Sequence
  Primer F16 5′
ccggtctacccatatgaagatg

SEQ ID NO: 90 - DNA, 28 bp, Artificial Sequence
  Primer F16 3′
tggtgcggccgctcagtcatcttctctg

SEQ ID NO: 91 - DNA, 34 bp, Artificial Sequence
  Primer F35 3′
tggtgcggccgcttagttgtcgtcttctgtaatg

SEQ ID NO: 92 - DNA, 10837 bp, Artificial Sequence
  Plasmid p5FloxHRF
ggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaattaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatggatcagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggaattaaccctcactaaagggaacaaaagctggagctccaccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagataacttcgtataatgtatgctatacgaagttatgctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgca

SEQ ID NO: 93 - DNA, 32 bp, Artificial Sequence
  MunI TOP oligo
aattgtgttatgtttaaacgtgtttatttttg

SEQ ID NO: 94 - DNA, 32 bp, Artificial Sequence
  MunI BOTTOM oligo
aattcaaaaataaacacgtttaaacataacac

SEQ ID NO: 95 - DNA, 26 bp, Artificial Sequence
  Primer F37 5′SphI
taccaatggcatgctatccctcaagg

SEQ ID NO: 96 - DNA, 25 bp, Artificial Sequence
  Primer F37 3′EcoRI
aaacacgggaattcgtctttcattc

SEQ ID NO: 97 - DNA, 24 bp, Artificial Sequence
  Primer F16 5′SphI
gccagcggcatgctccaacttaaa

SEQ ID NO: 98 - DNA, 28 bp, Artificial Sequence
  Primer F16 3′MunI
tttatcaattgtgttgtcagtcatcttc

SEQ ID NO: 99 - DNA, 25 bp, Artificial Sequence
  Forward Primer (TPL exon 3)
ctcaacaattgtggatccgtactcc

SEQ ID NO: 100 - DNA, 25 bp, Artificial Sequence
  Reverse Primer (TPL exon 3)
gtgctcagcagatcttgcgactgtg

SEQ ID NO: 101 - DNA, 25 bp, Artificial Sequence
  Forward Primer (TPL exons 1/2)
ggcgcgttcggatccactctcttcc

SEQ ID NO: 102 - DNA, 28 bp, Artificial Sequence
  Reverse Primer (TPL exons 1/2)
ctacatgctaggcagatctcgttcggag

SEQ ID NO: 103 - DNA, 1240 bp, Artificial Sequence; identical to SEQ ID NO: 88
  TPL sequence with restriction sites

SEQ ID NO: 104 - DNA, 24 bp, Artificial Sequence
  pBHG10 forward primer
tgtacaccggatccggcgcacacc

SEQ ID NO: 105 - DNA, 35 bp, Artificial Sequence
  pBHG10 reverse primer
cacaacgagctcaattaattaattgccacatcctc

SEQ ID NO: 106 - DNA, 32480 bp, Artificial Sequence
  Ad5._gal._F
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcatcgatgtcgacaagcttgaattcgattaatgtgagttagctcactcattaggcaccccaggctttacactttatgcttccggctcgtatgttgtgtggaattgtgagcggataacaatttcacacaggaaacagctatgaccatgattacgaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgggatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctggtaccggtgggtgaagaccagaaacagcacctcgaactgagccgcgatattgcccagcgtttcaacgcgctgtatggcgagatcgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggaacttgtttattgcagcttataatggttacaaataaagcaatagcatcacaaatttcacaaataaagcatttttttcactgcattctagttgtggtttgtccaaactcatcaatgtatcttatcatgtctggatccagatctgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatttaattaattgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 107 - protein, 12 aa, Artificial Sequence
  Linker Sequence
PSASASASAPGS

SEQ ID NO: 108 - DNA, 8383 bp, Artificial Sequence
  Plasmid pDV60
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacgagatccgtactccgccgccgagggacctgagcgagtccgcatcgaccggatcggaaaacctctcgagaaaggcgtctaaccagtcacagtcgcaagatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaaagaagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 109 - DNA, 7960 bp, Artificial Sequence
  Plasmid pDV67
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacgagatccgtactccgccgccgagggacctgagcgagtccgcatcgaccggatcggaaaacctctcgagaaaggcgtctaaccagtcacagtcgcaagatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaaagaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 110 - DNA, 30 bp, Artificial Sequence
  pGEM5TS3H forward primer
atgggatccaagatgaagcgcgcaagaccg

SEQ ID NO: 111 - DNA, 30 bp, Artificial Sequence
  pGEM5TS3H reverse primer
cactatagcggccgcattctcagtcatctt

SEQ ID NO: 112 - DNA, 7989 bp, Artificial Sequence
  Plasmid pDV69
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacgagatccgtactccgccgccgagggacctgagcgagtccgcatcgaccggatcggaaaacctctcgagaaaggcgtctaaccagtcacagtcgcaagatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgactgatttttaagaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccsnccntagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 113 - DNA, 53 bp, Artificial Sequence
  Primer
gtcactcgaggactcggtcgactgaaaatgagacatattatctgccacggacc

SEQ ID NO: 114 - DNA, 36 bp, Artificial Sequence
  Primer
cgagatcgatcacctccggtacaaggtttggcatag

SEQ ID NO: 115 - DNA, 37 bp, Artificial Sequence
  Primer
catgaagatctggaaggtgctgaggtacgatgagacc

SEQ ID NO: 116 - DNA, 51 bp, Artificial Sequence
  Primer
gcgacttaagcagtcagctgagacagcaagacacttgcttgatccaaatcc

SEQ ID NO: 117 - DNA, 38 bp, Artificial Sequence
  Primer
cacgaattcgtcagcgcttctcgtcgcgtccaagaccc

SEQ ID NO: 118 - DNA, 32 bp, Artificial Sequence
  Primer
caccccggggaggcggcggcgacggggacggg

(19) United States
(12) Patent Application Publication
(10) Pub. No.: US 2008/0124360 A1
(43) Pub. Date: May 29, 2008

(54) Adenovirus particles with enhanced infectivity of dendritic cells and particles with decreased infectivity of hepatocytes

(76) Inventor: Daniel J. Von Seggern, San Diego, CA (US)

Correspondence Address: DLA PIPER US LLP, 4365 EXECUTIVE DRIVE, SUITE 1100, SAN DIEGO, CA, 92121-2133, US

(21) Appl. No.: 10/808,758

(22) Filed: Mar. 24, 2004

Related U.S. Application Data
(60) Provisional application No. 60/459,000, filed on Mar. 28, 2003.
(60) Provisional application No. 60/467,500, filed on May 1, 2003.

(30) Foreign Application Priority Data
Jan. 24, 2003 (US) PCT/US03/02295

(51) Int. Cl. A61K 39/235; C12N 7/00; C12N 15/11; A61P 43/00; C12N 15/00; C12N 5/06; A61K 35/76

(52) U.S. Cl. 424/233.1; 435/235.1; 424/93.6; 536/23.72; 435/325; 435/320.1

(57) ABSTRACT

Provided are adenovirus vectors and the production of such vectors. In particular, adenoviruses with modified or heterologous fiber proteins for targeting to dendritic cells are provided.

### RELATED APPLICATIONS

This application claims the benefit of priority under 35 U.S.C. §119(e) to U.S. provisional application Ser. No. 60/459,000, filed Mar. 28, 2003, entitled “DETARGETING OF ADENOVIRAL PARTICLES AND USES THEREOF”, to Daniel J. Von Seggern, and to U.S. provisional application Ser. No. 60/467,500, filed May 1, 2003, entitled “PSEUDOTYPED ADENOVIRAL VECTORS WITH ENHANCED INFECTIVITY TOWARDS DENDRITIC CELLS”, to Daniel J. Von Seggern.

This application also is related to International PCT application No. (attorney docket number 22908-1239PC), filed the same day herewith, entitled “PSEUDOTYPED ADENOVIRAL VECTORS WITH ENHANCED INFECTIVITY TOWARDS DENDRITIC CELLS,” to Daniel J. Von Seggern.

This application also is related to U.S. application Ser. No. 10/403,337, filed Mar. 27, 2003 and U.S. application Ser. No. 10/351,890, filed Jan. 24, 2003, to Michael Kaleko, Glen R. Nemerow, Theodore Smith and Susan C. Stevenson, entitled “FIBER SHAFT MODIFICATIONS FOR EFFICIENT TARGETING”. This application also is related to U.S. provisional application Ser. No. 60/350,388, filed Jan. 24, 2002, entitled “FIBER SHAFT MODIFICATIONS FOR EFFICIENT TARGETING,” to Susan C. Stevenson, Michael Kaleko, Theodore Smith and Glen R. Nemerow, and to U.S. provisional application Ser. No. 60/391,967, filed Jun. 26, 2002, entitled “FIBER SHAFT MODIFICATIONS FOR EFFICIENT TARGETING,” to Stevenson, Susan C., Kaleko, Michael, Smith, Theodore and Nemerow, Glen R. This application also is related to International PCT application No. PCT/US03/02295, filed Jan. 24, 2003, entitled “FIBER SHAFT MODIFICATIONS FOR EFFICIENT TARGETING,” to Michael Kaleko, Glen R. Nemerow, Theodore Smith and Susan C. Stevenson.

The subject matter of each of these applications, provisional applications and international applications is incorporated by reference herein.

Work described and claimed herein was supported by Department of Defense Prostate Cancer Research Program DAMD17-01-1-0098 Department of Defense Breast Cancer Research Program DAMD17-01-1-0391. The government has certain rights in such subject matter.

### FIELD OF INVENTION

The present invention generally relates to the field of adenoviral vectors and the production of such vectors. Targeted and detargeted adenoviral vectors are provided. In particular, adenoviral vectors targeted to dendritic cells are provided.

### BACKGROUND

The immune system is designed to eradicate a large number of pathogens, as well as tumors, with minimal immunopathology. When the immune system becomes defective, however, numerous disease states result. Immunotherapy is an emerging treatment modality that seeks to harness the power of the human immune system to treat disease. Immunotherapy is designed either to enhance the cellular immune response in subjects with diseases characterized by immunosuppression and/or to suppress the cellular immune response in subjects with diseases characterized by an overactive cellular immune response and/or to mount an immune response against pathogens or tumors. Improved immunotherapeutic protocols are needed.

In addition, despite the extensive characterization of numerous infectious agents and the availability of vaccines, new vaccines are needed to protect against or ameliorate diseases, such as tuberculosis, malaria (Plebanski et al. (2002) J. Clin. Invest. 110:295-301), and a large number of viruses including human immunodeficiency virus (HIV), herpes simples virus (HSV), human papilloma virus (HPV), Epstein-Barr virus (EBV), hepatitis C virus (HCV), respiratory syncytial virus (RSV), parainfluenza viruses and human metapneumovirus (Letvin (2002) J. Clin. Invest. 110:15-20; Whitley and Roizman (2002) J. Clin. Invest. 110:145-151; Murphy and Collins (2002) J. Clin. Invest. 110:21-27), caused by many clinically-relevant pathogens. Although vaccines have been developed for influenza and anthrax, more effective vaccines to prevent or reduce the severity of the diseases caused by these agents are needed (see, e.g., Palese and Garcia-Sastre (2002) J. Clin. Invest. 110:9-13; Leppla et al. (2002) J. Clin. Invest. 110:141-144; Steinman and Pope (2002) J. Clin. Invest. 109:1519-1526).

Vaccines and immunotherapy have been used to eliminate or a wide variety of cancerous cells and tumors to thereby effect treatment of cancer. Many human cancers are associated with the expression of specific proteins, such as tumor antigens, thus providing a means of identifying cancerous cells from normal cells, and providing a target for immunotherapy. The immune system is capable of recognizing these tumor antigens and eliciting an immune response directed against cells displaying the tumor antigen (van der Bruggen et al. (1991) Science 254:1643-1647; Sahin et al. (1995) Proc. Natl. Acad. Sci. U.S.A. 92:11810-11813; Kaplan et al. (1998) Proc. Natl. Acad. Sci. U.S.A. 95:7556-7561). The identification of these tumor antigens has led to the development of vaccine and immunotherapeutic approaches for the treatment of cancer (Scanlan and Jäger (2001) Breast Cancer Res. 3:95-98; Yu and Restifo (2002) J. Clin. Invest. 110:28-94). There, however, remain numerous challenges in the development of effective immunotherapies. These include, for example, a need for (i) enhancing antibody and T cell-mediated immune memory, (ii) enhancing T cell responses (CD4+ T helper cells and CD8+ CTLs), (iii) establishing mucosal immunity, which is important for vaccination against many sexually transmitted diseases, (iv) development of vaccines that can diminish the immune response, which is important for the treatment of autoimmune diseases (Steinman and Pope (2002) J. Clin. Invest. 109:1519-1526) and (v) others.

Most, if not all, adenoviral vector-mediated gene therapy strategies aim to transduce a specific tissue, such as a tumor or an organ, or a specific cell type, cells as immune cells. Systemic delivery will require ablation of the normal virus tropism as well as addition of new specificities. Multiple interactions between adenoviral particles and the host cell are required to promote efficient cell entry (Nemerow (2000) Virology 274:1-4). An adenovirus entry pathway is believed to involve two separate cell surface events. First, the adenoviral fiber knob mediates attachment of the adenovirus particle to a target cell through a high affinity interaction with a specific cell-surface receptor, which is the coxsackie-adenovirus receptor (CAR) for most, but not all, serotypes of adenovirus. A subsequent association of penton with cell surface integrins αᵥβ₃ and αᵥβ₅, which act as co-receptors, potentiates virus internalization. Although there are a plurality of adenoviral fiber receptors, in addition to CAR, that interact with subgroup B (e.g., Ad3) and subgroup C (e.g., Ad5) adenoviruses, both subgroups appear to require interaction with integrins for internalization.

The role of CAR interaction for in vivo gene transfer is not clear. CAR ablation does not change biodistribution and toxicity of adenoviral vectors in vivo (Alemany et al. (2001) Gene Therapy 8:1347-1353; U.S. patent application Ser. No. 09/870,203, filed May 30, 2001, and published as U.S. Published application No. 20020137213). Published studies have described conflicting results (Alemany et al. (2001) Gene Therapy 8:1347-1353; Leissner et al. (2001) Gene Therapy 8:49-57; Einfeld et al. (2001) J. Virology 75:11284-11291). For example, it has been shown that vectors containing an S408E mutation in the Ad5 fiber AB loop yield efficient liver transduction in mice, despite having greatly reduced transduction efficiencies on cells in culture (see, Leissner et al. (2001) Gene Therapy 8:49-57). In contrast, vectors containing a more extensive fiber AB loop mutation showed a 10-fold reduction in liver gene expression (see, Einfeld et al. (2001) J. Virology 75:11284-11291).

A doubly ablated adenovirus has been prepared by modifying the CAR binding region in the fiber loop and the integrin binding region in the penton base (Einfeld et al. (2001) J. Virology 75:11284-11291). This doubly ablated adenovirus, lacking CAR and integrin interactions, was reported not only to lack in vitro transduction of various cell types but also to lack in vivo transduction of liver cells. Specifically, the doubly ablated adenovirus was reported to have a 700-fold reduction in liver transduction when compared to the non-ablated adenovirus. These results, however, were not reproduced by others.

For many applications, the most clinically useful adenoviral vector would be deliverable systemically, such as into a peripheral vein, and would be targeted to a desired location in the body or a desired cell type, and would not have undesirable side effects resulting from targeting to other locations. In vivo adenoviral vector targeting is a major goal in gene therapy and a significant effort has been focused on developing strategies to achieve this goal. Successful targeting strategies would direct the entire vector dose to the appropriate site and would be likely to improve the safety profile of the vector by permitting the use of lower, less toxic vector doses, which potentially also can be less immunogenic. Thus, there is a need to develop adenoviruses that are fully detargeted in vivo for use as a base vector for producing redirected adenoviruses.

Therefore, among the objects herein, it is an object to provide detargeted adenoviral vectors, methods for preparation thereof, and uses thereof. Furthermore, it is in an object herein to provide immunotherapeutic methods and compositions therefor.

### SUMMARY

Provided are immunotherapeutic methods and compositions that have immunotherapeutic activity. In particular, adenoviral vectors that deliver antigens to dendritic cells for processing and presentation to T cells are provided. Delivery of antigens to dendritic cells has preventive, diagnostic and therapeutic applications.

Detargeted and fully detargeted adenoviral particles from serotype C, such as adenovirus 2 and adenovirus 5, adenovirus vectors from which such particles are produced, methods for preparation of the vectors and particles and uses of the vectors and particles are provided. Retargeted particles also are provided.

The particles are detargarted from binding to certain native receptors (e.g., coxsackie-adenovirus receptor (CAR) for Ad5 and Ad2), and can be targeted to receptors expressed on dendritic cells. In addition, among the viral particles provides are particles that do not bind to or exhibit reduced binding to HSP (Heparin Sulfate Proteoglycans; also referred to as heparin sulfate glycosaminoglycans), and, hence, exhibit reduced or no binding to hepatocytes, which express HSPs.

Provided are the adenoviral particles and genomes encoding such particles and/or genomes (viral nucleic acid molecules), cell lines and methods for producing such particles. In particular genomes that encode Ad5 particles or other type C viral particles that express fibers from adenovirus subgroup D or subgroup B, such as Ad19p, Ad30, Ad37, Ad16 and Ad35 (or that express modified fibers thereof) are provided. The fibers are modified to permit incorporation into the particle. The viral particles provided herein exhibit reduced binding to hepatocytes and hence reduced liver toxicity.

Adenoviral particles that contain a heterologous fiber or a portion thereof, whereby binding of the viral particle to dendritic cells is increased and binding to heparin sulfate proteoglycans (HSP) and to CAR is reduced or eliminated compared to a particle that expresses its native fiber are provided. In these particles, the adenoviral (Ad) particle, except for the fiber, is from a subgroup C adenovirus; and the fiber is from an adenovirus subgroup D, such as Ad19p. In another embodiment, the heterologous fiber is from Ad30. In other embodiments, the fiber is chimeric and comprises an N-terminal portion from a fiber of a subgroup C Ad virus; and the N-terminal portion is sufficient to increase incorporation into the particle compared to in its absence. For example, the fiber can be from an adenovirus Ad19p, Ad30, Ad37, Ad16 or Ad35 virus. The fiber protein can additionally include one or more further modifications that reduce or eliminate interaction of the resulting fiber with one or more cell surface proteins, such as CAR, in addition to HSP.

The adenoviral particle also can include a mutation in the CAR-binding region of the capsid and/or a mutation in the αᵥ integrin-binding region of the capsid, whereby binding to the integrin is eliminated or reduced. The CAR-binding region of the capsid that is modified can be on a fiber knob.

In some embodiments, the chimeric fiber contains at least a sufficient number of amino acids of Ad19p fiber set forth as SEQ ID No. 34 to target a particle to dendritic cells, and optionally to reduce or eliminate binding of the particle to HSP. For example, the Ad19p fiber is modified by replacing at least the N-terminal 15, 16 or 17 amino acids with the 15, 16 or 17 amino acids of an Ad2 or Ad5 fiber. In other embodiments, the chimeric fiber contains at least a sufficient number of amino acids of Ad30 fiber set forth as SEQ ID No. 36 to target a particle to dendritic cells, and optionally to reduce or eliminate binding of the particle to HSP. For example, the Ad30 fiber is modified by replacing at least the N-terminal 15, 16 or 17 amino acids with the 15, 16 or 17 amino acids of an Ad2 or Ad5 fiber.

Hence, also provided are methods for making and using the adenoviral particles that express the modified fibers and combinations of modified fibers and modified penton. With the fiber shaft modifications, particularly in combination with the fiber knob modifications and the penton modifications, the adenovirus particles are ablated for binding to their natural cellular receptor(s), i.e., they are detargeted. In addition, by selection of a subgroup D fiber, the resulting particles are targeted to dendritic cells. The particles also can be “retargeted” to a specific cell type through the addition of a ligand to the virus capsid, which causes the virus to bind to and infect such cell. The ligand can be added, for example, through genetic modification of a capsid protein gene.

The nucleic acids, proteins, adenoviral particles and adenoviral vectors have a variety of uses. These include in vivo and in vitro uses to target nucleic acid to particular cells and tissues, for therapeutic purposes, including gene therapy, and also for the identification and study of cell surface receptors and identification of modes of interaction of viruses with cells.

Nucleic acids encoding the capsid proteins, including the fibers are also provided. The nucleic acids can be provided as vectors, particularly as adenovirus vectors. Many adenoviral vectors are known and can be modified as needed in accord with the description herein. Adenoviral vectors include, but are not limited to, early generation adenoviral vectors, such as E1-deleted vectors, gutless adenoviral vectors and replication-conditional adenoviral vectors, such as oncolytic adenoviral vectors. The adenovirus vectors also can include heterologous nucleic acids that encode or provide products, such as tumor antigens and antigens from pathogens that induce an immunotherapeutic response whereby infection with such pathogen is prevented or the symptoms of infection reduced. Heterologous nucleic acid can encode a polypeptide or comprise or encode a regulatory sequence, such as a promoter or an RNA, including RNAi, small RNAs, other double-stranded RNAs, antisense RNA, and ribozymes. Promoters include, for example, constitutive and regulated promoters and tissue specific promoters, including tumor specific promoters. The promoter can be operably linked, for example, to a gene of an adenovirus essential for replication.

Cells containing the nucleic acid molecules and cells containing the vectors are also provided. Such cells include packaging cells. The cells can be prokaryotic or eukaryotic cells, including mammalian cells, such as primate cells, including human cells.

Also provided are adenoviral particles that contain the modified capsid proteins provided herein. The particles have increased tropism for dendritic cells, and also exhibit altered interaction or binding with HSP compared to particles that do not contain the modified capsid proteins. In addition to altered binding to HSP and dendritic cells, the particles can include further modifications, such as capsid proteins with altered interaction with other receptors as described above. In particular, the particles can have altered, typically reduced or eliminated, interaction with CAR, αᵥ integrin and/or other receptors. The mutations include mutations in the fiber knob, penton and hexon. Exemplary fiber knob mutations are mutations in the AB loop or CD loop, such as KO1 or KO12. Such mutations include, for example, PD1, KO1, KO12 and S* (see, e.g., U.S. provisional application Ser. No. 60/459,000, and copending U.S. application Ser. No. 10/351,890). In addition, the particles can include additional ligands for retargeting to selected receptors. The adenoviral particles can be from any serotype and subgroup.

Methods for expressing heterologous nucleic acids in a cell are provided. In these methods an adenoviral vector provided herein is transduced into a cell to deliver the nucleic acid and/or encoded products. Transduction can be effected in vivo or in vitro or ex vivo, and can be for a variety of purposes including study of gene expression and genetic therapy. The cells can be prokaryotic cells, but typically are eukaryotic cells, including mammalian cells, such as primate, including human cells. The cells can be of a specific type, such as a tumor cell or a cell in a particular tissue.

### BRIEF DESCRIPTION OF THE FIGURES

FIG. 1 is a plasmid map for pSKO1.

FIG. 2 is a plasmid map for pNDSQ3.1KO1.

FIGS. 3A-3C are plasmid maps of pAdmireRSVnBg (FIG. 3A), pSQ1 (FIG. 3B) and pSQ1KO12 (FIG. 3C).

FIG. 4 is a plasmid map for pSQ1 PD1.

FIGS. 5A-5B are plasmid maps of pSQ1FKO1PD1 (FIG. 5A) and pSQ1KO12PD1 (FIG. 5B).

FIG. 6 shows in vitro transduction efficiency of A549 cells using adenoviral vectors containing fiber AB loop knob and/or penton, PD1 mutations. The following adenoviral vectors were used in these studies: Av1nBg, Av1nBgFKO1, referred to as FKO1, Av1nBgPD1, referred to as PD1, and Av1nBgFK01PD1 that is referred to as FK01PD1.

FIG. 7A-7B shows in vivo adenoviral-mediated liver gene expression (FIG. 7A) and hexon DNA content (FIG. 7B) using adenoviral vectors containing fiber AB loop knob and/or penton, PD1 mutations. The following adenoviral vectors were used in these studies: Av1nBg, Av1nBgFKO1, referred to as FKO1, Av1nBgPD1, referred to as PD1, Av1nBgFKO1PD1, referred to as FKO1PD1, Av1nBgKO12, referred to as KO12, and Av1nBgKO12PD1 that is referred to as KO12PD1.

FIG. 8 is a plasmid map for pFBshuttle(EcoRI).

FIG. 9 is a plasmid map for pSQ1 HSP.

FIG. 10 is a plasmid map for pSQ1HSPKO1.

FIG. 11 is a plasmid map for pSQ1HSPPD1.

FIG. 12 is a plasmid map for pSQ1HSPKO1PD1.

FIGS. 13A-13C show the transduction efficiency of A549 and HeLa cells using adenoviral vectors containing fiber shaft, knob and/or penton mutations. FIG. 13A shows the dose response for the transduction efficiency of A549 cells. FIG. 13B shows the transduction efficiency of HeLa cells at 2000 ppc. FIG. 13C shows the competition analysis of adenoviral vectors containing fiber shaft mutations.

FIGS. 14A-14B shows the influence of fiber shaft mutations on in vivo adenoviral-mediated liver gene expression (FIG. 14A) and hexon DNA content (FIG. 14B).

FIGS. 15A-15B are plasmid maps of pSQ1 HSPRGD (FIG. 15A) and pSQ1HSPKO1RGD (FIG. 15B).

FIG. 16 shows that insertion of a RGD targeting ligand can restore transduction of the vectors containing the HSP binding shaft S* mutation.

FIGS. 17A-17B are plasmid maps of pSQ1AD35Fiber (FIG. 17A) and pSQ1Ad35FcRGD (FIG. 17B).

FIGS. 18A-18B are maps of plasmids encoding 35F chimeric fibers. FIG. 18A is a plasmid map of pSQ135T5H, and FIG. 18B is a plasmid map of pSQ15T35H.

FIG. 19 shows the results of an in vitro analysis of Ad5 vectors containing Ad35 fibers and derivatives thereof.

FIG. 20 shows the results of an in vivo analysis of Ad5 vectors containing Ad35 fibers and derivatives thereof.

FIGS. 21A-21B are plasmid maps of pSQ1Ad41sF (FIG. 21A) and pSQ1Ad41sFRGD (FIG. 21B).

FIG. 22 shows the results of an in vivo analysis of Ad5 vectors containing Ad41 short fiber.

FIG. 23 shows the in vitro analysis of Ad5 based vectors containing the Ad41 short fiber which has been re-engineered to contain a cRGD ligand in the HI loop.

FIG. 24 shows enhanced transduction of AE1-2a cells with the Av3nBgFKO1 detargeted adenoviral vector using hexadimethrine bromide (HB), protamine sulfate (PS) and poly-lysine-RGD (K14) or the anti-penton-TNFα bifunctional protein (αpen-TNF).

FIG. 25 shows ablation of HSP interaction decreases adenoviral-mediated gene transfer to other organs.

FIG. 26 shows in vivo liver transduction with adenoviral vectors which encode for β-galactosidase and contain various mutations to the fiber and/or penton proteins. Results are plotted as percent transduction as compared to wild type. Two different methods for determining the level of transduction are shown for each vector.

FIG. 27 shows the adenoviral vector biodistribution to the liver and tumor for the vectors containing the S*, KO1S*, and 41sF fibers.

### DETAILED DESCRIPTION

A. DEFINITIONS
B. Adenovirus-cell interactions
   1. Fiber protein
   2. Pseudotyping
C. Dendritic cell targeting
   1. Dendritic cells
   2. Dendritic cell therapies
   3. Targeting adenoviral particles to dendritic cells
      a. Fiber substitution
      b. Efficient targeting
   4. Additional modifications
D. Adenovirus vector detargating
E. Nucleic acids, adenoviral vectors and cells containing the nucleic acids and cells containing the vectors
   1. Preparation of viral particles
   2. Adenoviral vectors and particles
      a. Gutless vectors
      b. Oncolytic vectors
   3. Packaging
   4. Propagation and scale-up
F. Adenovirus expression vector systems
   1. Nucleic acid gene expression cassettes
   2. Promoters
G. Heterologous polynucleotides and therapeutic nucleic acids
H. Formulation and administration
   1. Formulation
   2. Administration
I. Diseases, disorders and therapeutic products
J. Examples

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information is known and can be readily accessed, such as by searching the internet and/or appropriate databases. Reference thereto evidences the availability and public dissemination of such information.

As used herein, the term “adenovirus” or “adenoviral particle” is used to include any and all viruses that can be categorized as an adenovirus, including any adenovirus that infects a human or an animal, including all groups, subgroups, and serotypes. Depending upon the context reference to “adenovirus” can include adenoviral vectors. There are at least 51 serotypes of adenovirus that are classified into several subgroups. For example, subgroup A includes adenovirus serotypes 12, 18, and 31. Subgroup C includes adenovirus serotypes 1, 2, 5, and 6. Subgroup D includes adenovirus serotypes 8, 9, 10, 13, 15, 17, 19, 19p, 20, 22-30, 32, 33, 36-39, and 42-49. Subgroup E includes adenovirus serotype 4. Subgroup F includes adenovirus serotypes 40 and 41. These latter two serotypes have a long and a short fiber protein. Thus, as used herein an adenovirus or adenovirus particle is a packaged vector or genome.

As used herein, “virus,” “viral particle,” “vector particle,” “viral vector particle,” and “virion” are used interchangeably to refer to infectious viral particles that are formed when, such as when a vector containing all or a part of a viral genome, is transduced into an appropriate cell or cell line for the generation of such particles. The resulting viral particles have a variety of uses, including, but not limited to, transferring nucleic acids into cells either in vitro or in vivo. For purposes herein, the viruses are adenoviruses, including recombinant adenoviruses formed when an adenovirus vector, such as any provided herein, is encapsulated in an adenovirus capsid. Thus, a viral particle is a packaged viral genome. An adenovirus viral particle is the minimal structural or functional unit of a virus. A virus can refer to a single particle, a stock of particles or a viral genome. The adenovirus (Ad) particle is relatively complex and may be resolved into various substructures.

Included among adenoviruses and adenoviral particles are any and all viruses that can be categorized as an adenovirus, including any adenovirus that infects a human or an animal, including all groups, subgroups, and serotypes. Thus, as used herein, “adenovirus” and “adenovirus particle” refer to the virus itself and derivatives thereof and cover all serotypes and subtypes and naturally occurring and recombinant forms, except where indicated otherwise. Included are adenoviruses that infect human cells. Adenoviruses can be wildtype or can be modified in various ways known in the art or as disclosed herein. Such modifications include, but are not limited to, modifications to the adenovirus genome that is packaged in the particle in order to make an infectious virus. Exemplary modifications include deletions known in the art, such as deletions in one or more of the E1a, E1b, E2a, E2b, E3, or E4 coding regions. Other exemplary modifications include deletions of all of the coding regions of the adenoviral genome. Such adenoviruses are known as “gutless” adenoviruses. The terms also include replication-conditional adenoviruses, which are viruses that preferentially replicate in certain types of cells or tissues but to a lesser degree or not at all in other types. For example, among the adenoviral particles provided herein, are adenoviral particles that replicate in abnormally proliferating tissue, such as solid tumors and other neoplasms. These include the viruses disclosed in U.S. Pat. No. 5,998,205 and U.S. Pat. No. 5,801,029. Such viruses are sometimes referred to as “cytolytic” or “cytopathic” viruses (or vectors), and if they have such an effect on neoplastic cells, are referred to as “oncolytic” viruses (or vectors).

As used herein, the terms “vector,” “polynucleotide vector,” “polynucleotide vector construct,” “nucleic acid vector construct,” and “vector construct” are used interchangeably herein to mean any nucleic acid construct that can be used for gene transfer, as understood by those skilled in the art.

As used herein, the term “viral vector” is used according to its art-recognized meaning. It refers to a nucleic acid vector construct that includes at least one element of viral origin and can be packaged into a viral vector particle. The viral vector particles can be used for the purpose of transferring DNA, RNA or other nucleic acids into cells either in vitro or in vivo. Viral vectors include, but are not limited to, retroviral vectors, vaccinia vectors, lentiviral vectors, herpes virus vectors (e.g., HSV), baculoviral vectors, cytomegalovirus (CMV) vectors, papillomavirus vectors, simian virus (SV40) vectors, Sindbis vectors, Semliki Forest virus vectors, phage vectors, adenoviral vectors, and adeno-associated viral (AAV) vectors. Suitable viral vectors are described, for example, in U.S. Pat. Nos. 6,057,155, 5,543,328 and 5,756,086. The vectors provided herein are adenoviral vectors.

As used herein, “adenovirus vector” and “adenoviral vector” are used interchangeably and are well understood in the art to mean a polynucleotide containing all or a portion of an adenovirus genome. An adenoviral vector, refers to nucleic encoding a complete genome or a modified genome or one that can be used to introduce heterologous nucleic acid when transferred into a cell, particularly when packaged as a particle. An adenoviral vector can be in any of several forms, including, but not limited to, naked DNA, DNA encapsulated in an adenovirus capsid, DNA packaged in another viral or viral-like form (such as herpes simplex, and AAV), DNA encapsulated in liposomes, DNA complexed with polylysine, complexed with synthetic polycationic molecules, conjugated with transferrin, complexed with compounds such as PEG to immunologically “mask” the molecule and/or increase half-life, or conjugated to a non-viral protein.

As used herein, oncolytic adenoviruses refer to adenoviruses that replicate selectively in tumor cells

As used herein, a variety of vectors with different requirements and purposes are described. For example, one vector is used to deliver particular nucleic acid molecules into a packaging cell line for stable integration into a chromosome. These types of vectors also are referred to as complementing plasmids. A further type of vector carries or delivers nucleic acid molecules in or into a cell line (e.g., a packaging cell line) for the purpose of propagating viral vectors; hence, these vectors also can be referred to herein as delivery plasmids. A third “type” of vector is the vector that is in the form of a virus particle encapsulating a viral nucleic acid and that is comprised of the capsid modified as provided herein. Such vectors also can contain heterologous nucleic acid molecules encoding particular polypeptides, such as therapeutic polypeptides or regulatory proteins or regulatory sequences to specific cells or cell types in a subject in need of treatment.

As used herein, the term “motif” is used to refer to any set of amino acids forming part of a primary sequence of a protein, either contiguous or capable of being aligned to certain positions that are invariant or conserved, that is associated with a particular function. The motif can occur not only by virtue of the primary sequence, but also as a consequence of three-dimensional folding. For example, the adenovirus fiber is a trimer, hence the trimeric structure can contribute to formation of a motif. Alternatively, a motif can be considered as a domain of a protein, where domain is a region of a protein molecule delimited on the basis of function without knowledge of and relation to the molecular substructure, as, e.g., the part of a protein molecule that binds to a receptor. As shown herein, the motif KKTK constitutes a consensus sequence for fiber shaft interaction with HSP.

As used herein, cell therapy is a method of treatment involving the administration of live cells. Adoptive immunotherapy is a treatment process involving removal of cells from a subject, the processing of the cells in some manner ex-vivo and the infusion of the processed cells into the same or different subject as a therapy.

As used herein, a cell therapeutic refers to the compositions of cells that are formulated as a drug whose active ingredient is wholly or in part a living cell.

As used herein, immune cells are the subset of blood cells known as white blood cells, which include mononuclear cells such as lymphocytes, monocytes, macrophages and granulocytes.

As used herein, T-cells are lymphocytes that express the CD3 antigen.

As used herein, helper cells are CD4+ lymphocytes.

As used herein, regulatory cells are a subset of T-cells, most commonly CD4+ T-cells, that are capable of enhancing or suppressing an immune response. Regulatory immune cells regulate an immune response primarily by virtue of their cytokine secretion profile. Some regulatory immune cells also can act to enhance or suppress an immune response by virtue of antigens expressed on their cell surface and mediate their effects through cell-to-cell contact. Th1 and Th2 cells are examples of regulatory cells.

As used herein, effector cells are immune cells that primarily act to eliminate tumors or pathogens through direct interaction, such as phagocytosis, perforin and/or granulozyme secretion, induction of apoptosis, etc. Effector cells generally require the support of regulatory cells to function and also act as the mediators of delayed type hypersensitivity reactions and cytotoxic functions. Examples of effector cells are B lymphocytes, macrophages, cytotoxic lymphocytes, LAK cells, NK cells and neutrophils.

As used herein, a professional antigen presenting cells (APC) include dendritic cells, B-cells and macrophages.

As used herein, the term “bind” or “binding” is used to refer to the binding between a ligand and its receptor, such as the binding of the Ad5 knob domain with CAR (coxsackie-adenovirus receptor), with a K_{d} in the range of 10-2 to 10-15 mole/l, generally, 10⁻⁶ to 10⁻¹⁵, 10⁻⁷ to 10⁻¹⁵ and typically 10⁻⁸ to 10⁻¹⁵ (and/or a Kₐ of 10⁵-10¹², 10⁷-10¹², 10¹⁸-10¹² l/mole).

As used herein, specific binding or selective binding means that the binding of a particular ligand and one receptor interaction (kₐ or K_{eq}) is at least 2-fold, generally, 5, 10, 50, 100 or more-fold, greater than for another receptor. A statement that a particular viral vector is targeted to a cell or tissue means that its affinity for such cell or tissue in a host or in vitro is at least about 2-fold, generally, 5, 10, 50, 100 or more-fold, greater than for other cells and tissues in the host or under the in vitro conditions.

As used herein, the term “ablate” or “ablated” is used to refer to an adenovirus, adenoviral vector or adenoviral particle, in which the ability to bind to a particular cellular receptor is reduced or eliminated, generally substantially eliminated (i.e., reduced more than 10-fold, 100-fold or more) when compared to a corresponding wild-type adenovirus. An ablated adenovirus, adenoviral vector or adenoviral particle also is said to be detargeted, i.e., the modified adenovirus, adenoviral vector or adenoviral particle does not possess the native tropism of the wild-type adenovirus. The reduction or elimination of the ability of the mutated adenovirus fiber protein to bind a cellular receptor as compared to the corresponding wild-type fiber protein can be measured or assessed by comparing the transduction efficiency (gene transfer and expression of a marker gene) of an adenovirus particle containing the mutated fiber protein compared to an adenovirus particle containing the wild-type fiber protein for cells having the cellular receptor.

As used herein, tropism with reference to an adenovirus refers to the selective infectivity or binding that is conferred on the particle by a capsid protein, such as the fiber protein and/or penton.

As used herein, “penton” or “penton complex” is used herein to designate a complex of penton base and fiber. The term “penton” can also be used to indicate penton base, as well as penton complex. The meaning of the term “penton” alone should be clear from the context within which it is used.

As used herein, the term “substantially eliminated” refers to a transduction efficiency less than about 11% of the efficiency of the wild-type fiber containing virus on HeLa cells. The transduction efficiency on Hela cells can be measured (see, e.g., Example 1 of U.S. patent application Ser. No. 09/870,203 filed on May 30, 2001, and published as U.S. Published application No. 20020137213, and of International Patent Application No. PCT/EP01/06286 filed Jun. 1, 2001, and published as WO 01/92299). Briefly, HeLa cells are infected with the adenoviral vectors containing mutated fiber proteins to evaluate the effects of fiber amino acid mutations on CAR interaction and subsequent gene expression. Monolayers of HeLa cells in 12 well dishes are infected with, for example, 1000 particles per cell for 2 hours at 37° C. in a total volume of, for example, 0.35 ml of the DMEM containing 2% FBS. The infection medium is then aspirated from the monolayers and 1 ml of complete DMEM containing 10% FBS was added per well. The cells are incubated for an sufficient time, generally about 24 hours, to allow for 8-galactosidase expression, which is measured by a chemiluminescence reporter assay and by histochemical staining with a chromogenic substrate. The relative levels of β-galactosidase activity are determined using as suitable system, such as the Galacto-Light chemiluminescence reporter assay system (Tropix, Bedford, Mass.). Cell monolayers are washed with PBS and processed according to the manufacturer's protocol. The cell homogenate is transferred to a microfuge tube and centrifuged to remove cellular debris. Total protein concentration is determined, such as by using the bicinchoninic acid (BCA) protein assay (Pierce, Inc., Rockford, Ill.) with bovine serum albumin as the assay standard. An aliquot of each sample is then incubated with the Tropix β-galactosidase substrate for 45 minutes in a 96 well plate. A luminometer is used determine the relative light units (RLU) emitted per sample and then normalized for the amount of total protein in each sample (RLU/ug total protein). For the histochemical staining procedure, the cell monolayers are fixed with 0.5% glutaraldehyde in PBS, and then were incubated with a mixture of 1 mg of 5-bromo-4-chloro-3-indolyl-β-D-galactoside (X-gal) per ml, 5 mM potassium ferrocyanide, 5 mM potassium ferricyanide and 2 mM MgCl₂ in 0.5 ml of PBS. The monolayers are washed with PBS and the blue cells are visualized by light microscopy, such as with a Zeiss IDO3 microscope. Generally, the efficiency is less than about 9%, and typically is less than about 8%.

As used herein, the phrase “reduce” or “reduction” refers to a change in the efficiency of transduction by the adenovirus containing the mutated or heterologous fiber as compared to the adenovirus containing the wild-type fiber to a level of about 75% or less of the wild-type on HeLa cells. Generally, the change in efficiency is to a level of about 65% or less than wild-type. Typically it is about 55% or less. This system is able to rapidly analyze modified fiber proteins and/or modified penton proteins for desired tropism in the context of the viral particle.

As used herein, the term “mutate” or “mutation” or similar terms refers to the deletion, insertion or change of at least one amino acid in the protein of interest (e.g. the part of the fiber shaft region interacting with HSP). The amino acid can be changed by substitution or by modification in a way that derivatizes the amino acid.

As used herein, the term “polynucleotide” means a nucleic acid molecule, such as DNA or RNA, that encodes a polynucleotide. The molecule can include regulatory sequences, and is generally DNA. Such polynucleotides are prepared or obtained by techniques known by those skilled in the art in combination with the teachings contained therein.

As used herein, the term “viral vector” is used according to its art-recognized meaning. It refers to a nucleic acid vector construct that includes at least one element of viral origin and can be packaged into a viral vector particle. The viral vector particles can be used, for example, for transferring DNA into cells either in vitro or in vivo.

As used herein, adenoviral genome is intended to include any adenoviral vector or any nucleic acid sequence comprising a modified fiber protein. All adenovirus serotypes are contemplated for use in the vectors and methods herein.

As used herein, a packaging cell line is a cell line that is able to package adenoviral genomes or modified genomes to produce viral particles. It can provide a missing gene product or its equivalent. Thus, packaging cells can provide complementing functions for the genes deleted in an adenoviral genome (e.g., the nucleic acids encoding modified fiber proteins) and are able to package the adenoviral genomes into the adenovirus particle. The production of such particles require that the genome be replicated and that those proteins necessary for assembling an infectious virus are produced. The particles also can require certain proteins necessary for the maturation of the viral particle. Such proteins can be provided by the vector or by the packaging cell.

As used herein, detargeted adenoviral particles have ablated (reduced or eliminated) interaction with receptors with which native particles. It is understood that in vivo no particles are fully ablated such that they do not interact with any cells. Detargeted particles have reduced, typically substantially reduced, or eliminated interaction with native receptors. For purposes herein, detargeted particles have reduced (2-fold, 5-fold, 10-fold, 100-fold or more) binding or virtually no binding to CAR or another native receptor. The particles still bind to cells, but the types of cells and interactions are reduced.

As used herein, pseudotyping describes the production of adenoviral vectors having modified capsid protein or capsid proteins from a different serotype from the serotype of the vector itself. One example, is the production of an adenovirus 5 vector particle containing an Ad37 or Ad35 fiber protein. This can be accomplished by producing the adenoviral vector in packaging cell lines expressing different fiber proteins.

As used herein, receptor refers to a biologically active molecule that specifically or selectively binds to (or with) other molecules. The term “receptor protein” can be used to more specifically indicate the proteinaceous nature of a specific receptor.

As used herein, the term “heterologous polynucleotide” means a polynucleotide derived from a biological source other than an adenovirus or from an adenovirus of a different strain or can be a polynucleotide that is in a different locus from wild-type virus. The heterologous polynucleotide can encode a polypeptide, such as a toxin or a therapeutic protein. The heterologous polynucleotide can contain regulatory regions, such as a promoter regions, such as a promoter active in specific cells or tissue, for example, tumor tissue as found in oncolytic adenoviruses. Alternatively, the heterologous polynucleotide can encode a polypeptide and further contain a promoter region operably linked to the coding region.

As used herein, the term “cyclic RGD” (or cRGD) refers to any amino acid that binds to αᵥ integrins on the surface of cells and contains the sequence RGD (Arg-Gly-Asp).

As used herein, the KO mutations refer to mutations in fiber that knock out binding to CAR. For example, a KO1 mutation refers to a mutation in the Ad5 fiber and corresponding mutations in other fiber proteins. In Ad5, this mutation results in a substitution of fiber amino acids 408 and 409, changing them from serine and proline to glutamic acid and alanine, respectively. As used herein, a KO12 mutation refers to a mutation in the Ad5 fiber and corresponding mutations in other fiber proteins. In Ad5, this mutation is a four amino acid substitution as follows: R512S, A515G, E516G, and K517G. Other KO mutations can be identified empirically or are known to those of skill in the art.

As used herein, PD mutations refer to mutations in the penton gene that ablate binding by the encoded to aV integrin by replacing the RGD tripeptide. The PD1 mutation exemplified herein results in a substitution of amino acids 337 through 344 of the Ad5 penton protein, HAIRGDTF (SEQ ID NO. 49), with amino acids SRGYPYDVPDYAGTS (SEQ ID NO. 50), thereby replacing the RGD tripeptide.

As used herein, reference to an amino acid in an adenovirus protein or to a nucleotide in an adenovirus genome is with reference to Ad5, unless specified otherwise. Corresponding amino acids and nucleotides in other adenovirus strains and modified strains and in vectors can be identified by those of skill in the art. Thus, recitation of a mutation is intended to encompass all adenovirus strains that possess a corresponding locus.

As used herein, tumor antigen refers to a cell surface protein expressed or located on the surface of tumor cells.

As used herein, treatment means any manner in which the symptoms of a condition, disorder or disease are ameliorated or otherwise beneficially altered.

As used herein, a therapeutically effective product is a product that is encoded by heterologous DNA that, upon introduction of the DNA into a host, a product is expressed that effectively ameliorates or eliminates the symptoms, manifestations of an inherited or acquired disease or that cures said disease.

As used herein, a subject is an animal, such as a mammal, typically a human, including patients.

As used herein, genetic therapy involves the transfer of heterologous DNA to the certain cells, target cells, of a mammal, particularly a human, with a disorder or conditions for which such therapy is sought. The DNA is introduced into the selected target cells in a manner such that the heterologous DNA is expressed and a therapeutic product encoded thereby is produced. Alternatively, the heterologous DNA may in some manner mediate expression of DNA that encodes the therapeutic product, it may encode a product, such as a peptide or RNA that in some manner mediates, directly or indirectly, expression of a therapeutic product. Genetic therapy may also be used to deliver nucleic acid encoding a gene product to replace a defective gene or supplement a gene product produced by the mammal or the cell in which it is introduced. The introduced nucleic acid may encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous DNA encoding the therapeutic product may be modified prior to introduction into the cells of the afflicted host in order to enhance or otherwise alter the product or expression thereof.

As used herein, a therapeutic nucleic acid is a nucleic acid that encodes a therapeutic product. The product can be nucleic acid, such as a regulatory sequence or gene, or can encode a protein that has a therapeutic activity or effect. For example, therapeutic nucleic acid can be a ribozyme, antisense, double-stranded RNA, a nucleic acid encoding a protein and others.

As used herein, “homologous” means about greater than 25% nucleic acid sequence identity, such as 25%, 40%, 60%, 70%, 80%, 90% or 95%. If necessary the percentage homology will be specified. The terms “homology” and “identity” are often used interchangeably. In general, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence identity, the number of conserved amino acids are determined by standard alignment algorithms programs, and are used with default gap penalties established by each supplier. Substantially homologous nucleic acid molecules would hybridize typically at moderate stringency or at high stringency all along the length of the nucleic acid or along at least about 70%, 80% or 90% of the full-length nucleic acid molecule of interest. Also contemplated are nucleic acid molecules that contain degenerate codons in place of codons in the hybridizing nucleic acid molecule.

Whether any two nucleic acid molecules have nucleotide sequences that are at least, for example, 80%, 85%, 90%, 95%, 96%, 97%, 98% or 99% “identical” can be determined using known computer algorithms such as the “FAST A” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA (Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps. Therefore, as used herein, the term “identity” represents a comparison between a test and a reference polypeptide or polynucleotide.

As used herein, the term “at least 90% identical to” refers to percent identities from 90 to 99.99 relative to the reference polypeptides. Identity at a level of 90% or more is indicative of the fact that, assuming for exemplification purposes a test and reference polynucleotide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g. 10/100 amino acid difference (approximately 90% identity). Differences are defined as nucleic acid or amino acid substitutions, or deletions. At the level of homologies or identities above about 85-90%, the result should be independent of the program and gap parameters set; such high levels of identity can be assessed readily, often without relying on software.

As used herein: stringency of hybridization in determining percentage mismatch is as follows:

1) high stringency: 0.1×SSPE, 0.1% SDS, 65° C.

2) medium stringency: 0.2×SSPE, 0.1% SDS, 50° C.

3) low stringency: 1.0×SSPE, 0.1% SDS, 50° C.

Those of skill in this art know that the washing step selects for stable hybrids and also know the ingredients of SSPE (see, e.g., Sambrook, E. F. Fritsch, T. Maniatis, in: Molecular Cloning, A Laboratory Manual, Cold Spring Harbor Laboratory Press (1989), vol. 3, p. B.13, see, also, numerous catalogs that describe commonly used laboratory solutions). SSPE is pH 7.4 phosphate-buffered 0.18 M NaCl. Further, those of skill in the art recognize that the stability of hybrids is determined by Tₘ, which is a function of the sodium ion concentration and temperature (Tₘ=81.5° C.-16.6(log₁₀[Na⁺])+0.41 (% G+C)−600/I)), so that the only parameters in the wash conditions critical to hybrid stability are sodium ion concentration in the SSPE (or SSC) and temperature.

It is understood that equivalent stringencies can be achieved using alternative buffers, salts and temperatures. By way of example and not limitation, procedures using conditions of low stringency are as follows (see also Shilo and Weinberg, Proc. Natl. Acad. Sci. USA 78:6789-6792 (1981)): Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA (10×SSC is 1.5 M sodium chloride, and 0.15 M sodium citrate, adjusted to a pH of 7).

Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 hours at 40° C., and then washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours at 60° C. Filters are blotted dry and exposed for autoradiography. If necessary, filters are washed for a third time at 65-68° C. and reexposed to film. Other conditions of low stringency which can be used are well known in the art (e.g., as employed for cross-species hybridizations).

By way of example and not way of limitation, procedures using conditions of moderate stringency include, for example, but are not limited to, procedures using such conditions of moderate stringency are as follows: Filters containing DNA are pretreated for 6 hours at 55° C. in a solution containing 6×SSC, 5× Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 18-20 hours at 55° C., and then washed twice for 30 minutes at 60° C. in a solution containing 1×SSC and 0.1% SDS. Filters are blotted dry and exposed for autoradiography. Other conditions of moderate stringency which can be used are well-known in the art. Washing of filters is done at 37° C. for 1 hour in a solution containing 2×SSC, 0.1% SDS.

By way of example and not way of limitation, procedures using conditions of high stringency are as follows: Prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 minutes before autoradiography. Other conditions of high stringency which can be used are well known in the art.

The term substantially identical or substantially homologous or similar varies with the context as understood by those skilled in the relevant art and generally means at least 60% or 70%, preferably means at least 80%, 85% or more preferably at least 90%, and most preferably at least 95% identity.

As used herein, substantially identical to a product means sufficiently similar so that the property of interest is sufficiently unchanged so that the substantially identical product can be used in place of the product.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound can, however, be a mixture of stereoisomers or isomers. In such instances, further purification might increase the specific activity of the compound.

The methods and preparation of products provided herein, unless otherwise indicated, employ conventional techniques of chemistry, molecular biology, microbiology, recombinant DNA, genetics, immunology, cell biology, cell culture and transgenic biology, which are within the skill of the art (see, e.g., Maniatis et al. (1982) Molecular Cloning: A Laboratory Manual, Cold Spring Harbor Laboratory Press, Cold Spring Harbor, N.Y.); Sambrook et al. (1989) Molecular Cloning: A Laboratory Manual, Cold Spring Harbor Laboratory Press, Cold Spring Harbor, N.Y.; Ausubel et al. (1992) Current Protocols in Molecular Biology, Wiley and Sons, New York; Glover (1985) DNA Cloning I and II, Oxford Press; Anand (1992) Techniques for the Analysis of Complex Genomes (Academic Press); Guthrie and Fink (1991) Guide to Yeast Genetics and Molecular Biology, Academic Press; Harlow and Lane (1988) Antibodies: A Laboratory Manual, Cold Spring Harbor Laboratory, Cold Spring Harbor, N.Y.; Jakoby and Pastan, eds. (1979) Cell Culture. Methods in Enzymology 58, Academic Press, Inc., Harcourt Brace Jaovanovich, NY; Nucleic Acid Hybridization (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal (1984), A Practical Guide To Molecular Cloning; Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Hogan et al. (1986) Manipulating the Mouse Embryo, Cold Spring Harbor Laboratory Press, Cold Spring Harbor, N.Y.

### B. ADENOVIRUS-CELL INTERACTIONS

The ability of different subgroups of adenovirus to interact with, or not interact with, specific cell types and/or particular receptors can be exploited to produce adenoviruses with desired specificity. For example, adenovirus can be modified such that they are able to more efficiently target specific cell types and/or tissues. Adenovirus serotypes also can be modified to reduce or eliminate their interactions with a natural receptor and thereby reduce or eliminate the interaction of adenovirus with a particular cell type and/or tissue. Thus, provided herein are modifications of the viral capsid that alter the interaction of an adenovirus with its natural receptors and/or cell types and modifications that target an adenovirus to interact with other receptors and/or cell types. In particular, modifications that result targeting to dendritic cells are provided. Also provided are modifications that result in reduction or ablation of the interaction of an adenovirus, particularly in vivo, with other cell types.

Different adenovirus serotypes infect different cell types, largely because their fibers bind distinct receptors (Defer et al. (1990) J. Virol. 64:3661-3673; Stevenson et al. (1995) J. Virol. 69:2850-2857; Arnberg et al. (2000) J. Virol. 74:42-48). For subgroup C viruses (including Ad2 and Ad5), coxsackievirus and adenovirus receptor (CAR) serves as the cellular receptor (Tomko and Philipson (1997) Proc. Natl. Acad. Sci. U.S.A. 94:3352-3356; Bergelson et al. (1997) Science 275:1320-1323). While adenoviruses from several other subgroups also bind CAR (Roelvink et al. (1998) J. Virol. 72:7909-7915), infection and competition studies indicate that they use other proteins as primary receptors (Arnberg et al. (2000) J. Virol. 74:42-48; Huang et al. (1999) J. Virol. 73:2798-2802; Segerman et al. (2000) J. Virol. 74:1457-1467; Wu et al. (2001) Virology 279:78-89; Shayakhmetov et al. (2000) J. Virol. 74:2567-2584).

1. Fiber Protein

The adenovirus fiber protein is a homotrimeric protein containing three polypeptides of 62 kDa. Ad fiber proteins are located at each of the twelve icosahedral vertices of the viral particle (Chroboczek et al. (1995) Curr. Top. Microbiol. Immunol. 199:163-200). The sequences of the fiber gene from a variety of serotypes including adenovirus serotypes 2 (Ad2), Ad5, Ad3, Ad12, Ad35, Ad40, and Ad41 are known. There are at least 21 different fiber genes in Genbank. Sequence analysis of fiber proteins from several different adenovirus serotypes (Hong et al. (1988) Virology 167:545-553; Kidd et al. (1990) Virology 179:139-150; Signas et al. (1985) J. Virol. 53:672-678) and the crystal structure of Ad2 fiber (van Raaij et al. (1999) Nature 401:935-938) have identified three structural domains in the fiber. The N-terminal region of the fiber protein interacts with the penton base proteins to anchor the fiber to the viral particle. The C-terminal knob region is responsible for mediating virus binding to host cells. These two regions are connected via a long, thin central shaft region, which contains a variable number of shaft repeats, each repeat being made up of 15 residues designated as a-o. The repeating domains of the fiber shaft are characterized by an invariant glycine or proline at position j and a conserved pattern of hydrophobic residues (van Raaij et al. (1999) Nature 401:935-938). A conserved stretch of amino acids which includes the sequence TLWT (SEQ ID No. 46) marks the boundary between the repeating units of beta structure in the shaft and the globular head domain. The number of shaft repeats in Ad fiber depends on the adenoviral serotype. For example, Ad2 and Ad5 fiber proteins include 22 shaft repeats, while Ad3 contains only 5 repeats (Chroboczek et al. (1995) Curr. Top. Microbiol. Immunol. 199:163-200).

The C-terminal fiber knob mediates attachment to CAR, which is a 46 kDa protein of the immunoglobulin superfamily that is found on many different cell types (Bergelson et al. (1997) Science 275:1320-1323). A crystal structure of the Ad12 fiber in complex with CAR demonstrates that sequences in the fiber knob, specifically the AB loop, interact with the first Ig-like domain of CAR (Bewley et al. (1999) Science 286:1579-1583). Following attachment to CAR, binding of the Ad penton base protein to aV integrins enables internalization and penetration of the virus into the cell.

Adenovirus interactions with specific cell types are also influenced by the capacity to bind HSP. As noted, adenoviruses having fiber shafts that do not interact with HSP include (a) adenoviruses of subgroup B, e.g., Ad3, Ad35, Ad7, Ad11, Ad16, Ad21, (b) adenoviruses of subgroup F, e.g., Ad40 and Ad41, specifically the short fiber, and (c) adenoviruses of subgroup D, which includes adenovirus serotype 8, 9, 10, 13, 15, 17, 19, 20, 22-30, 32, 33, 36-39, and 42-49. Serotype 19 has variants. Ad 19p is a nonpathogenic variant of Ad19 (Arnberg et al. (1998) Virology 227:239-244) while Ad19a, along with Ad8 and Ad37, are major causes of EKC. Ad19a and Ad37 have identical fiber proteins (Arnberg et al. (1998) Virology 227:239-244) and have similar tropism in vivo.

2. Pseudotyping

Adenoviral vectors can be modified for targeting specific tissue and/or cell types through a variety of modifications, including modifications to the viral capsid, particularly to the fiber protein. Modifications provided herein include, but are not limited to, pseudotyping of the viral particle with heterologous and/or chimeric fiber protein.

Fibers that use non-CAR receptors can direct infection of a variety of different cell types (Shayakhmetov et al. (2000) J. Virol. 74:2567-2584; Von Seggern et al. (2000) J. Virol. 74:354-362; Law and Davidson (2002) J. Virol. 76:656-661; Havenga et al. (2002) J. Virol. 76:4612-4620; Gall et al. (1996) J. Virol. 70:2116-2123; Chillon et al. (1999) J. Virol. 73:2537-2540), thus providing a means for adenovirus vector targeting. Adenovirus packaging cell systems allow generation of viral particles with essentially any desired fiber protein by trans-complementation of a fiber-deleted virus (Von Seggern et al. (2000) J. Virol. 74:354-362; Von Seggern et al. (1999) J. Virol. 73:1601-1608). This technology, referred to as psuedotyping, allows generation of targeted viral particles that can be used to study tropism in vitro and in vivo, as well as permitting construction and propagation of viruses whose fibers do not bind to the producer cells normally used for Ad growth.

As described herein, psuedotyping can be used to identify fibers from subgroup D adenoviruses that confer enhanced infectivity of dendritic cells. Fiberless adenovirus vectors can be pseudotyped with fiber proteins from different serotypes to generate adenovirus particles with heterologous fiber proteins. Pseudotyping can be accomplished, for example, by expression in cells that contain expression plasmids encoding the fibers for pseudotyping. These vectors and plasmids can be generated as described herein or by any method known to those of skill in the art.

Accordingly, provided herein are modified fibers for targeting and detargeting and methods of making such fiber proteins and adenoviruses containing the fiber proteins. Among the cell types provided herein for adenovirus targeting are dendritic cells.

### C. DENDRITIC CELL TARGETING

Dendritic cells have numerous physiological features that render them desirable targets for immunotherapeutic approaches. Dendritic cells pick up antigens and migrate from the tissues of the body to the lymphoid tissues. There these cells present the antigens in lymphoid organs by displaying a foreign epitope bound to an MHC protein and trigger humoral and cellular immune responses. Dendritic cells have the ability to distinguish different types of pathogens, such as viruses, bacteria, fungi, and switch on specifically targeted immune-response genes against them. They are antigen-presenting cells that stimulate T lymphocytes into attacking infection. Hence delivery of heterologous antigens for presentation by dendritic cells provides a means for triggering humoral and cellular immune responses against such antigens. Also as noted, expression of particular products in dendritic cells also can function to inhibit or decrease in inappropriate or undesirable immune response, such occurs in allergies, autoimmune diseases and inflammatory responses.

1. Dendritic Cells

Dendritic cells (abbreviated DCs), which have a variety of important physiological features in the immune system, can serve as targets for immunotherapy and vaccine development. Dendritic cells play an important role in establishing an immune response. Dendritic cells are found in T-cell rich areas of the lymphoid tissues where they present antigen to T cells to stimulate the adaptive immune response (Janeway and Travers (1997) Immunobiology: the immune system in health and disease, third edition, Current Biology Ltd., New York, N.Y.).

As an antigen presenting cell, the role of the dendritic cell is to capture foreign and self antigens, process them into peptides, and present the peptides in the context of MHC (major histocompatibility complex) proteins to T lymphocytes. Dendritic cells are highly specialized and efficient APCs and they control the magnitude, quantity, and memory of the adaptive immune responses that they trigger (Steinman and Pope (2002) J. Clin. Invest. 109:1519-1526). The T cells activated by dendritic cells presenting antigen include, T-helper CD4+ cells, particularly cells designated Th1, and CD8+ cytotoxic T lymphocytes (CTLs). Activated Th1 cells produce IFN-γ and induce proliferation and antibody production of antigen-specific B lymphocytes. CTLs activated by dendritic cells kill cells displaying antigen (such as virus-infected cells) by releasing cytotoxic granules into the cell (see, e.g., Steinman and Pope (2002) J. Clin. Invest 109:1519-1526).

As noted, dendritic cells express high levels of MHC molecules for antigen presentation rendering them highly efficient APCs. In addition they also express a high level of co-stimulatory molecules, which are important for enhancing an immune response. Dendritic cells also produce a wide array of immunostimulatory cytokines (Scanlan and Jager (2001) Breast Cancer Res. 3:95-98) and there potentiate and participate in immune responses, and have been used as targets for vaccine development. Engineering of dendritic cells to express a tumor antigen has been pursued as an approach to tumor immunotherapy. For example, genetically modified dendritic cells that express particular antigens, such as tumor antigens, can be used as vaccines. In addition, genetic therapy that targets such cells in vivo can be used to generate APCs in vivo in immunotherapeutic methods.

Dendritic cells also are capable of diminishing an immune response. Dendritic cells can be exploited to aid in vaccination against autoimmunity, allergy and transplantation rejection, all of which result from an uncontrolled or unchecked immune response (Hawiger et al. (2001) J. Exp. Med. 194:769-779; Steinman et al. (2003) Annual Rev. Immunol. 21:685-711). For example, dendritic cells appear to be important for peripheral T cell tolerance (see, e.g., Steinman et al. (2000) J. Exp. Med. 191:411-416). Tolerance, or unresponsiveness to an antigen, is critical for avoidance of autoimmunity. Dendritic cells are capable of inducing significant antigen-specific tolerance in peripheral lymphoid tissues (Hawiger et al. (2001) J. Exp. Med. 194:769-779), and also are capable of inducing tolerance to transplantation antigens (see, Fu et al. (1996) Transplantation 62:659-665) and contact allergens (se, Steinbrink et al. (1997) J. Immunol. 159:4772-4780).

Thus, vaccine and immunotherapeutic strategies involving dendritic cells are important for the treatment of a variety of clinically important autoimmune and related diseases, including systemic lupus erythematosus, myasthenia gravis, rheumatoid arthritis, insulin-dependent diabetes mellitus and Graves' disease, as well as for vaccination or treatment of cancers and diseases caused by pathogens.

2. Dendritic Cell Therapies

Different methods for delivery of the antigen gene to dendritic cells have been explored, but these generally require ex vivo manipulation of cells, including transfection, and then infusion of the cells. This is complicated, expensive, and requires generation of patient-specific reagents.

Adenovirus can be used for dendritic cell therapies. The ability of adenovirus serotypes to infect specific cell types, such as dendritic cells, can in part be attributed to their interaction, or a lack of interaction, with CAR. For example, the requirement for high doses of Ad5 in dendritic cell (DC) transduction can be explained by the lack of CAR expression on dendritic cells (Linette et al. (2000) J. Immunol. 164:3402-3412; Tillman et al. (1999) J. Immunol. 162:6378-6383). Several approaches have been used to improve DC infection by adenoviruses. A bispecific antibody (Ab) which bound to the fiber knob as well as to CD40 (which is expressed on the surface of DCs) was used to target dendritic cells (Tillman et al. (1999) J. Immunol. 162:6378-6383). This study showed that the cells expressed sufficient αᵥ integrins for efficient infection by the DC-binding adenovirus. This approach requires that production of the viral vector and the antibody, and purification of the complex, in clinically acceptable forms. This presents problems with scale-up and manufacturing, and negates many of the advantages of adenovirus vectors, most notably simple production and purification, as well as vector stability.

Therefore to overcome these limitations, provided herein are adenoviral vectors that have been modified for efficiently targeting dendritic cells. The adenoviral vectors can be used for targeting such cells in vivo and ex vivo for immunotherapy and in vitro for studying dendritic cell function.

3. Targeting Adenoviral Particles to Dendritic Cells

Numerous studies have shown that adenovirus (Ad)-mediated delivery to dendritic cells can lead to anti-tumor response, but the Ad vectors generally used in gene therapy are based on a serotype (Ad5) that infects dendritic cells very inefficiently. After in vivo Ad administration, infection of a fairly small number of dendritic cells has been directly demonstrated (Zhang et al. (2001) Mol. Therapy. 3:697-707; Oberholzer et al. (2002) J. Immunol. 168:3412-3418; Jooss et al. (1998) J. Virol. 72:4212-4223) and appears to be largely responsible for the cellular immune response observed (Zhang et al. (2001) Mol. Therapy. 3:697-707; Jooss et al. (1998) J. Virol. 72:4212-4223). Since Ad5 infects dendritic cells poorly (Dietz et al. (1998) Blood 91:393-398; Wan et al. (1997) Human Gene Ther. 8:1355-1363; Jonuleit et al. (2000) Gene Therapy 7:249-254; Linette et al. (2000) J. Immunol. 164:3402-3412; Tillman et al. (1999) J. Immunol. 162:6378-6383) high multiplicities of infection are required.

Most testing has been done using primary cultures of dendritic cells derived from peripheral blood or bone marrow by incubation with cytokines, usually GM-CSF and IL-4 (Inaba et al. (1998) Isolation of dendritic cells In Current Protocols in Immunology, John Wiley & Sons, Inc. Philadelphia, 3.7.1-3.7.15). Ex vivo infection of dendritic cells, followed by re-infusion, has been found to generate effective anti-tumor responses (Wan et al. (1997) Human Gene Ther. 8:1355-1363; Linette et al. (2000) J. Immunol. 164:3402-3412; Inoue et al. (1999) Immunol. Lett. 70:77-81; Sonderbye et al. (1998) Exp. Clin. Immunogenet. 15:100-111; Ranieri et al. (1999) J. Virol. 73:10416-10425; Ribas et al. (1997) Cancer Res. 57:2865-2869; Miller et al. (2000) Human Gene Ther. 11:53-65). Ex vivo infection is not the ideal means for vaccination and immunotherapy.

In addition, recombinant adenoviruses with fiber proteins from the subgroup B viruses Ad16 and Ad35 have been found to have an increased ability to infect human dendritic cells (Havenga et al. (2002) J. Virol. 76:4612-4620; Rea et al. (2001) J. Immunol. 166:5236-5244). Subgroup B viruses, however, appear to have a broad tropism. For example, they transduce a wide variety of cultured cell lines as well as primary cells from a number of different tissue types (Havenga et al. (2002) J. Virol. 76:4612-4620), evidencing such broad tropism. This apparent lack of cell-specificity (broad tropism) demonstrated by subgroup B indicates that pseudotyping Ad5 or Ad2 viruses with Ad subgroup B fibers is not advantageous.

a. Fiber Substitution

It is shown herein that, contrary to reports in the literature, subgroup D viruses can target dendritic cells. Subgroup D viruses exhibit a narrower tropism than subgroup B viruses. It is shown herein that fibers from certain non CAR-using Ad serotypes, particularly Ad subgroup D viruses, effectively target receptors on dendritic cells.

Modified adenovirus particles can be generated by substituting dendritic cell-tropic fibers, such as the Subgroup D fibers, or portions thereof in place of the Ad subgroup C (or other Ad subgroup, including B and D, with a heterologous fiber), such as Ad5 or Ad2 fiber to produce detargeted (reduced binding to CAR, HSP) and retargeted (to dendritic cells) viral particles.

Any portion of the fiber can replaced with a portion of a subgroup D fiber, so long as the portion of the subgroup D fiber confers targeting to dendritic cells and the fiber assembles into the viral capsid. In one embodiment, the entire fiber protein is replaced with a subgroup D fiber. In another embodiment, the entire fiber, except for the N-terminus is replaced. For example, at least about 16 or 17 amino acids or more, up to about 60, 70, 80, 90, 100 or more amino acids of N-terminus of the native fiber is retained to aid in the incorporation of the fiber into the native particle.

Included in the modified adenoviruses provided herein are those with fiber protein from subgroup B and D, including, but not limited to Ad19p, Ad37, Ad30, Ad8, Ad9, Ad10, Ad13, Ad15, Ad17, Ad19, Ad20, Ad16, Ad35 and adenovirus serotypes 22-30, 32, 33, 36-39, and 42-49, expressed on adenoviral particles, particularly subgroup C particles. Among the modified capsid proteins are provided herein are those which include fibers containing the sequence of amino acids set forth in any of SEQ ID NOs. 32, 34, 36, 38 or 40; or a sequence of amino acids having 60%, 70%, 80%, 90%, 95% or greater sequence identity with a sequence of amino acids set forth in any of SEQ ID NOs. 32, 34, 36, 38 or 40; or a sequence of amino acids encoded by a sequence of nucleotides that hybridizes under conditions of high stringency along at least 70%, at least 80% or at least 90% of its length to a sequence of nucleotides that encodes a sequence of amino acids set forth in any of SEQ ID NOs. 32, 34, 36, 38 or 40. The fiber proteins can be modified, such as described herein, by replacement of the N-terminus to facilitate incorporation into the viral particle of a different subgroup, particularly subgroup C. Such modification is generally inclusion of at least 16 or 17 amino acids up to about 60 or 61 or more contiguous amino acids from the N-terminus of the native fiber such that dendritic cell targeting is introduced.

In one exemplary embodiment, a packaging cell strategy is used to produce particles of a fiber-deleted Ad5 vector containing fiber proteins from Ads of subgroup B (Ad3, Ad16, Ad35), subgroup C (Ad5), and subgroup D (Ad19p, Ad30, Ad37). Nucleotide and amino acid sequences of Ad fibers are set forth in SEQ ID NOs. 41-44 (exemplary chimeric fibers) and 31-40 (exemplary wild type fibers that can be modified by replacement of the N-terminus). The resulting particles exhibit significant differences in dendritic cells tropism as demonstrated by their ability to infect primary murine bone marrow-derived DC in vitro. Furthermore, the particles pseudotyped with the subgroup D particles efficiently and specifically target dendritic cells. As described in the herein (see e.g., the Examples) subgroup B fibers appear to bind to receptors distinct from and more ubiquitously expressed than those bound by subgroup D fibers.

While particles with the Ad5 fiber infect dendritic cells rather poorly, vector particles pseudotyped with subgroup D fibers or portions thereof, such as the Ad19p and Ad37, were particularly effective. Thus, adenovirus particles, particularly subgroup C particles, modified to express all or a portion of a subgroup D fiber, efficiently target dendritic cells and can be used to deliver heterologous nucleic acids to such cells in vivo and ex vivo. In addition, such particles have reduced binding to HSP-expressing cells, such as hepatocytes and to CAR-expressing cells compared to unmodified subgroup B viral particles.

b. Efficient Targeting

Provided herein are recombinant adenoviruses with a limited tropism that target dendritic cells. The recombinant adenoviruses can be used for gene therapy and/or vaccination approaches. Administration can be effected in vivo, such as systemically, or ex vivo by contacting cells enriched for or containing dendritic cells.

The recombinant adenoviruses provided herein have a variety of advantageous properties. The particles provided herein more efficiently infect dendritic cells than Ad5 particles or Ad5 particles that express subgroup B fibers, and hence are more immunogenic following direct in vivo administration. The vectors provided herein that efficiently target dendritic cells permit not only ex vivo delivery, but direct in vivo administration, thereby eliminating the need for removal of cells, ex vivo cell culture, and infusion.

4. Additional Modifications

The modified adenoviruses provided herein not only exhibit improved tropism for dendritic cells, but also reduced binding to HSP, which is expressed on liver cells. The modified particles can be further modified to be detargeted from CAR, HSP, aᵥ integrin, or any other native receptors, by any of the capsid mutations described below or well known to those of skill in the art.

The vectors provided herein also can be modified by including a RGD peptide in the fiber protein. It has been shown (see, e.g., Okada et al. (Cancer Res. 61:7913-7919 (2001)) that incorporation of an RGD peptide into the fiber protein increased infection of a murine DC line approximately two-fold. The cell line/Ad system was then used to evaluate anti-tumor responses in a mouse tumor xenograft model. When DCs were infected ex vivo using equal particle numbers of the wild type or modified vectors and then re-infused into mice, the modified vector was able to stimulate a significantly better immune response against the model antigen.

The particles provided herein also can be further modified by inclusion of heterologous nucleic acid that provides a therapeutic product, and formulated for administration as vaccines. The adenovirus particles and vectors can deliver heterologous nucleic acids to dendritic cells to alter dendritic cell antigen presentation, cytokine production and other dendritic cell functions.

### D. ADENOVIRUS VECTOR DETARGETING

Described below are modifications of the viral capsid that ablate the interaction of an adenovirus with its natural receptors. In particular, fiber modifications that result in ablation of the interaction of an adenovirus with HSP are described. These fiber modifications can be combined with other capsid protein modifications, such as other fiber modifications and/or penton and/or hexon modifications, to fully ablate viral interactions with natural receptors, when expressed on a viral particle. The modification should not disrupt trimer formation or transport of fiber into the nucleus. The entire fiber of a serotype that binds to HSP can be replaced with all or a portion of a fiber that does not bind to HSP. Generally in such instances, the N-terminus of the replacing fiber is modified to resemble or to be identical to the replaced fiber to improve its incorporation into the viral particle. The number of amino acids at the N-terminus required can be empirically determined, but is typically between about 5-20, 10-17, 10-20, 10-50, 10-70, 10-100, amino acids, more amino acids can be included if convenient. The precise number also can be based upon the presence of convenient restriction sites in the encoding nucleic acid and other such considerations. Generally at least about 5-20, such as 16, 17, or 18, amino acids are required.

The adenovirus fiber protein is a major determinant of adenovirus tropism (Gall et al. (1996) J. Virol. 70:2116-2123; Stevenson et al. (1995) J. Virol. 69:2850-2857). Dogma in the field has been that adenoviral entry occurs via binding to CAR and integrins. This is underscored by published data (Einfeld et al. (2001) J. Virology 75:11284-11291). The published are not the predominant ones that act in vivo. The dominant entry pathway for hepatocytes in vivo involves a mechanism mediated by the fiber shaft, such as Ad5 shaft, through heparin sulfate proteoglycans binding (see, published U.S. application Nos. 2004-0002060 and 2003-0215948).

Elimination of this binding eliminates entry via HSP binding, such as in hepatocytes. Adenoviral fiber shaft modifications that ablate viral interaction with HSP are described in the Examples below and in published U.S. application Nos. 2004-0002060 and 2003-0215948. Thus, efficient detargeting of adenovirus in vivo can be achieved with appropriately designed fiber proteins. Suitable modifications, such as described herein, can be made with respect to any adenovirus in which the wild-type interacts with HSP. The ability of an adenoviral vector to interact with HSP is modified by replacing the fiber protein or at least the binding portion thereof with a fiber (or corresponding portion thereof) that does not bind to HSP thereby reducing or eliminating binding to HSP. This reduction or elimination of HSP binding can be manifested in vivo as reduced or eliminated transduction of liver cells in animals to whom the resulting viral particles are administered compared to the unmodified particle. Modifications include insertions, deletions, individual amino acid mutations and other mutations that alter the structure of the fiber shaft such that the HSP binding of the modified fiber protein is ablated when compared to the HSP binding of the wild-type fiber protein.

An adenoviral fiber protein is modified by mutating one or more of the amino acids that interact with HSP. For example, the HSP binding motif of the modified fiber protein is no longer able to interact with HSP on the cell surface, thus ablating the viral interaction with HSP. For example, the adenoviral fiber is from a subgroup C adenovirus. Binding to HSP can be eliminated or reduced by mutating the fiber shaft in order to modify the ability of the HSP binding motif, which is, for example, KKTK sequence (SEQ ID NO. 45) located between amino acid residues 91 to 94 in the Ad5 fiber (SEQ ID NO. 2), to interact with HSP. The fiber proteins are modified by chemical and biological techniques known to those skilled in the art, such as site directed mutagenesis of nucleic acid encoding the fiber or other techniques as illustrated herein.

In another aspect of this embodiment, the ability of a fiber to interact with HSP is modified by replacing the wild-type fiber shaft with a fiber shaft, or portion thereof, of an adenovirus that does not interact with HSP to produce chimeric fiber proteins. The portion is sufficient to reduce or eliminate interaction with HSP. Examples of adenoviruses having fiber shafts that do not interact with HSP include (a) adenoviruses of subgroup B, such as, but are not limited to, Ad3, Ad7, Ad11, Ad16, Ad21, Ad34 and Ad35 which do not have interaction with HSP, (b) adenoviruses of subgroup F, such as, but are not limited to, Ad40 and Ad41, specifically the short fiber, and (c) adenoviruses of subgroup D, such as but are not limited to, Ad19p, Ad30, Ad37 and Ad46.

In another embodiment, adenoviral fiber shaft modifications and/or pseudotyped fibers that ablate viral interaction with HSP in combination with adenoviral fiber knob modifications that ablate viral interactions with CAR are provided. Suitable adenoviral fiber modifications include the fiber knob modifications described in the Examples below and modifications known to those of skill in the art (see published U.S. application Nos. 2004-002060 and 2003-0215948; see, also, U.S. patent application Ser. No. 09/870,203, filed on May 30, 2001, published as U.S. Published application No. 20020137213, and International Patent Application No. PCT/EP01/06286, filed on Jun. 1, 2001, published as WO 01/92299). Modifications of the fiber include mutations of at least one amino acid in the CD loop of a wild-type fiber protein of an adenovirus from subgroup C (such as, e.g., Ad2 or Ad5), subgroup D (such as, e.g., Ad19p, Ad30 or Ad37), subgroup E, or the long wild-type fiber of an adenovirus from subgroup F, whereby the ability of a fiber protein to bind to CAR is reduced or substantially eliminated. The fiber proteins with ablated CAR interaction are modified by chemical and biological techniques known to those skilled in the art and as described herein.

Alternatively, adenoviral fiber modifications are made by replacing the wild-type fiber knob with a fiber knob of an adenovirus that does not interact with CAR. The fiber protein also will be selected so that it does not interact with HSP. Examples of adenoviruses having fiber knobs that do not interact with CAR include (a) adenoviruses of subgroup B, e.g., Ad3, Ad7, Ad11, Ad16, Ad21, Ad34, Ad35; and (b) adenoviruses of subgroup F, e.g., Ad40 and Ad41, specifically the short fiber.

In another embodiment, adenoviral fiber shaft modifications and/or pseudotyped fibers that ablate viral interaction with HSP in combination with penton modifications that ablate viral interactions with αᵥ integrins are provided. Suitable adenoviral penton modifications include the penton modifications, which are well known to those of skill in the art (see, e.g., U.S. Pat. No. 5,731,190; see, also Einfeld et al. (2001) J. Virology 75:11284-11291; and Bai et al. (1993) J. Virology 67:5198-5205).

For example, penton interaction with αᵥ integrins can be ablated (reduced or eliminated) by substitution of the RGD tripeptide motif, required for aV interaction, in penton with a different tripeptide that does not interact with an αᵥ integrin. The penton proteins with ablated αᵥ integrin interactions are modified by chemical and biological techniques known to those skilled in the art (see, e.g., described U.S. Pat. No. 6,731,190 and as illustrated herein).

Also provided are adenoviral fiber shaft modifications or pseudotyped fibers that ablate viral interaction with HSP in combination with adenoviral fiber knob modifications that ablate viral interactions with CAR and with penton modifications that ablate viral interactions with αᵥ integrins. These modifications are described above and prepared using chemical and biological techniques known to those skilled in the art and as illustrated herein.

Preparation of fibers modified to eliminate or reduce HSP interactions and fibers modified to alter interactions with other receptors and cell surface proteins, such as CAR and/or αᵥ integrin, also is described in the Examples below. The nucleic acid and/or amino acid sequences of exemplary modified fibers, whose construction are described below) are set forth as SEQ ID NOs. 3-30 as follows:

SEQ ID NOs. 3 and 4 set forth the encoding nucleotide sequence and amino acid sequence of the modified fiber designated 5FKO1, where 5F refers to adenovirus 5 fiber, KO1 is an exemplary mutation of the CAR interaction site described herein;

SEQ ID NOs. 5 and 6 set forth the encoding nucleotide sequence and amino acid sequence of the modified fiber designated 5FKO1RGD, which further includes an RGD ligand to demonstrate retargeting;

SEQ ID NOs. 7 and 8 set forth the encoding nucleotide sequence and amino acid sequence of the modified fiber designated 5FKO12, where 5F refers to adenovirus 5 fiber, KO12 is another exemplary mutation of the CAR interaction site described herein;

SEQ ID NOs. 9 and 10 set forth the encoding nucleotide sequence and amino acid sequence of the modified fiber designated 5F S* nuc, where 5F refers to adenovirus 5 fiber, S* is an exemplary mutation of the shaft that alters binding to HSP;

SEQ ID NOs. 11 and 12 set forth the encoding nucleotide sequence and amino acid sequence of the modified fiber designated 5F S*RGD nuc, which further includes an RGD ligand;

SEQ ID NOs. 13 and 14 set forth the encoding nucleotide sequence and amino acid sequence of the modified ber designated 5FKO1S*, which contain the KO1 and S* mutations;

SEQ ID NOs. 15 and 16 set forth the encoding nucleotide sequence and amino acid sequence of the modified fiber designated 5FKO1S*RGD, which further includes an RGD ligand;

SEQ ID NOs. 17 and 18 set forth the encoding nucleotide sequence and amino acid sequence of a Ad35 fiber;

SEQ ID NOs. 19 and 20 set forth the encoding nucleotide sequence and amino acid sequence of the modified fiber designated 35FRGD, which is 35F fiber with an RGD ligand;

SEQ ID NOs. 21 and 22 set forth the encoding nucleotide sequence and amino acid sequence of a Ad41 short fiber;

SEQ ID NOs. 23 and 24 set forth the encoding nucleotide sequence and amino acid sequence of the modified fiber designated 41sFRGD, which is 41F short fiber with an RGD ligand;

SEQ ID NOs. 25 and 26 set forth the encoding nucleotide sequence and amino acid sequence of Ad5 penton;

SEQ ID NOs. 27 and 28 set forth the encoding nucleotide sequence and amino acid sequence of the modified fiber designated 5TS35H, which is a chimeric fiber in which an Ad5 fiber tail and shaft regions (5TS; amino acids 1 to 403) are connected to an Ad35 fiber head region (35H; amino acids 137 to 323) to form the 5TS35H chimera; and

SEQ ID NOs. 29 and 30 set forth the encoding nucleotide sequence and amino acid sequence of the modified fiber designated 35TS5H, which is a chimeric fiber in which an Ad35 fiber tail and shaft regions (35TS; amino acids 1 to 136) are connected to an Ad5 fiber head region (5H; amino acids 404 to 581) to form the 35TS5H chimera.

The modified fibers are displayed on virus particles by modifying the fiber protein and optionally additional proteins. This can be achieved by preparing adenoviral vectors that express the modified capsid proteins and produce particles with modified fibers, or by packaging adenoviral vectors, particularly those that do not encode one or more capsid proteins in appropriate packaging lines. Hence, as discussed in detail below, adenoviral vectors and viral particles with modified fibers that do not bind to HSP are provided.

Retargeting Detargeted Fibers

The viral particles that are detargeted as described above, can be retargeted to selected cells and/or tissues by inclusion of an appropriate targeting ligand in the capsid. The ligand can be included in any of the capsid proteins, such as fiber, hexon and penton. Loci for inclusion of nucleic acid encoding a targeting ligand is known to those of skill in the art for a variety of adenovirus serotypes; if necessary appropriate loci and other parameters can be empirically determined.

The ligand can be produced as a fusion by inclusion of the coding sequences in the nucleic acid encoding a capsid protein, or chemically conjugated, such as via ionic, covalent or other interactions, to the capsid or bound to the capsid (e.g., by Ab-ligand fusion, where Ab binds capsid protein; or by disulfide bonding or other crosslinking moieties or chemistries).

Thus, for example, a modified fiber nucleic acid also can include sequences of nucleotides that encode a targeting ligand to produce viral particles that include a targeting ligand in the capsid. Targeting ligand and methods for including such ligands in viral capsid are well known. For example, inclusion of targeting ligands in fiber proteins is described in U.S. Pat. Nos. 5,543,328 and 5,756,086 and in U.S. patent application Ser. No. 09/870,203, published as U.S. Published application No. 20020137213, and International Patent Application No. PCT/EP01/06286, published as WO 01/92299. For different serotypes and strains of adenoviruses, loci for insertion of targeting ligands can be empirically determined. For different serotypes and strains, such loci can vary.

Because the adenovirus fiber has a trimeric structure, the ligand can be selected or designed to have a trimeric structure so that up to three molecules of the ligand are present for each mature fiber. Such ligands can be incorporated into the fiber protein using methods known in the art (see, e.g., U.S. Pat. No. 5,756,086). Instead of the fiber, the targeting ligand can be included in the penton or hexon proteins. Inclusion of targeting ligands in penton (see for example, in U.S. Pat. Nos. 5,731,190 and 5,965,431) and in hexon (see for example, in U.S. Pat. No. 5,965,541) is known.

In one exemplary embodiment, the ligand is included in a fiber protein, which is a fiber protein mutated as described herein. The targeting ligand can be included, for example, within the HI loop of the fiber protein. Any ligand that can fit in the HI loop and still provide a functional virus is contemplated herein. Such ligands can be as long as or longer than 80-100 amino acids (see, e.g., Belousova et al. (2002) J. Virol. 76:8621-8631). Such ligands are added by techniques known in the art (see, e.g., published International Patent Application publication No. WO 99/39734 and U.S. patent application Ser. No. 09/482,682). Other ligands can be discovered through techniques known to those skilled in the art. Some non-limiting examples of these techniques include phage display libraries or by screening other types of libraries. Such ligands include any that target dendritic cells.

Targeting ligands include any chemical moiety that preferentially directs an adenoviral particle to a desired cell type and/or tissue, such as a dendritic cell. The categories of such ligands include, but are not limited to, peptides, polypeptides, single chain antibodies, and multimeric proteins. Specific ligands include the TNF superfamily of ligands which include tumor necrosis factors (or TNF's) such as, for example, TNF-α and TNF-β, lymphotoxins (LT), such as LT-α and LT-β, Fas ligand which binds to Fas antigen; CD40 ligand, which binds to the CD40 receptor of B-lymphocytes; CD30 ligand, which binds to the CD30 receptor of neoplastic cells of Hodgkin's lymphoma; CD27 ligand, NGF ligand, and OX-40 ligand; transferrin, which binds to the transferrin receptor located on tumor cells, activated T-cells, and neural tissue cells; ApoB, which binds to the LDL receptor of liver cells; alpha-2-macroglobulin, which binds to the LRP receptor of liver cells; alpha-I acid glycoprotein, which binds to the asialoglycoprotein receptor of liver; mannose-containing peptides, which bind to the mannose receptor of macrophages; sialyl-Lewis-X antigen-containing peptides, which bind to the ELAM-1 receptor of activated endothelial cells; CD34 ligand, which binds to the CD34 receptor of hematopoietic progenitor cells; ICAM-I, which binds to the LFA-I (CD11b/CD18) receptor of lymphocytes, or to the Mac-I (CD11a/CD18) receptor of macrophages; M-CSF, which binds to the c-fms receptor of spleen and bone marrow macrophages; circumsporozoite protein, which binds to hepatic Plasmodium falciparum receptor of liver cells; VLA-4, which binds to the VCAM-1 receptor of activated endothelial cells; HIV gp120 and Class II MHC antigen, which bind to the CD4 receptor of T-helper cells; the LDL receptor binding region of the apolipoprotein E (ApoE) molecule; colony stimulating factor, or CSF, which binds to the CSF receptor; insulin-like growth factors, such as IGF-I and IGF-II, which bind to the IGF-I and IGF-II receptors, respectively; Interleukins 1 through 14, which bind to the Interleukin 1 through 14 receptors, respectively; the Fv antigen-binding domain of an immunoglobulin; gelatinase (MMP) inhibitor; bombesin, gastrin-releasing peptide; substance P; somatostatin; luteinizing hormone releasing hormone (LHRH); vasoactive peptide (VIP); gastrin; melanocyte stimulating hormone (MSH); cyclic RGD peptide and any other ligand or cell surface protein-binding (or targeting) molecule. Such ligands can be advantageously employed with the Ad5 particles pseudotyped with subgroup D adenovirus fiber, such as, for example, Ad19p, Ad30 or Ad37 fiber.

### E. NUCLEIC ACIDS, ADENOVIRAL VECTORS AND CELLS CONTAINING THE NUCLEIC ACIDS AND CELLS CONTAINING THE VECTORS

Also provided are polynucleotides that encode modified, including chimeric and/or heterologous, capsid proteins and that encode vectors for preparation of adenovirus that express modified capsid proteins provided herein. The sequences of the wild-type adenovirus proteins from many different adenovirus serotypes are well known in the art and are modified as described herein or by any suitable method.

Also provided are vectors including the polynucleotides provided herein. Such vectors include partial or complete adenoviral genomes and plasmids. Such vectors are constructed by techniques known to those skilled in the art and as illustrated herein. Also provided are adenoviral vectors modified by replacing whole fiber protein, or portions thereof, with the fiber proteins, or appropriate portions thereof, from an adenovirus of a different serotype that more efficiently targets dendritic cells. Adenoviruses that target dendritic cells can be identified by using the methods described herein. Their fiber-encoding genes can then be used to pseudotype viruses, such as Ad5 or Ad2 and infection and gene delivery of adenoviruses with the heterologous or chimeric fibers can be detected. Among the adenoviral vectors provided herein are those of subgroup C, which include Ad2 and Ad5, in which the nucleic acid encoding the fiber knob and a portion or all of the fiber shaft domain is replaced with nucleic acid encoding fiber or an appropriate portion thereof from a subgroup D adenovirus, such as Ad19p, Ad30 or Ad37.

Thus, adenoviral fiber modifications or substitutions can be made in viral particles by replacing the entire fiber protein, or a portion thereof, with the fiber protein of an adenovirus that more efficiently binds to receptors on dendritic cells. Generally the heterologous adenovirus fiber is from a subgroup D adenovirus, such as Ad19p, Ad30 or Ad37. Adenoviral vectors of subgroup C, such as Ad2 and Ad5, having a replaced fiber knob are prepared using techniques well known in the art and as illustrated herein.

In particular, as exemplified herein, the nucleic acid and/or amino acid sequences of exemplary heterologous and/or modified fibers for dendritic cell targeting are set forth as SEQ ID NOs. 31-44 as follows:

SEQ ID NOs. 31 and 32 set forth the encoding nucleotide sequence and amino acid sequence of Ad37 fiber.

SEQ ID NOs. 33 and 34 set forth the encoding nucleotide sequence and amino acid sequence of Ad19p fiber.

SEQ ID NOs. 35 and 36 set forth the encoding nucleotide sequence and amino acid sequence of Ad30 fiber.

SEQ ID NOs. 37 and 38 set forth the encoding nucleotide sequence and amino acid sequence of Ad16 fiber.

SEQ ID NOs. 39 and 40 set forth the encoding nucleotide sequence and amino acid sequence of Ad35 fiber.

SEQ ID NOs. 41 and 42 set forth the encoding nucleotide sequence and amino acid sequence of Ad5/Ad16 chimeric fiber. The chimeric fiber contains the N-terminal 17 amino acids from Ad5 and the remainder of the sequence is from Ad16.

SEQ ID NOs. 43 and 44 set forth the encoding nucleotide sequence and amino acid sequence of Ad5/Ad35 chimeric fiber. The chimeric fiber contains the N-terminal 17 amino acids from Ad5 and the remainder of the sequence is from Ad35.

1. Preparation of Viral Particles

The packaging cells used to produce the viruses provided herein contain the nucleic acid encoding the capsid (i.e. fiber, penton, hexon) protein. Such nucleic acid can be transfected into the cell, generally as part of a plasmid, or it can be infected into the cell with a viral vector. It can be stably incorporated into the genome of the cell, thus providing for a stable cell line. Alternatively, nucleic acid encoding the heterologous or mutated capsid protein can be removed from the genome, in which case a transient complementing cell is employed.

The adenovirus genome to be packaged is transferred into the complementing cell by techniques known to those skilled in the art. These techniques include transfection or infection with the adenovirus. The nucleic acid encoding the mutated or heterologous fiber protein can be in this genome instead of in the packaging cell.

In certain cases, when the nucleic acid in the genome to be packaged encodes a mutated or heterologous fiber protein, it can be desirable for the packaging cell to also encode a fiber protein. Such protein can assist in the maturation and packaging of an infectious particle. Such protein can be a wild-type fiber protein or one modified such that it is unable to attach to the penton base protein and is for use, for example, in producer cells where the fiber is included to provide the packaging function and the vector encodes a full-length fiber.

The packaging cells are cultured under conditions that permit the production of the desired viral particle. The viral particles are recovered by standard techniques. An exemplary method for producing adenoviral particles provided herein is as follows. The nucleic acid encoding the mutated or heterologous capsid protein is made using standard techniques in an adenoviral shuttle plasmid. This plasmid contains the right end of the virus, in particular from the end of the E3 region through the right ITR. This plasmid is co-transfected into competent cells of an E. coli strain, such as the well known E. coli strain BJ5183 (see, e.g., Degryse (1996) Gene 170:45-50) along with a plasmid, which contains the remaining portion of the adenovirus genome, except for the E1 region and sometimes also the E2a region and also contains a corresponding region of homology. Homologous recombination between the two plasmids generates a full-length plasmid encoding the entire adenoviral vector genome.

This full-length adenoviral vector genome plasmid is then transfected into a complementing cell line. The transfection can be performed in the presence of a reagent that directs adenoviral particle entry into producer cells. Such reagents include, but are not limited to, polycations and bifunctional reagents, such as those described herein. A complementing cell, for example, is a cell of the PER.C6 cell line, which contains the adenoviral E1 gene (PER.C6 is available, for example, from Crucell, The Netherlands; deposited under ECACC accession no. 96022940; see, also Fallaux et al. (1998) Hum. Gene Ther. 9:1909-1907; see, also, U.S. Pat. No. 5,994,128) or an AE1-2a cell (see, Gorziglia et al. (1996) J. Virology 70:4173-4178; and Von Seggern et al. (1998) J. Gen. Virol. 79:1461-1468)).

AE1-2a cells are derivatives of the A549 lung carcinoma line (ATCC # CCL 185) with chromosomal insertions of the plasmids pGRE5-2.E1 (also referred to as GRE5-E1-SV40-Hygro construct and listed in SEQ ID NO. 47) and pMNeoE2a-3.1 (also referred to as MMTV-E2a-SV40-Neo construct and listed in SEQ ID NO. 48), which provide complementation of the adenoviral E1 and E2a functions, respectively.

The 633 cell line (see, von Seggern et al. (2000) J. Virology 74:354-362), which stably expresses the adenovirus serotype 5 wild-type fiber protein, and was derived from the AE1-2a cell line, is another example of complementing cells. When the cell line is 633 cell line, the final passage of the adenoviral vector is performed on another complementing cell line (e.g., Per.C6), which does not express wild-type Ad5 fiber.

The transfected complementing cells are maintained under standard cell culture conditions. The adenoviral plasmids recombine to form the adenoviral genome that is packaged. The particles are infectious, but replication deficient because their genome is missing at least the E1 genes. When performed in the 633 cells the particles contain wild-type and mutated or heterologous fiber proteins. They are recovered from the crude viral lysate, amplified, and are purified by standard techniques.

The recovered particles can be used to infect PER.C6 or AE1-2a cells. This permits the recovery of particles whose capsids contain only the desired mutated fiber. This two-step procedure provides high titer batches of the adenoviral particles provided herein. The adenoviral particles can be replication competent or replication incompetent.

In one embodiment, the particles selectively replicate in certain predetermined target tissue but are replication incompetent in other cells and tissues. In a particular embodiment, the adenoviral particles replicate in abnormally proliferating tissue, such as solid tumors and other neoplasms. In replication conditional adenoviruses, a gene essential for replication is placed under control of a heterologous promoter which is cell or tissue specific. For example, the E1a gene is placed under control of a promoter which is active in a tumor cell to produce an oncolytic adenovirus or oncolytic adenoviral vector. Administration of oncolytic adenoviral vectors to tumor cells kills the tumor cells. Such replication conditional adenoviral particles and vectors can be produced by techniques known to those skilled in the art, such as those disclosed in the above-referenced U.S. Pat. Nos. 5,998,205 and 5,801,029. These particles and vectors can be produced in adenoviral packaging cells as disclosed above. Generally packaging cells are those that have been designed to limit homologous recombination that could lead to wild-type adenoviral particles. Such cells are well known and include the packaging cell known as PER.C6 (see, e.g., U.S. Pat. Nos. 5,994,128 and 6,033,908; deposited under ECACC accession no. 96022940).

2. Adenoviral Vectors and Particles

The adenovirus as used herein for production of the adenoviral vectors and particles can be of any serotype, such as an Ad5 or Ad2. Adenoviral stocks that can be employed as a source of adenovirus or adenoviral coat protein, such as fiber and/or penton base, can be amplified from the adenoviral serotypes 1 through 51, which are currently available from the American Type Culture Collection (ATCC, Rockville, Md.), or from any other serotype of adenovirus available from any other source. For instance, an adenovirus can be of subgroup A (e.g., serotypes 12, 18, 31), subgroup B (e.g., serotypes 3, 7, 11, 14, 16, 21, 34, 35, 50), subgroup C (e.g., serotypes 1, 2, 5, 6), subgroup D (e.g., serotypes 8, 9, 10, 13, 15, 17, 19, 20, 22-30, 32, 33, 36-39, 42-49, 51), subgroup E (serotype 4), subgroup F (serotype 40, 41), or any other adenoviral serotype. In certain embodiments, the adenovirus is a subgroup C adenovirus. Subgroup C adenoviruses which are modified in as described herein, include, but are not limited to, Ad2 and Ad5.

The adenoviral vectors provided herein can be used to study cell transduction and gene expression in vitro or in various animal models. The latter case includes ex vivo techniques, in which cells are transduced in vitro and then administered to the animal. They also can be used to conduct gene therapy on humans or other animals. Such gene therapy can be ex vivo or in vivo. For in vivo gene therapy, the adenoviral particles in a pharmaceutically-acceptable carrier are delivered to a human in a therapeutically effective amount in order to prevent, treat, or ameliorate a disease or other medical condition in the human through the introduction of a heterologous gene that encodes a therapeutic protein into cells in such human. The adenoviruses are delivered at a dose ranging from approximately 1 particle per kilogram of body weight to approximately 10¹⁴ particles per kilogram of body weight. Generally, they are delivered at a dose of approximately 10⁶ particles per kilogram of body weight to approximately 10¹³ particles per kilogram of body weight, and typically the dose ranges from approximately 10⁸ particles per kilogram of body weight to approximately 10¹² particles per kilogram of body weight.

Any vectors known to those of skill in the art can be employed and used to produce viral particles that include fibers modified to enhance binding and infectivity of dendritic cells.

a. Gutless Vectors

Gutted adenovirus vectors are those from which most or all viral genes have been deleted. They are grown by co-infection of the producing cells with a “helper” virus (such as using an E1-deleted Ad vector), where the packaging cells express the E1 gene products. The helper virus trans-complements the missing Ad functions, including production of the viral structural proteins needed for particle assembly. To incorporate the capsid modifications into a gutted adenoviral vector capsid, the changes must be made to the helper virus as described herein. All the necessary Ad proteins including the modified capsid protein are provided by the modified helper virus, and the gutted adenovirus particles are equipped with the particular modified capsid expressed by the host cells. The E1a, Eb, E2a, E2b and E4 are generally required for viral replication and packaging. If these genes are deleted, then the packaging cell must provide these genes or functional equivalents.

A helper adenovirus vector genome and a gutless adenoviral vector genome are delivered to packaging cells. The cells are maintained under standard cell maintenance or growth conditions, whereby the helper vector genome and the packaging cell together provide the complementing proteins for the packaging of the adenoviral vector particle. Such gutless adenoviral vector particles are recovered by standard techniques. The helper vector genome can be delivered in the form of a plasmid or similar construct by standard transfection techniques, or it can be delivered through infection by a viral particle containing the genome. Such viral particle is commonly called a helper virus. Similarly, the gutless adenoviral vector genome can be delivered to the cell by transfection or viral infection.

The helper virus genome can be the modified adenovirus vector genome as disclosed herein. Such genome also can be prepared or designed so that it lacks the genes encoding the adenovirus E1A and E1B proteins. In addition, the genome can further lack the adenovirus genes encoding the adenovirus E3 proteins. Alternatively, the genes encoding such proteins can be present but mutated so that they do not encode functional E1A, E1B and E3 proteins. Furthermore, such vector genome can not encode other functional early proteins, such as E2A, E2B3, and E4 proteins. Alternatively, the genes encoding such other early proteins can be present but mutated so that they do not encode functional proteins.

In producing the gutless vectors, the helper virus genome also is packaged, thereby producing helper virus. In order to minimize the amount of helper virus produced and maximize the amount of gutless vector particles produced, the packaging sequence in the helper virus genome can be deleted or otherwise modified so that packaging of the helper virus genome is prevented or limited. Since the gutless vector genome will have an unmodified packaging sequence, it will be preferentially packaged.

One way to do this is to mutate the packaging sequence by deleting one or more of the nucleotides comprising the sequence or otherwise mutating the sequence to inactivate or hamper the packaging function. One exemplary approach is to engineer the helper genome so that recombinase target sites flank the packaging sequence and to provide a recombinase in the packaging cell. The action of recombinase on such sites results in the removal of the packaging sequence from the helper virus genome. The recombinase can be provided by a nucleotide sequence in the packaging cell that encodes the recombinase. Such sequence can be stably integrated into the genome of the packaging cell. Various kinds of recombinase are known by those skilled in the art, and include, but are not limited to, Cre recombinase, which operates on so-called lox sites, which are engineered on either side of the packaging sequence as discussed above (see, e.g., U.S. Pat. Nos. 5,919,676, 6,080,569 and 5,919,676; see, also, e.g., Morsy and Caskey, Molecular Medicine Today, January 1999, pgs. 18-24).

An example of a gutless vector is pAdARSVDys (Haecker et al. (1996) Hum Gene Ther. 7:1907-1914)). This plasmid contains a full-length human dystrophin cDNA driven by the RSV promoter and flanked by Ad inverted terminal repeats and packaging signals. 293 cells are infected with a first-generation Ad, which serves as a helper virus, and then transfected with purified pAdARSVDys DNA. The helper Ad genome and the pAdARSVDys DNA are replicated as Ad chromosomes, and packaged into particles using the viral proteins produced by the helper virus. Particles are isolated and the pAdARSVDys-containing particles separated from the helper by virtue of their smaller genome size and therefore different density on CsCl gradients. Other examples of gutless adenoviral vectors are known (see, e.g., Sandig et al. (2000) Proc. Natl. Acad. Sci. U.S.A. 97(3):1002-7).

b. Oncolytic Vectors

Oncolytic adenoviruses are viruses that replicate selectively in tumor cells. Such vectors generally will not be useful for targeting dendritic cells, unless such cells are malignant. Briefly, oncolytic vectors are designed to amplify the input virus dose due to viral replication in the tumor, leading to spread of the virus throughout the tumor mass. In situ replication of adenoviruses leads to cell lysis. This in situ replication permits relatively low, non-toxic doses to be highly effective in the selective elimination of tumor cells. One approach to achieving selectivity is to introduce loss-of-function mutations in viral genes that are essential for growth in non-target cells but not in tumor cells (see, e.g., U.S. Pat. No. 5,801,029). This strategy is exemplified by the use of Addl1520, which has a deletion in the E1b-55 KD gene. In normal cells, the adenoviral E1b-55KD protein is needed to bind to p53 to prevent apoptosis. In p53-deficient tumor cells, E1b-55K binding to p53 is unnecessary. Thus, deletion of E1b-55KD should restrict vector replication to p53-deficient tumor cells.

Another approach is to use tumor-selective promoters to control the expression of early viral genes required for replication (see, e.g., International PCT application Nos. WO 96/17053 and WO 99/25860). Thus, in this approach the adenoviruses selectively replicate and lyse tumor cells if the gene that is essential for replication is under the control of a promoter or other transcriptional regulatory element that is tumor-selective.

For example oncolytic adenoviral vectors that contain a cancer selective regulatory region operatively linked to an adenoviral gene essential for adenoviral replication are known (see, e.g., U.S. Pat. No. 5,998,205). Adenoviral genes essential for replication include, but are not limited to, E1a, E1b, E2a, E2b and E4. For example, an exemplary oncolytic adenoviral vector has a cancer selective regulatory region operatively linked to the E1a gene. In other embodiments, the oncolytic adenoviral vector has a cancer selective regulatory region of the present invention operatively linked to the E1a gene and a second cancer selective regulatory region operatively linked to the E4 gene. The vectors also can include at least one therapeutic transgene, such as, but not limited to, a polynucleotide encoding a cytokine such as GM-CSF that can stimulate a systemic immune response against tumor cells.

Other exemplary oncolytic adenoviral vectors include those in which expression of an adenoviral gene, which is essential for replication, is controlled by E2F-responsive promoters, which are selectively transactivated in cancer cells. Thus, vectors that contain an adenoviral nucleic acid backbone that contain in sequential order: A left ITR, an adenoviral packaging signal, a termination signal sequence, an E2F responsive promoter which is operably linked to a first gene, such as E1a, essential for replication of the recombinant viral vector and a right ITR (see, published International PCT application No. WO02/06786, and U.S. Pat. No. 5,998,205).

In other embodiments, the oncolytic adenoviral vector has a cancer selective regulatory region operatively linked to the E1a gene and a second cancer selective regulatory region operatively linked to the E4 gene. The vectors also can carry at least one therapeutic transgene, such as, but not limited to, a polynucleotide encoding a cytokine such as GM-CSF that can stimulate a systemic immune response against tumor cells.

3. Packaging

The viral particles provided herein can be made by any method known to those of skill in the art. Generally they are prepared by growing the adenovirus vector that contains nucleic acid that encodes the modified or heterologous capsid protein in standard adenovirus packaging cells to produce particles that express the modified or heterologous capsid proteins. Alternatively, the vectors do not encode fiber proteins. Such vectors are packaged in producer cells to produce particles that express the modified fiber proteins.

As discussed, recombinant adenoviral vectors generally have at least a deletion in the first viral early gene region, referred to as E1, which includes the E1a and E1b regions. Deletion of the viral E1 region renders the recombinant adenovirus defective for replication and incapable of producing infectious viral particles in subsequently infected target cells. Thus, to enable E1-deleted adenovirus genome replication and to produce virus particles requires a system of complementation which provides the missing E1 gene product. E1 complementation is typically provided by a cell line expressing E1, such as the human embryonic kidney packaging cell line, i.e. an epithelial cell line, called 293. Cell line 293 contains the E1 region of adenovirus, which provides E1 gene region products to “support” the growth of E1-deleted virus in the cell line (see, e.g., Graham et al., J. Gen. Virol. 36: 59-71, 1977). Additionally, cell lines that may be usable for production of defective adenovirus having a portion of the adenovirus E4 region have been reported (WO 96/22378). Multiply deficient adenoviral vectors and complementing cell lines have also been described (WO 95/34671, U.S. Pat. No. 5,994,106).

For example, copending U.S. application Ser. No. 09/482,682 (also filed as International PCT application No. PCT/EP00/00265, filed Jan. 14, 200, published as International PCT application No. WO/0042208) provides packaging cell lines that support viral vectors with deletions of major portions of the viral genome, without the need for helper viruses and also provides cell lines and helper viruses for use with helper-dependent vectors. The packaging cell line has heterologous DNA stably integrated into the chromosomes of the cellular genome. The heterologous DNA sequence encodes one or more adenovirus regulatory and/or structural polypeptides that complement the genes deleted or mutated in the adenovirus vector genome to be replicated and packaged.

Packaging cell lines express, for example, one or more adenovirus structural proteins, polypeptides, or fragments thereof, such as penton base, hexon, fiber, polypeptide IIIa, polypeptide V, polypeptide VI, polypeptide VII, polypeptide VIII, and biologically active fragments thereof. The expression can be constitutive or under the control of a regulatable promoter. These cell lines are particularly designed for expression of recombinant adenoviruses intended for delivery of therapeutic products. For use herein, such packaging cell lines can express the modified or heterologous capsid proteins, such as the fiber proteins whose binding and infection of dendritic cells is enhanced.

Particular packaging cell lines complement viral vectors having a deletion or mutation of a DNA sequence encoding an adenovirus structural protein, regulatory polypeptides E1A and E1B, and/or one or more of the following regulatory proteins or polypeptides: E2A, E2B, E3, E4, L4, or fragments thereof.

The packaging cell lines are produced by introducing each DNA molecule into the cells and then into the genome via a separate complementing plasmid or plurality of DNA molecules encoding the complementing proteins can be introduced via a single complementing plasmid. Of interest herein, is a variation in which the complementing plasmid includes DNA encoding adenovirus fiber protein (or a chimeric or modified variant thereof), from Ad virus of subgroup D, such as Ad19p or Ad37.

For applications, such as therapeutic applications, the delivery plasmid further can include a nucleotide sequence encoding a heterologous polypeptide. Exemplary delivery plasmids include, but are not limited to, pDV44, pΔE1Bβ-gal and pΔE1sp1B (Microbix Biosystems; see also, U.S. Pat. No. 6,140,087 and U.S. Pat. No. 6,379,943). In a similar or analogous manner, therapeutic nucleic acids, such as nucleic acids that encode therapeutic genes, can be introduced.

The cell further includes a complementing plasmid encoding a fiber or other capsid protein as contemplated herein; the plasmid or portion thereof is integrated into a chromosome(s) of the cellular genome of the cell.

Typically, the packaging cell lines will contain nucleic acid encoding the capsid protein or modified capsid protein stably integrated into a chromosome or chromosomes in the cellular genome. The packaging cell line can be derived from a procaryotic cell line or from a eukaryotic cell line. While various embodiments suggest the use of mammalian cells, and more particularly, epithelial cell lines, a variety of other, non-epithelial cell lines are used in various embodiments. Thus, while various embodiments disclose the use of a cell line selected from among the 293, A549, W162, HeLa, Vero, 211, and 211A cell lines, any other cell lines suitable for such use are likewise contemplated herein.

4. Propagation and Scale-Up of Doubly-Ablated Adenoviral Vectors

Since doubly ablated adenoviral vectors containing mutations in the fiber and/or penton capsid proteins result in inefficient cell binding and entry via the CAR/αv integrin entry pathway, scaled up technologies improve the growth and propagation of such vectors to produce high titers of the adenoviral vectors for clinical use. Thus, also provided is a method for scaling up the production of detargeted adenoviral vectors. The detargeted adenoviral vectors comprise an adenoviral vector modified to ablate the interaction of said vector with at least one host cell receptor compared with a wild-type adenoviral vector. The detargeted adenoviral vectors can comprise an adenoviral vector modified to ablate the interaction of said vector with one, two, three or more host cell receptors. Thus, the method is suitable for producing the detargeted adenoviral vectors disclosed herein.

As noted, growth and propagation of doubly and fully ablated adenoviral vectors is enhanced by new scale up technologies. Doubly ablated vectors contain mutations in the fiber and penton capsid proteins that result in inefficient cell binding and entry via the normal cellular entry pathway using CAR and integrins. These vectors are fully detargeted in vitro and, thus, alternative cellular entry strategies allow for the efficient growth and generation of high titer preparations.

Two strategies have been envisioned to scale up vectors that are detargeted via fiber and/or penton modifications. These include: (a) the use of pseudoreceptor cell lines engineered to express a surface receptor that binds a ligand displayed on the vector (see, e.g., International PCT application No. WO 98/54346) and (b) complementing cell lines that are engineered to express native fiber and that can be engineered to express native fiber and penton (see, e.g., International PCT application No. WO 00/42208). Although these systems have shown promise for scaling up ablated adenoviral vectors, there is a need to develop a system for the simple, efficient production of the fully detargeted adenoviral vector for therapeutic uses.

Provided herein is a scale-up method for the propagation of detargeted adenoviral vectors. The method uses polycations and/or bifunctional reagents, which when added to tissue culture medium, bind adenoviral particles and direct their entry into the producer cells.

Reagents (also called medium additives) also can be included in the tissue culture medium containing producer cells to be infected with the detargeted adenoviral vectors. Alternatively the reagents can be pre-mixed with the virus, which mixture is then added to the tissue producer cells. The reagents can be added to tissue culture medium containing producer cells, or producer cells can be added to tissue culture medium containing the reagents. Any suitable producer cell known to the skilled artisan can be used in the present methods. The reagents can be added at the same time that the producer cells are infected with detargeted adenoviral vectors. Generally the reagents are present in the tissue culture medium prior to infection by the detargeted adenoviral vectors. The medium additives are maintained in the tissue culture medium during vector growth, spread and propagation. High titer yields of adenoviral vectors are obtained by this method.

Reagents which are useful in this method are those that are capable of directing adenoviral particle entry into the producer cells. Such reagents include, but are not limited to, polycations and bifunctional reagents. Suitable polycations include, but are not limited to, polytheylenimine; protamine sulfate; poly-L-lysine hydrobromide; poly(dimethyl diallyl ammonium) chloride (Merquat(r)-100, Merquat(r)280, Merquat(r)550); poly-L-arginine hydrochloride; poly-L-histidine; poly(4-vinylpyridine), poly(4-vinylpyridine) hydrochloride; poly(4-vinylpyridine)cross-linked, methylchloride quaternary salt; poly(4-vinylpyridine-co-styrene); poly(4-vinylpyridinium poly(hydrogen fluoride)); poly(4-vinylpyridinium-P-toluene sulfonate); poly(4-vinylpyridinium-tribromide); poly(4-vinylpyrrolidone-co-2-dimethylamino-ethyl methacrylate); polyvinylpyrrolidone, cross-linked; poly vinylpyrrolidone, poly(melamine-co-formaldehyde); partially methylated; hexadimethrine bromide; poly(Glu, Lys) 1:4 hydrobromide; poly(Lys, Ala) 3:1 hydrobromide; poly(Lys, Ala) 2:1 hydrobromide; poly-L-lysine succinylated; poly(Lys, Ala) 1:1 hydrobromide; and poly(Lys, Trp) 1:4 hydrobromide.

Suitable bifunctional reagents include, but are not limited to, antibodies or peptides that bind to the adenoviral capsid and that also contain a ligand that allows interaction with specific cell surface receptors of the producer cells. Examples of bifunctional reagents include: (a) anti-fiber antibody ligand fusions, (b) anti-fiber-Fab-FGF conjugate, (c) anti-penton-antibody ligand fusions, (d) anti-hexon antibody ligand fusions and (e) polylysine-peptide fusions. The ligand is any ligand that will bind to any cell surface receptor found on the producer cells.

### F. ADENOVIRUS EXPRESSION VECTOR SYSTEMS

The adenovirus vector genome that is encapsulated in the virus article and that expresses exogenous genes in a gene therapy setting is provided. The components of an recombinant adenovirus vector genome include the ability to express selected adenovirus structural genes, to express a desired exogenous protein, and to contain sufficient replication and packaging signals that the genome is packaged into a gene delivery vector particle. An exemplary replication signal is an adenovirus inverted terminal repeat containing an adenovirus origin of replication, as is well known and described herein. Although adenovirus include many proteins, not all adenovirus proteins are required for assembly of a recombinant adenovirus particle (vector). Thus, deletion of the appropriate genes from a recombinant Ad vector permits accommodation of even larger “foreign” DNA segments.

One recombinant adenovirus vector genome is “helper independent” so that genome can replicate and be packaged without the help of a second, complementing helper virus. Complementation is provided by a packaging cell. Particularly contemplated are helper dependent systems. In an exemplary embodiment, the adenovirus vector genome does not encode a functional adenovirus fiber protein. A non-functional fiber gene refers to a deletion, mutation or other modification to the adenovirus fiber gene such that the gene does not express any or insufficient adenovirus fiber protein to package a fiber-containing adenovirus particle without complementation of the fiber gene by a complementing plasmid or packaging cell line. Such a genome is referred to as a “fiberless” genome, not to be confused with a fiberless particle. Alternatively, a fiber protein may be encoded but is insufficiently expressed to result in a fiber containing particle.

Thus, contemplated for use are helper-independent fiberless recombinant adenovirus vector genomes that include genes that (a) epxress all adenovirus structural gene products but express insufficient adenovirus fiber protein to package a fiber-containing adenovirus particle without complementation of said fiber gene, (b) express an exogenous protein, and (c) contains an adenovirus packaging signal and inverted terminal repeats containing adenovirus origin of replication.

The adenovirus vector genome is propagated in vitro in the form of rDNA plasmids containing the genome, and upon introduction into an appropriate host, the viral genetic elements provide for viral genome replication and packaging rather than plasmid-based propagation. Exemplary methods for preparing an Ad-vector genome are described in the Examples.

A vector herein includes a nucleic acid (such as DNA) molecule capable of autonomous replication in a cell and to which a DNA segment, e.g., a gene or polynucleotide, can be operatively linked to bring about replication of the attached segment. For purposes herein, one of the nucleotide segments to be operatively linked to vector sequences encodes at least a portion of a therapeutic nucleic acid molecule. As noted above, therapeutic nucleic acid molecules include those encoding proteins and also those that encode regulatory factors that can lead to expression or inhibition or alteration of expression of a gene product in a dendritic cell.

1. Nucleic Acid Gene Expression Cassettes

In various embodiments, a peptide-coding sequence of the therapeutic gene is inserted into an expression vector and expressed; however, it also is feasible to construct an expression vector which also includes some non-coding sequences as well. Generally, however, non-coding sequences are excluded. Alternatively, a nucleotide sequence for a soluble form of a polypeptide may be utilized. Another therapeutic viral vector includes a nucleotide sequence encoding at least a portion of a therapeutic nucleotide sequence operatively linked to the expression vector for expression of the coding sequence in the therapeutic nucleotide sequence.

The choice of viral vector into which a therapeutic nucleic acid molecule is operatively linked depends directly, as is well known in the art, on the functional properties desired, e.g., vector replication and protein expression, and the host cell to be transformed—these being limitations inherent in the art of constructing recombinant DNA molecules. Although certain adenovirus serotypes are recited herein in the form of specific examples, it should be understood that the use of any adenovirus serotype, including hybrids and derivatives thereof are contemplated. Of particular interest, is the use of fiber that targets the resulting viral particle to dendritic cells.

2. Promoters

As noted elsewhere herein, an expression nucleic acid in an Ad-derived vector also include a promoter, particularly a tissue or cell specific promoter, such as one expressed dendritic cells. Promoters are nucleic acid fragments that contain a DNA sequence that controls the expression of a gene located 3′ or downstream of the promoter. The promoter is the DNA sequence to which RNA polymerase specifically binds and initiates RNA synthesis (transcription) of that gene, typically located 3′ of the promoter. A promoter also includes DNA sequences which direct the initiation of transcription, including those to which RNA polymerase specifically binds. If more than one nucleic acid sequence encoding a particular polypeptide or protein is included in a therapeutic viral vector or nucleotide sequence, more than one promoter or enhancer element may be included, particularly if that would enhance efficiency of expression. Regulatable (inducible) as well as constitutive promoters may be used, either on separate vectors or on the same vector. For example, some useful regulatable promoters are those of the CREB-regulated gene family and include inhibin, gonadotropin, cytochrome c, glucagon and other. (See, e.g., International PCT application No. WO 96/14061). The promoter selected can be selected from a dendritic cell-specific gene, such as NFκB.

A regulatable or inducible promoter is a promoter where the rate of RNA polymerase binding and initiation is modulated by external stimuli. (see, e.g., U.S. Pat. Nos. 5,750,396 and 5,998,205). Such stimuli include various compounds or compositions, light, heat, stress, chemical energy sources, and the like. Inducible, suppressible and repressible promoters are considered regulatable promoters. Regulatable promoters also can include tissue-specific promoters. Tissue-specific promoters direct the expression of the gene to which they are operably linked to a specific cell type. Tissue-specific promoters cause the gene located 3′ of it to be expressed predominantly, if not exclusively, in the specific cells where the promoter expressed its endogenous gene. Typically, it appears that if a tissue-specific promoter expresses the gene located 3′ of it at all, then it is expressed appropriately in the correct cell types (see, e.g., Palmiter et al. (1986) Ann. Rev. Genet. 20: 465-499).

### G. HETEROLOGOUS POLYNUCLEOTIDES AND THERAPEUTIC NUCLEIC ACIDS

The packaged adenoviral genome also can contain a heterologous polynucleotide that encodes a product of interest, such as a therapeutic protein. Adenoviral genomes containing heterologous polynucleotides are well known (see, e.g., U.S. Pat. Nos. 5,998,205, 6,156,497, 5,935,935, and 5,801,029). These can be used for in vitro and in vivo delivery of the products of heterologous polynucleotides or the heterologous polynucleotides.

The adenoviral particles provided herein can be used to engineer a cell to express a protein that it otherwise does not express or does not express in sufficient quantities. This genetic engineering is accomplished by infecting the desired cell with an adenoviral particle whose genome includes a desired heterologous polynucleotide. The heterologous polynucleotide is then expressed in the genetically engineered cells. For use herein the cell is generally a mammalian cell, and is typically a primate cell, including a human cell. The cell can be inside the body of the animal (in vivo) or outside the body (in vitro). Heterologous polynucleotides (also referred to as heterologous nucleic acid sequences) are included in the adenoviral genome within the particle and are added to that genome by techniques known in the art. Any heterologous polynucleotide of interest can be added, such as those disclosed in U.S. Pat. No. 5,998,205, incorporated herein by reference.

Polynucleotides that are introduced into an Ad genome or vector can be any that encode a protein of interest or that are regulatory sequences. In particular, the genomes can include heterologous nucleic acid encoding a product for expression in a dendritic cell for presentation or to alter the activity of the dendritic cell. For purposes herein, proteins include, but are not limited to tumor antigens. Tumor antigens included, but are not limited to carcinoembryonic antigen, NY-BR1, NY-ESO-1, MAGE-1, MAGE-3, BAGE, GAGE, SCP-1, SSX-1, SSX-2, SSX-4, CT-7, Her2/Neu, NY-BR-62, NY-BR-85 and tumor protein D52 (Scanlan and Jäger (2001) Breast Cancer Res. 3:95-98; Yu and Restifo (2002) J. Clin. Invest. 110:289-94). The following Table includes an exemplary list of tumor antigens and tissues expressing such antigens.

| Antigen | Tissue |
|---|---|
| Oncofetal | |
| OPA | Fetal pancreas |
| CEA | Colon, Rectal, Stomach, Lung, Pancreas, |
| | Kidney, Bladder, Head & Neck, Cervical, |
| | endometrial, ovarian, Breast |
| POA | Fetal pancreas |
| FAP | Fetal pancreas |
| PA8-15 | Pancreatic cancer |
| | cell line SUIT-2 |
| Adult | |
| CA 50 | Colorectal |
| | carcinoma cell |
| | line |
| CA 19-9 | Colon carcinoma |
| | cell line SW1116 |
| CA 242 | Colorectal |
| | carcinoma cell |
| | line COLO 205 |
| CAR-3 | Epidermoid |
| | carcinoma cell |
| | line A 431 |
| DU-PAN-2 | Pancreatic |
| | carcinoma cell |
| | line HPAF |
| Ypan-1 | Pancreatic |
| | carcinoma cell |
| | line SW1990 |
| Span-1 | Pancreatic |
| | carcinoma cell |
| | line SW1990 |
| BW494 | Pancreatic tumor |
| | tissue |
| MUSE 11 | Gastric cancer |
| | ascites fluid |
| L_{A1} | Embryonal carcinoma cells |
| Le^{a} | Colon adenocarcinoma |
| Fuc-L_{A1} | Pancreatic adenocarcinoma |
| Le^{b} | Colon |
| | adenocarcinoma |
| | Pancreatic |
| | adenocarcinoma |
| 3-isoL_{M1} | Small cell lung |
| | carcinoma |
| | Glioma |
| | Medulloblastoma |
| | Teratocarcinoma |
| | cells |
| 3′,6′-isoL_{D1} | Liver metastasis |
| | of colon cancer |
| | Embryonal |
| | carcinoma cells |
| Fuc-3′- | Gastrointestinal |
| isoL_{M1} | cancer |
| Sialylated | |
| Le^{a} | |
| Fuc-3′,6′- | Human colon |
| isoL_{D1} | adenocarcinoma |
| Disialylated | |
| Le^{a} | |
| nL_{A1} | Colon cancer |
| i-Antigen | Lung cancer |
| SSEA-1 | Teratocarcinoma |
| Le^{x} | Colon cancer |
| Fuc-nL_{A1} | |
| Dimeric Le^{x} | Adenocarcinoma |
| | Colon cancer |
| | Liver cancer |
| Le^{v} | Gastric cancer |
| | Breast cancer |
| | Colon cancer |
| 6′-L_{M1} | Colorectal |
| | carcinoma |
| | Lung carcinomas |
| | Primary |
| | hepatoma |
| Sialylated | Gastrointestinal |
| Le^{x} | cancer |
| or | Lung carcinoma |
| Fuc-3′-L_{M1} | |
| | Gastric |
| | colon |
| | lung |
| | breast |
| | renal cancers |
| GB3 | Burkitt's lymphoma |
| Globo-H | breast cancer |
| Sulfatide | Mucinous cystadenocarcinoma, |
| Disulfated G_{A1} | Hepatocellular carcinoma |
| N-Glycolyl- | Colon cancer |
| neuraminic acid | |
| N-Glycolyl-G_{M2} | N-Glycolyl-G_{M2} |
| G_{M2} | Melanoma |
| OFA-I-1 | |
| OFA-I-2 | |
| | Glioma |
| | Germ cell tumors |
| G_{D2} | Melanoma |
| | Neuroblastoma |
| | Small cell lung carninoma |
| | Glioma |
| G_{M3} | Melanoma |
| Ag | |
| FCM1 | |
| 2-39 | |
| IF43 | |
| gp-100 melanoma- | |
| associated | |
| antigen | |
| G_{D3} | Melanoma |
| HJM1 | Melanoma |
| | Medulloblastoma |
| | Glioma |
| | Leukemia |
| | Meninglioma |
| 9-O-Acetyl-G_{D3} | Melanoma |
| Fuc-G_{M1} | Small cell lung carcinoma |
| COTA | Colon, ovarian |
| SW1038 | Colon |
| CTS | prostate |
| MAGE-1 | Lung |
| MAGE-2 | melanocyte |
| MAGE-3 | breast |
| (MZ2-E | |
| MZ2-Bb) | |
| MUC-1 | Breast |
| | pancreas |
| Lewis-Ag (GICA) | Ovarian |
| | myelin |
| TAG-12 | Breast |
| | ovarian |
| TAG-72 | colon |
| | ovarian pancrease |
| Orfan-specific | Lung |
| cancer neoantigen | |
| (OSN) | |
| GP100 | Melanocyte |
| MART-1 | Melanocyte |
| p95/p97 | Melanocyte |
| EGF receptor | Squamous tumors |
| CA125 | Ovary |
| | Breast |
| p97 | Melanocyte |
| (melanotrans- | |
| ferrin) | |
| 22-1-1 | uterus |
| | cervix |
| | ovary |
| GA733 | gastrointestinal |
| | carcinoma |
| YH206 | adenocarcinomas |
| MART-2 | melanocytes |
| BAGE-1 | melanocytes |
| GAGE1-6 | melaocyte |
| | osteocarcoma |
| DF3 | Breast |
| | lymphocytes |
| L3p40-50 | Lung |
| L3p90 | |
| Thomsen- | pancarcinoma |
| Friedenrich | |
| Pan Tumor | |
| Antigen | |
| | pancreas |
| | ovarian |
| EPB-2 | B cell lymphoma |
| | melanoma |
| | lymphoma |
| | medullary thyroid |
| | carcinoma |
| | gastrointestinal |
| | carcinoma |
| NS-ESO-1 | melanoma, breast, bladder, prostate, |
| | heptocellular carcinoma |
| NY-ESO-1 | melanoma, breast, bladder, prostate, |
| | heptocellular carcinoma |

Proteins also include, but are not limited to, therapeutic proteins, such as an immunostimulating protein, such as an interleukin, interferon, or colony stimulating factor, such as granulocyte macrophage colony stimulating factor (GM-CSF; see, e.g., 5,908,763F. Generally, such GM-CSF is a primate GM-CSF, including human GM-CSF. Other immuno-stimulatory genes include, but are not limited to, genes that encode cytokines IL-1, IL-2, IL-4, IL-5, IFN, TNF, IL-12, IL-18, and flt3, proteins that stimulate interactions with immune cells (B7, CD28, MHC class I, MHC class II, TAPs), tumor-associated antigens (immunogenic sequences from MART-1, gp100 (pmel-17), tyrosinase, tyrosinase-related protein 1, tyrosinase-related protein 2, melanocyte-stimulating hormone receptor, MAGE1, MAGE2, MAGE3, MAGE12, BAGE, GAGE, NY-ESO-1, -catenin, MUM-1, CDK-4, caspase 8, KIA 0205, HLA-A2R1701, -fetoprotein, telomerase catalytic protein, G-250, MUC-1, carcinoembryonic protein, p53, Her2/neu, triosephosphate isomerase, CDC-27, LDLR-FUT, telomerase reverse transcriptase, and PSMA), cDNAs of antibodies that block inhibitory signals (CTLA4 blockade), chemokines (MIP1, MIP3, CCR7 ligand, and calreticulin), and other proteins.

Other polynucleotides, including therapeutic nucleic acids, such as therapeutic genes, of interest include, but are not limited to, anti-angiogenic, and suicide genes. Anti-angiogenic genes include, but are not limited to, genes that encode METH-1, METH-2, TrpRS fragments, proliferin-related protein, prolactin fragment, PEDF, vasostatin, various fragments of extracellular matrix proteins and growth factor/cytokine inhibitors. Various fragments of extracellular matrix proteins include, but are not limited to, angiostatin, endostatin, kininostatin, fibrinogen-E fragment, thrombospondin, tumstatin, canstatin, and restin. Growth factor/cytokine inhibitors include, but are not limited to, VEGF/VEGFR antagonist, sFlt-1, sFlk, sNRP1, angiopoietin/tie antagonist, sTie-2, chemokines (IP-10, PF-4, Gro-beta, IFN-gamma (Mig), IFN, FGF/FGFR antagonist (sFGFR), Ephrin/Eph antagonist (sEphB4 and sephrinB2), PDGF, TGF and IGF-1.

A “suicide gene” encodes a protein that can lead to cell death, as with expression of diphtheria toxin A, or the expression of the protein can render cells selectively sensitive to certain drugs, e.g., expression of the Herpes simplex thymidine kinase gene (HSV-TK) renders cells sensitive to antiviral compounds, such as acyclovir, gancyclovir and FIAU (1-(2-deoxy-2-fluoro-β-D-arabinofuranosil)-5-iodouracil). Other suicide genes include, but are not limited to, genes that encode carboxypeptidase G2 (CPG2), carboxylesterase (CA), cytosine deaminase (CD), cytochrome P450 (cyt-450), deoxycytidine kinase (dCK), nitroreductase (NR), purine nucleoside phosphorylase (PNP), thymidine phosphorylase (TP), varicella zoster virus thymidine kinase (VZV-TK), and xanthine-guanine phosphoribosyl transferase (XGPRT). Alternatively, a therapeutic nucleic acid can exert its effect at the level of RNA, for instance, by encoding an antisense message or ribozyme, a protein that affects splicing or 3′ processing (e.g., polyadenylation), or a protein that affects the level of expression of another gene within the cell, e.g. by mediating an altered rate of mRNA accumulation, an alteration of mRNA transport, and/or a change in post-transcriptional regulation. The addition of a therapeutic nucleic acid to a virus results in a virus with an additional antitumor mechanism of action. Thus, a single entity (i.e., the virus carrying a therapeutic transgene) is capable of inducing multiple antitumor mechanisms. Other encoded proteins, include, but are not limited to, herpes simplex virus thymidine kinase (HSV-TK), which is useful as a safety switch (see, U.S. patent application Ser. No. 08/974,391, filed Nov. 19, 1997, which published as PCT Publication No. WO/9925860), Nos, FasL, and sFasR (soluble Fas receptor).

Also contemplated are combinations of two or more transgenes with synergistic, complementary and/or nonoverlapping toxicities and methods of action. The resulting adenovirus can retain the viral oncolytic functions and, for example, additionally are endowed with the ability to induce immune and anti-angiogenic responses and other responses as desired.

Therapeutic polynucleotides and heterologous polynucleotides also include those that exert an effect at the level of RNA or protein. These include a factor capable of initiating apoptosis, RNA, such as RNAi and other double-stranded RNA, antisense and ribozymes, which among other capabilities can be directed to mRNAs encoding proteins essential for proliferation, such as structural proteins, transcription factors, polymerases, genes encoding cytotoxic proteins, genes that encode an engineered cytoplasmic variant of a nuclease (e.g. RNase A) or protease (e.g. trypsin, papain, proteinase K and carboxypeptidase). Other polynucleotides include a cell or tissue specific promoters, such as those used in oncolytic adenoviruses (see, e.g., U.S. Pat. No. 5,998,205).

The heterologous polynucleotide encoding a polypeptide also can contain a promoter operably linked to the coding region. Generally the promoter is a regulated promoter and transcription factor expression system, such as the published tetracycline-regulated systems, or other regulatable systems (WO 01/30843), to allow regulated expression of the encoded polypeptide. Exemplary of other promoters, are tissue-selective promoters, such as those described in U.S. Pat. No. 5,998,205. An exemplary regulatable promoter system is the Tet-On (and Tet-Off) system currently available from Clontech (Palo Alto, Calif.). This promoter system allows the regulated expression of the transgene controlled by tetracycline or tetracycline derivatives, such as doxycycline. This system can be used to control the expression of the encoded polypeptide in the viral particles and nucleic acids provided herein. Other regulatable promoter systems are known (see, e.g., published U.S. Application No. 20020168714, entitled “Regulation of Gene Expression Using Single-Chain, Monomeric, Ligand Dependent Polypeptide Switches,” which describes gene switches that contain ligand binding domains and transcriptional regulating domains, such as those from hormone receptors). Other suitable promoters that can be employed include, but are not limited to, adenoviral promoters, such as the adenoviral major late promoter and/or the E3 promoter; or heterologous promoters, such as the cytomegalovirus (CMV) promoter; the Rous Sarcoma Virus (RSV) promoter; inducible promoters, such as the MMT promoter, the metallothionein promoter; heat shock promoters; the albumin promoter; and the ApoAl promoter.

Therapeutic transgenes can be included in the viral constructs and resulting particles. Among these are those that result in an “armed” virus. For example, rather than delete E3 region as in some embodiments described herein, all or a part of the E3 region can be preserved or re-inserted in an oncolytic adenoviral vector (discussed above). The presence of all or a part of the E3 region can decrease the immunogenicity of the adenoviral vector. It also increases cytopathic effect in tumor cells and decreases toxicity to normal cells. Typically such vector expresses more than half of the E3 proteins.

Adenoviruses for therapy, including those for human therapy, are known. Such known viruses can be modified as provided herein to increase infection of dendritic cells and/or increasing binding to receptors expressed on dendritic cells. The adenoviral vectors that are used to produce the viral particles can include other modifications. Modifications include modifications to the adenovirus genome that is packaged in the particle in order to make an adenoviral vector. As discussed above, adenovirus vectors and particles with a variety of modifications are available. Modifications to adenoviral vectors include deletions known in the art, such as deletions in one or more of the E1, E2a, E2b, E3, or E4 coding regions. These adenoviruses are sometimes referred to as early generation adenoviruses and include those with deletions of all of the coding regions of the adenoviral genome (“gutless” adenoviruses, discussed above) and also include replication-conditional adenoviruses, which are viruses that replicate in certain types of cells or tissues but not in other types as a result of placing adenoviral genes essential for replication under control of a heterologous promoter (discussed above; see also U.S. Pat. No. 5,998,205, U.S. Pat. No. 5,801,029; U.S. patent application 60/348,670 and corresponding published International PCT application No. WO 02/06786). These include the cytolytic, cytopathic viruses (or vectors), including the oncolytic viruses discussed above.

Alternatively, as discussed above, the vector can include a mutation or deletion in the E1b gene. Typically such mutation or deletion in the E1b gene is such that the E1b-19 kD protein becomes non-functional. This modification of the E1b region can be combined with vectors where all or a part of the E3 region is present.

### H. FORMULATION AND ADMINISTRATION

1. Formulation

Compositions containing therapeutically effective for concentrations of recombinant adenovirus delivery vectors for delivery of therapeutic gene products to target cells and/or tissues (i.e. dendritic cells). Modes of administration include, but are not limited to, intramuscular, parenteral, local, topical and other routes whereby dendritic cells can be targeted.

The recombinant viral compositions also can be formulated for in sustained released formulations, such as adsorbed to biodegradable supports, including collagen sponges, or in liposomes. Sustained release formulations can be formulated for multiple dosage administration, so that during a selected period of time, such as a month or up to about a year, several dosages are administered. Thus, for example, liposomes can be prepared such that a total of about two to up to about five or more times the single dosage is administered in one injection. The vectors are formulated in pharmaceutically acceptable carriers.

The composition can be provided in a sealed sterile vial containing an amount such that upon administration a sufficient amount of viral particles is delivered where about 50 to 150 μl, containing at least about 10⁷, or 10⁸ plaque forming units (pfu) in such volume are delivered and at least 10⁹-10¹⁰ pfu are delivered.

To prepare compositions the viral particles are dialyzed into a suitable carrier or viral particles can be concentration and/or mixed therewith. The resulting mixture can be a solution, suspension or emulsion. In addition, the viral particles may be formulated as the sole pharmaceutically active ingredient in the composition or may be combined with other active agents for the particular disorder treated.

Exemplary suitable carriers include, but are not limited to, physiological saline, phosphate buffered saline (PBS), balanced salt solution (BSS), lactate Ringers solution, and solutions containing thickening and solubilizing agents, such as glucose, polyethylene glycol, and polypropylene glycol and mixtures thereof. Liposomal suspensions also can be suitable as pharmaceutically acceptable carriers. These can be prepared according to methods known to those skilled in the art.

The compositions can be prepared with carriers that protect them against rapid elimination from the body, such as time release formulations or coatings. Such carriers include controlled release formulations, such as, but not limited to, microencapsulated delivery systems, and biodegradable, biocompatible polymers, such as ethylene vinyl acetate, polyanhydrides, polyglycolic acid, polyorthoesters, polylactic acid and other types of implants that may be placed directly into the body. The compositions also can be administered in pellets, such as Elvax pellets (ethylene-vinyl acetate copolymer resin).

Liposomal suspensions, including tissue-targeted liposomes, also can be suitable as pharmaceutically acceptable carriers. For example, liposome formulations may be prepared by methods known to those of skill in the art (see, e.g., Kimm et al. (1983) Bioch. Bioph. Acta 728:339-398; Assil et al. (1987) Arch Opthalmol. 105:400; and U.S. Pat. No. 4,522,811). The viral particles can be encapsulated into the aqueous phase of liposome systems.

The active materials also can be mixed with other active materials, that do not impair the desired action, or with materials that supplement the desired action or have other action, including viscoelastic materials, such as hyaluronic acid, which is sold under the trademark HEALON, which is a solution of a high molecular weight (MW) of about 3 millions fraction of sodium hyaluronate (manufactured by Pharmacia, Inc; see, e.g., U.S. Pat. Nos. 5,292,362, 5,282,851, 5,273,056, 5,229,127, 4,517,295 and 4,328,803). Additional active agents may be included.

The compositions can be enclosed in ampules, disposable syringes or multiple or single dose vials made of glass, plastic or other suitable material. Such enclosed compositions can be provided in kits. In particular, kits containing vials, ampules or other container.

Finally, the vectors can be packaged as articles of manufacture containing packaging material, typically a vial, a pharmaceutically acceptable composition containing the viral particles and a label that indicates the therapeutic use of the composition.

Also provided are kits for practice of the methods herein. The kits contain one or more containers, such as sealed vials, with sufficient composition for single dosage administration, other reagents as needed, and optionally instructions for use.

Administration of the composition is typically by intravenous or intramuscular injection, although other modes of administration can be effective.

2. Administration

The compositions containing the compounds are generally administered systemically. It is further understood that, for any particular subject, specific dosage regimens should be adjusted over time according to the individual need and the professional judgment of the person administering or supervising the administration of the recombinant viruses, and that the concentration ranges set forth herein are exemplary only and are not intended to limit the scope or practice of the methods, uses, and products provided herein.

In addition to in vivo administration, the viral particles provided herein can be used in methods of ex vivo therapy in which mixtures of cells, such as bone marrow cells, that include or contain dendritic cells or that are enriched for dendritic cells are contacted with the viral particles so that dendritic cells are preferentially infected. The resulting cells are optionally culture in vitro and are then infused into a recipient subject, generally the donor.

### I. DISEASES, DISORDERS AND THERAPEUTIC PRODUCTS

Dendritic cells modified with adenoviral particles provided herein express heterologous proteins that can be presented or that can alter dendritic cell functioning. As noted, the adenoviral particles can be administered to a subject or can be contacted ex vivo with dendritic cells obtained from a donor and can be infused into a subject patient, typically the donor. The viral particles, which express fibers targeted to dendritic cells will preferentially infect dendritic cells. Dendritic cells modified to express particular antigens act as vaccines by stimulating an immune response against the presented antigen. These cells can be used for treatment or prophylaxis of virtually any bacterial, protozoan, parasitic, fungal or other infection. In addition, presentation of a tumor antigen renders such cells effective for treatment or prophylaxis of cancers.

Expression of a product that interferes with dendritic cell function, such as by blocking expression of genes, including genes encoding NFκB or RelB, prevent the dendritic cells from stimulating T-cells. Such particles and the resulting cells can be used to treat diseases such as asthma, allergies, autoimmune diseases, such as juvenile diabetes, rheumatoid arthritis, lupus and inflammatory diseases.

Pathogens include, but are not limited to, bacterial, such as E. coli and anthrax, viruses, such as vaccinia virus (i.e. small pox, chicken pox), herpes viruses, cytomegalovirus (CMV) vectors, papillomavirus, parasites and fungi. Selected antigens can be determined empirically by identifying those that are effective in generating an immunoprotective response in a model system such as a rodent model.

Treatment with the particles, either in vivo or ex vivo can be prophylactic where administration (vaccination) generates immunity or it can be for treatment of the disease.

### J. EXAMPLES

The following examples are included for illustrative purposes only and are not intended to limit the scope of the invention.

### Example 1

### Construction of Ad5 Vectors Containing the Fiber AB Loop, KO1 and Penton, PD1 Mutations and Derivatives Thereof

Three recombinant adenoviral vectors were prepared that contain the KO1 fiber or PD1 penton base mutations either alone or in combination, these vectors are designated Av3nBgFKO1 Av1nBgPD1, and Av1nBgFKO1PD1. Construction of these vectors is described below and a general description of each vector is set forth in Table 1.

TABLE 1
Description Of Detargeted Recombinant Adenoviral
Vectors Used For Scale-up Vector

| Vector | Description |
|---|---|
| Av3nBg | An E1, E2a, E3-deleted adenoviral |
| | vector encoding a nuclear localizing |
| | β-galactosidase |
| Av1nBg | An E1 and E3-deleted adenoviral |
| | vector encoding a nuclear localizing |
| | β-galactosidase |
| Av3nBgFKO1 | The same as Av3nBg but containing |
| | the KO1 mutation in the fiber gene |
| Av1nBgPD1 | The same as Av1nBg but containing |
| | the PD1 mutation in the penton gene |
| Av1nBgFKO1PD1 | The same as Av1nBg but containing |
| | the fiber KO1 and penton PD1 |
| | mutations |

### Av1nBg

This is a well-known vector, its sequence is set forth in SEQ ID NO. 51.

### Av3nBg

This is a well-known vector, its sequence is set forth in SEQ ID NO. 52.

### Av3nBgFKO1

Genetic Incorporation of the KO1 Fiber Mutation to Generate Av3nBgFKO1

The adenoviral vector Av3nBgFKO1 was generated in an E1-, E2a-, E3-deleted backbone based on the adenovirus serotype 5 genome. It contains a RSV promoted nuclear-localizing β-galactosidase gene in place of the E1 region. In addition, the fiber gene carries the KO1 mutation. This mutation results in a substitution of fiber amino acids 408 and 409, changing them from serine and proline to glutamic acid and alanine, respectively.

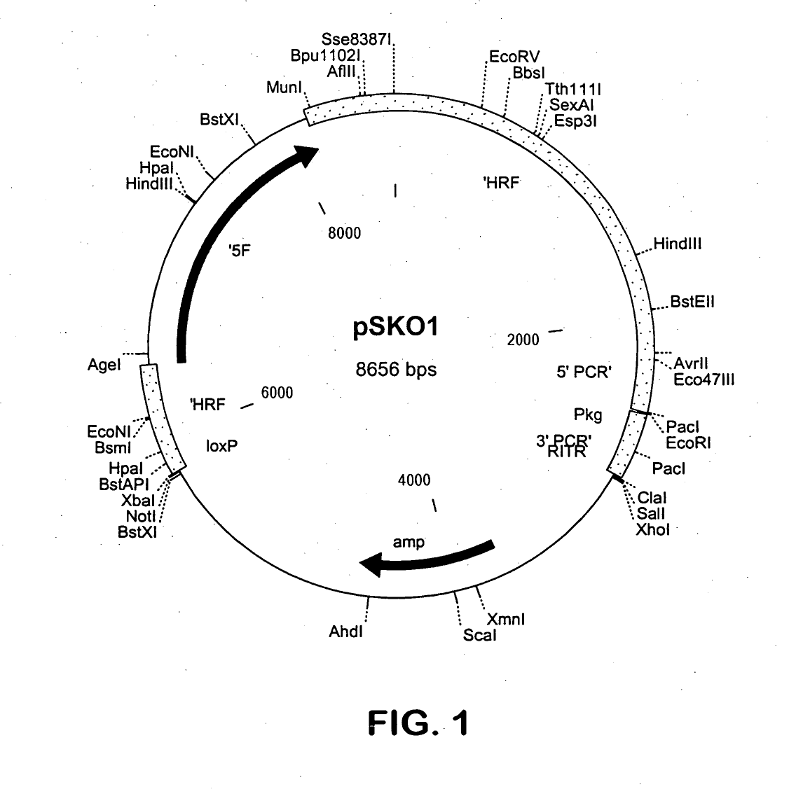

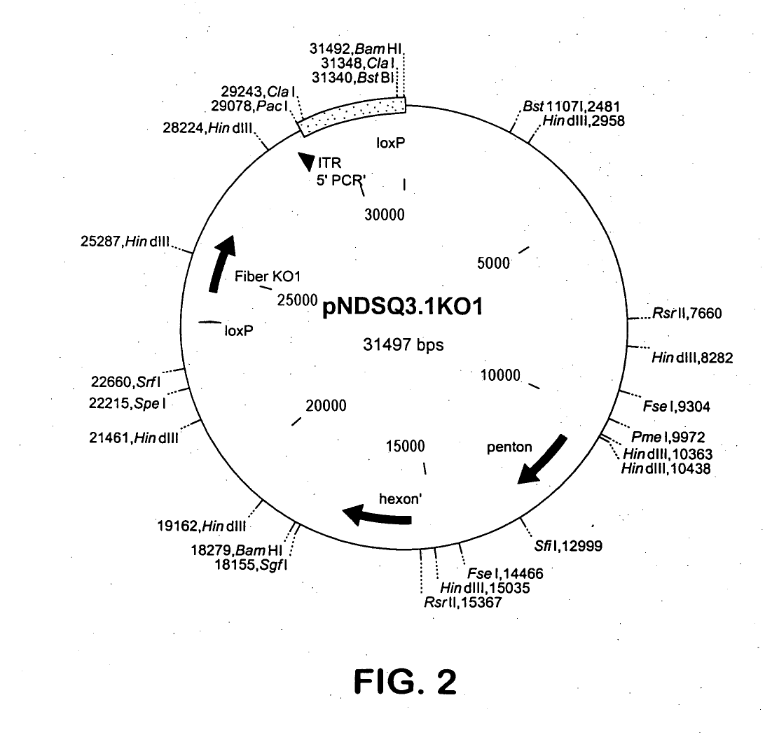

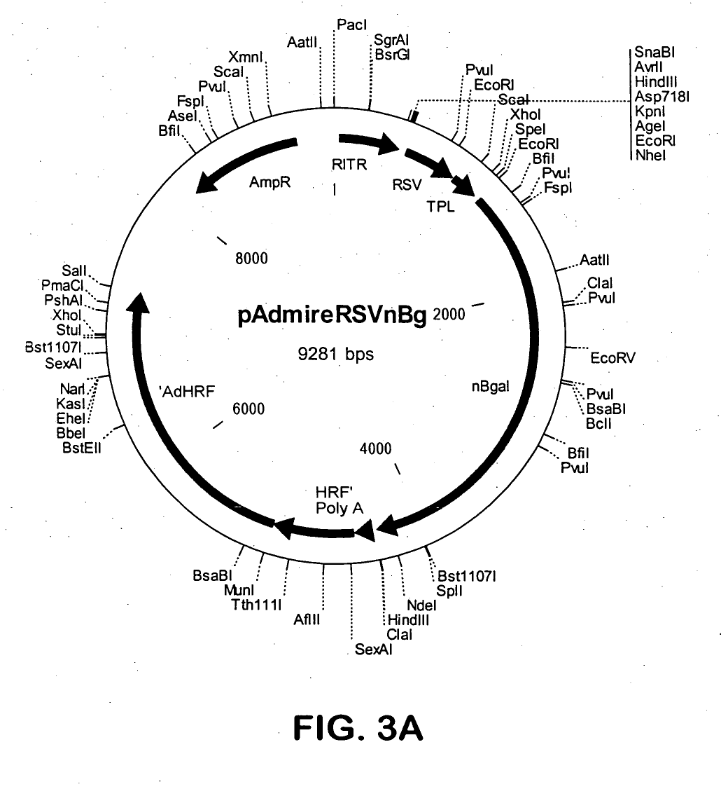

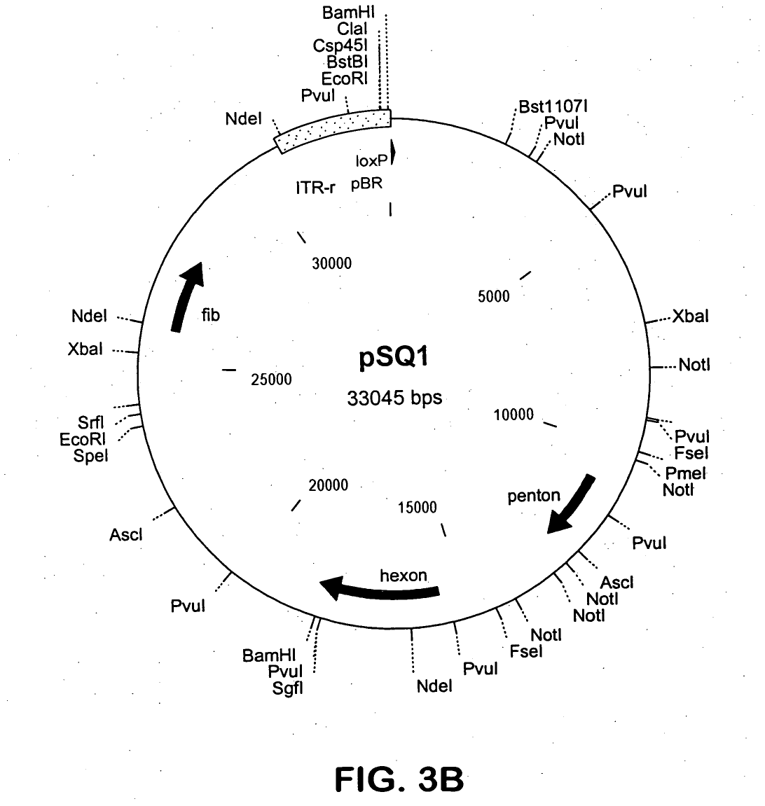

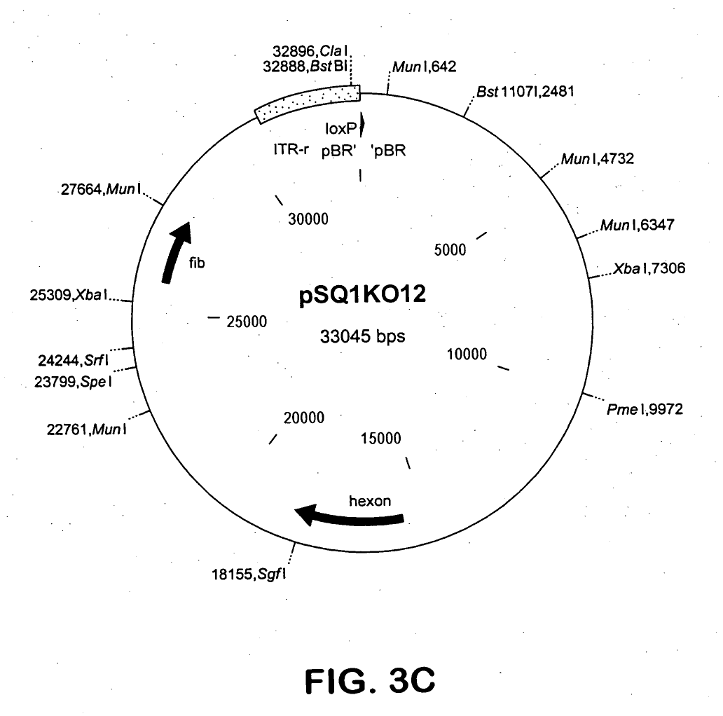

The vector was constructed as follows. First, the plasmid pSKO1 (FIG. 1) was digested with the restriction enzymes SphI and MunI. The resulting DNA fragments were separated by electrophoresis on an agarose gel. The 1601 bp fragment containing all but the 5′ end of the fiber gene was excised from the agarose gel and the DNA was isolated and purified. The fragment was then ligated with the 9236 bp fragment of p5FloxHRFRGD, which had been digested with SphI and MunI. The resulting plasmid, p5FloxHRFKO1, was digested with SpeI and PacI and the 6867 bp fragment containing the fiber gene was isolated. The fragment was ligated with the 24,630 bp SpeI-PacI fragment of pNDSQ3.1. The resulting plasmid, pNDSQ3.1KO1 (FIG. 2), was used together with pAdmireRSVnBg (FIG. 3A) to generate a plasmid which encodes the full-length adenoviral vector genome. It, however, was necessary to remove the PacI site from pNDSQ3.1KO1 (FIG. 2) prior to recombination with pAdmireRSVnBg (FIG. 3A) so that the final plasmid contains a unique PacI site adjacent to the 5′ ITR. The PacI site in pNDSQ3.1KO1 was removed by digestion with PacI followed by blunting with T4 DNA Polymerase and religation. The resulting plasmid was called pNDSQ3.1KO1 (Pac.

To generate a full-length plasmid containing the entire adenoviral genome, pAdmireRSVnBg (FIG. 3A) was digested with SalI and co-transfected into competent cells of the E. coli strain BJ5183 along with pNDSQ3.1KO1ΔPac, which had been digested with BstBI. Homologous recombination between the two plasmids generated a full-length plasmid encoding the entire adenoviral vector genome, which was called pFLAv3nBgFKO1.

The plasmid pFLAv3nBgKO1 was linearized with PacI and transfected into 633 cells. In the fiber complementing 633 cell line, the resulting viral DNA containing the KO1 mutation is capable of being packaged into infectious viral particles containing a mixture of wildtype fiber and mutant fiber proteins. After five rounds of amplification in 633 cells, a cytopathic effect was observed. Three more rounds of amplification in 633 cells were performed followed by purification of the virus by standard CsCl centrifugation procedures. This viral preparation was used to infect AE1-2a cells, which do not express fiber. The resulting virus contained only the mutant fiber protein on its capsid. Virus particles were purified by standard CsCl centrifugation procedures.

### Av1nBgFKO1

The vector Av1nBgFKO1 is made in a similar manner to Av3nBgFKO1 described above.

### Av1nBgKO12

An additional fiber AB loop mutation (described by Einfeld et al. (2001) J. Virology 75:11284-11291) was incorporated into the genome of Av1nBg. This AB loop mutation is a four amino acid substitution, R512S, A515G, E516G, and K517G, and is referred to as KO12. The KO12 mutation was incorporated into the fiber gene by PCR gene overlap extension using the plasmid pSQ1 (FIG. 3B) as template. The pSQ1 plasmid contains most of the Ad5 genome, extending from base pair 3329 through the right ITR, in a pBR322 backbone. First, a segment of the Ad5 genome extending from within the E3 region into the fiber gene was amplified by PCR using the plasmid pSQ1 as a template with the following primers termed 5FF, 5′-GAA CAG GAG GTG AGC TTA GA-3′ SEQ ID NO. 53), and 5FR, 5′-TCC GCC TCC ATT TAG TGA ACA GTT AGG AGA TGG AGC TGG TGT G-3′ (SEQ ID NO. 54). The primer 5FR contains an 18 base 5′-extension that encodes the modified fiber AB loop amino acids from 512 through 517. A second PCR using pSQ1 as a template amplified the region immediately 3′ of the AB loop substitution and extending past the MunI site located 40 base pairs 3′ of the fiber gene stop codon. The two primers used for this reaction were 3FF: 5′-TCA CTA AAT GGA GGC GGA GAT GCT AAA CTC ACT TTG GTC TTA AC-3′ (SEQ ID NO. 55), and 3FR: 5′-GTG GCA GGT TGA ATA CTA GG-3′ (SEQ ID NO. 56). The primer 3FR contains an 18 base 5′-extension that encodes the modified fiber AB loop amino acids 512 through 517. Amplified products of the expected size were obtained and used in a second PCR with the end primers 5FF and 3FR to join the fragments together. The KO12 PCR fragment was digested with XbaI and MunI cloned directly into the fiber shuttle plasmid, pFBshuttle(EcoRI) to generate the plasmid pFBSEKO12 which contains the 8.8 kB EcoRI fragment of pSQ1. The pFBSEKO12 plasmid was digested with XbaI and EcoRI and cloned into pSQ1 using a three-way ligation to generate pSQ1KO12 (FIG. 3C). The KO12 cDNA was incorporated into the genome of Av1nBg, an adenovirus vector with E1 and E3 deleted encoding β-galactosidase, by homologous recombination between ClaI-linearized pSQ1KO12 and pAdmireRSVnBg digested with SalI and PacI to generate Av1nBgKO12. The KO12 vector was transfected in 633 cells, scaled-up on non-fiber expressing cells and purified, as described above for KO1.

### Av1nBgPD1

Genetic Incorporation of the PD1 Penton Mutation to Generate Av1nBgPD1

The adenoviral vector Av1nBgPD1 is an E1-, E3-deleted vector based on the adenovirus serotype 5 genome. It contains a RSV promoted nuclear-localizing β-galactosidase gene in the E1 region and also contains the PD1 mutation in the penton gene. The PD1 mutation results in a substitution of amino acids 337 through 344 of the penton protein, HAIRGDTF (SEQ ID NO. 49), with amino acids SRGYPYDVPDYAGTS (SEQ ID NO. 50), thus replacing the RGD tripeptide (see, Einfeld et al. (2001) J. Virology 75:11284-11291). The mutation in the penton gene was generated in the plasmid pGEMpen5, which contains the Adenovirus serotype 5 penton gene. To generate the mutation, four oligonucleotides were synthesized. The sequences of the oligonucleotides were as follows: penton 1: 5′ CGC GGA AGA GAA CTC CAA CGC GGC AGC CGC GGC AAT GCA GCC GGT GGA GGA CAT GAA 3′ (SEQ ID NO. 57); penton 2: 5′ TAT CGT TCA TGT CCT CCA CCG GCT GCA TTG CCG CGG CTG CCG CGT TGG AGT TCT CTT CC 3′ (SEQ ID NO. 58); penton 3: 5′ CGA TAG CCG CGG CTA CCC CTA CGA CGT GCC CGA CTA CGC GGG CAC CAG CGC CAC ACG GGC TGA GGA GAA GCG CGC 3′ (SEQ ID NO. 59); penton 4: 5′ TCA GCG CGC TTC TCC TCA GCC CGT GTG GCG CTG GTG CCC GCG TAG TCG GGC ACG TCG TAG GGG TAG CCG CGG C 3′ (SEQ ID NO. 60). The complementary oligonucleotides penton 1 and penton 2 were annealed to each other as were penton 3 and penton 4. The duplex generated by annealing penton 3 and penton 4 encoded the substitution of amino acids 337 through 344 described above. The duplex generated by annealing penton 1 and penton 2 possessed a 5 base 5′ overhang which was compatible to a 5 base 5′ overhang on the duplex generated by annealing penton 3 and penton 4. The opposite end of the duplex generated by annealing penton 1 and penton 2 contained an Earl compatible overhang. The opposite end of the duplex generated by annealing penton 3 and penton 4 contained a BbvCI compatible overhang. The two duplexes were ligated to each other and ligated back into the pGEMpen5 backbone as follows. First, pGEMpen5 was digested with BbvCI and PstI and the resulting DNA fragments were separated by electrophoresis on an agarose gel. The 3360 bp fragment was excised from the gel and purified. The plasmid pGEMpen5 was also digested with PstI and Earl and the resulting fragments were separated by electrophoresis on an agarose gel. The 955 bp fragment was excised from the gel and purified. These two fragments from the pGEMpen5 plasmid were ligated with the two pairs of annealed oligonucleotides to generate the plasmid pGEMpen5PD1.

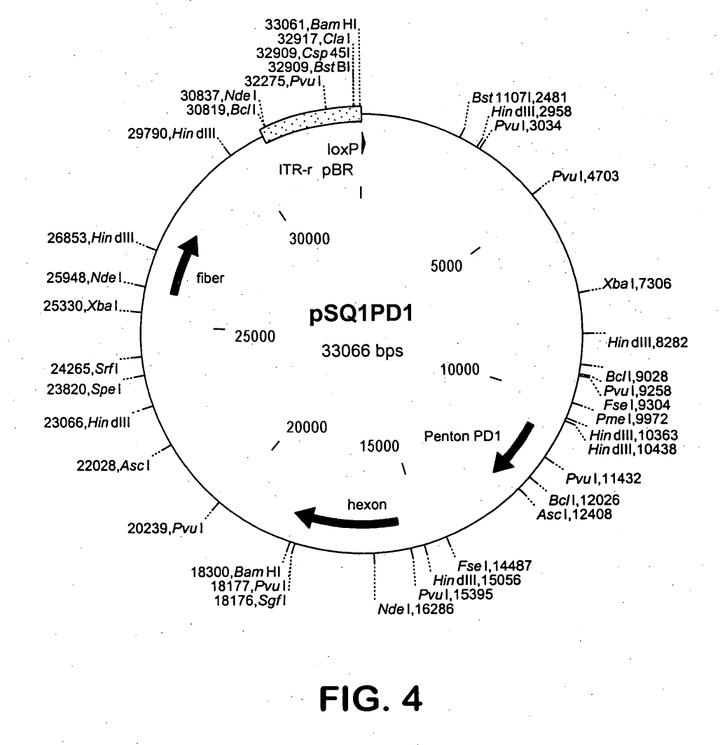

The mutated penton gene was transferred from pGEMpen5PD1 to pSQ1 using a 5-way ligation as follows. First, the region of the penton gene containing the PD1 mutation was excised from pGEMpen5PD1 by digestion with Pvul and Ascl. The 974 bp fragment containing the PD1 mutation was purified. Four DNA fragments were prepared from the pSQ1 plasmid (FIG. 3B) as follows. The plasmid was digested with Csp45I and FseI and the 9465 bp fragment was purified. In addition pSQ1 was digested with FseI and PvuI and the 2126 bp fragment was purified. The plasmid pSQ1 was digested with AscI and BamHI and the 5891 bp fragment was purified. Finally, pSQ1 was digested with BamHI and Csp451 and the 14610 bp fragment was purified. The 5 purified DNA fragments were ligated to each other to form the plasmid pSQ1PD1 (FIG. 4).

To generate adenoviral vector, pSQ1PD1 was linearized by digestion with ClaI and co-transfected into PerC6 cells with pAdmireRSVnBg (FIG. 3A) which had been digested with SalI and PacI. hexadimethrine bromide was maintained in the medium at 4 μg/ml. When a cytopathic effect was observed, a crude viral lysate was further expanded on PerC6 cells. The virus was purified by standard CsCl centrifugation procedures.

### Av1nBgFKO1PD1

Genetic Incorporation of the Fiber KO1 or KO12 Mutation in Combination with the Penton PD1 Mutation to Generate Av1 nBgFKO1PD1

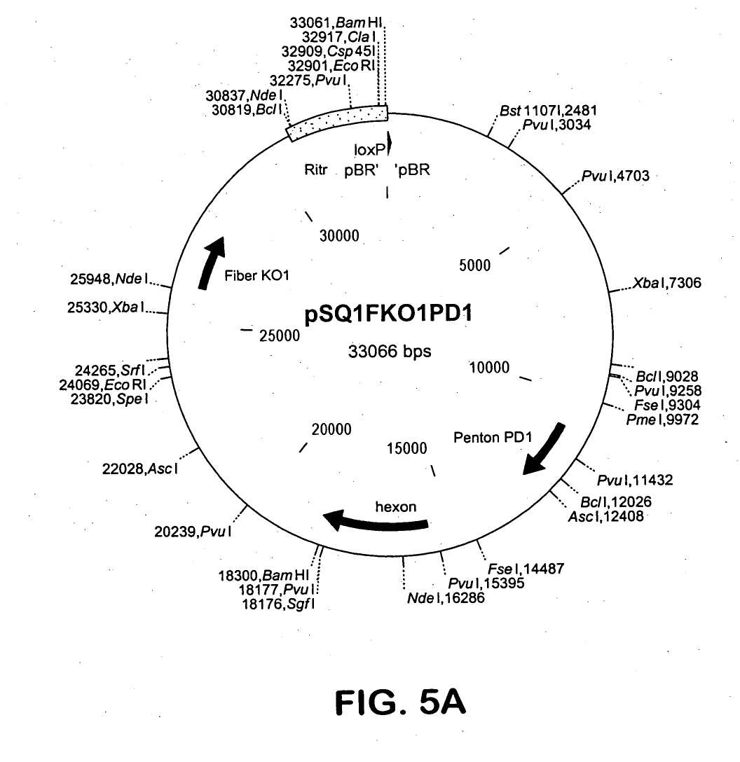

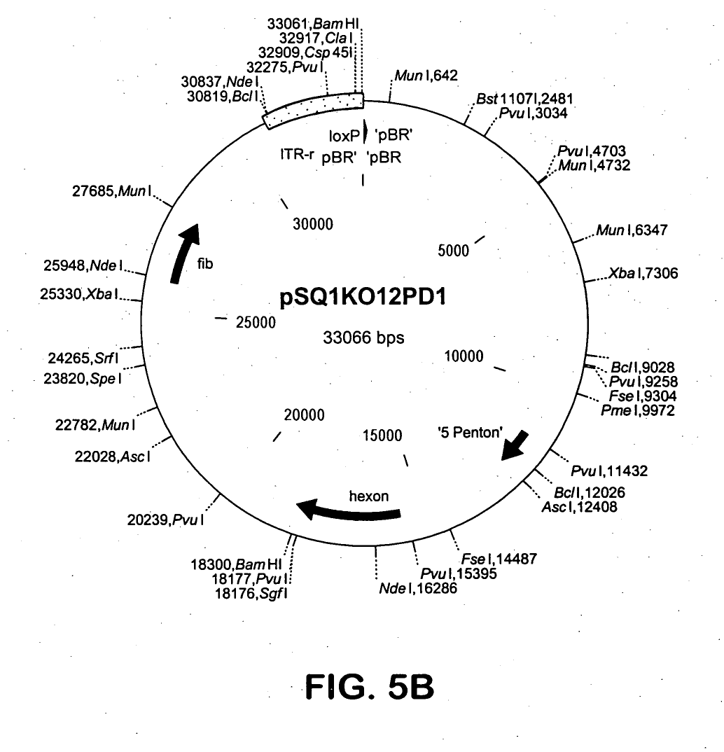

The adenoviral vectors Av1nBgFKO1PD1 and Av1nBgKO12PD1 were generated in an E1-, E3-deleted adenovirus serotype 5 genome. Both vectors contains a RSV promoted nuclear-localizing β-galactosidase gene in the E1 region and also contains either the KO1 or KO12 mutation in the fiber gene as well as the PD1 mutation in the penton gene. The vectors were constructed as follows. First, the plasmid pSQ1 PD1 was digested with Csp451 and SpeI and the 23976 bp fragment containing the PD1 mutated penton gene was purified. In addition, the plasmids pSQ1KO1 or pSQ1KO12 (FIG. 3B) were digested with Csp451 and SpeI and the 9090 bp fragment containing the KO1 or KO12 mutated fiber gene were purified. The appropriate purified fragments were ligated to each other to from the plasmid pSQ1 FKO1PD1 (FIG. 5A) or pSQ1KO12PD1 (FIG. 5B) that contains the KO1 (or KO12) mutated fiber gene and the PD1 mutated penton gene. To generate virus, pSQ1FK01PD1 or pSQKO12PD1 was linearized with ClaI and co-transfected into 633 cells with pAdmireRSVnBg (FIG. 3A) which had been digested with SalI and PacI. After three rounds of amplification in 633 cells a cytopathic effect was observed and the crude viral lysate was then amplified on PerC6 cells. Hexadimethrine bromide was maintained in the medium at 4 μg/ml. Each virus was purified by standard CsCl centrifugation procedures.

### Example 2

### In Vitro Evaluation of Adenoviral Vectors Containing the KO1 and PD1 Mutations

Several recombinant adenoviral vectors were used in these studies to demonstrate the function of the KO1 fiber mutation and included Av1nBg, Av1nBgFKO1, Av1nBgPD1, and Av1nBgFKO1PD1, described above. The transduction efficiencies of adenoviral vectors containing the KO1 and/or PD1 mutations were evaluated on cells of the alveolar epithelial cell line A549. The transduction efficiencies were compared to that of Av1nBg, an adenoviral vector containing wild type fiber and penton.

The day prior to infection, cells were seeded into 24-well plates at a density of approximately 1×10⁵ cells per well. Immediately prior to infection, the exact number of cells per well was determined by counting a representative well of cells. Each of the vectors, Av1nBg, Av1nBgFKO1, and Av1nBgFKO1PD1 were used to transduce A549 cells at each of the following particle per cell (PPC) ratios: 100, 500, 1000, 2500, 5000, 10,000. The cell monolayers were stained with X-gal 24 hours after infection and the percentage of cells expressing β-galactosidase was determined by microscopic observation and counting of cells. Transductions were done in triplicate and three random fields in each well were counted, for a total of nine fields per vector.

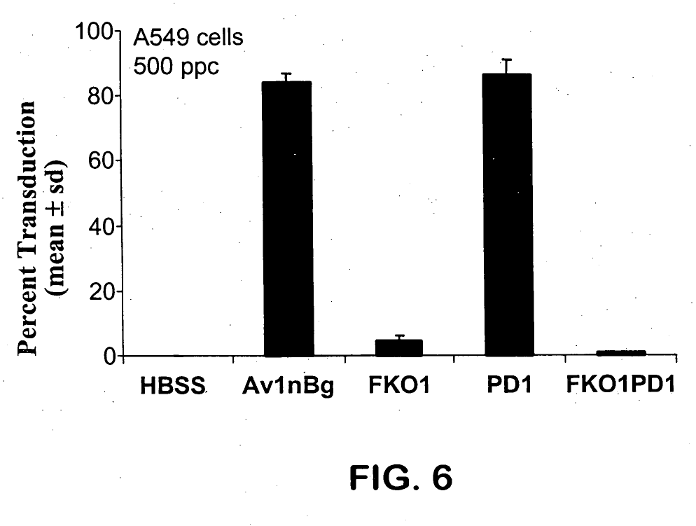

The results at the 500 PPC ratio are shown in FIG. 6 and show a significantly reduced transduction efficiency on A549 cells using vectors containing the KO1 mutation alone or when combined with PD1 compared to Av1nBg. The vectors containing the PD1 mutation alone had no effect on adenoviral transduction of A549 cells in vitro.

### Example 3

### In Vivo Analysis of Adenoviral Vectors Containing the FKO1 and PD1 Mutations

This Example provides experiments that evaluate the in vivo biodistribution of adenoviral vectors containing the KO1 and PD1 mutations and their influence on adenoviral-mediated liver transduction. The results show that ablating the viral interaction with CAR and/or integrins is not sufficient to fully detarget adenoviral vectors from the liver in vivo.

A positive control cohort received Av1nBg and a negative control group received HBSS. Additionally, the Av1nBgFKO12 and Av1nBgFKO12PD1 vectors were analyzed in vivo. These vectors each contain a fiber protein with the four amino acid substitution in the AB loop. Additionally, Av1 nBgFKO12PD1 contains a mutation in the penton base. Both of these mutations were known (see, Einfeld et al. (2001) J. Virology 75:11284-11291), and were alleged to decrease liver transduction 10 to 700 fold, respectively. Cohorts of five C57BL/6 mice received each vector via tail vein injection at a dose of 1×10¹³ particles per kg. The animals were sacrificed approximately 72 hours after vector administration by carbon dioxide asphyxiation. Liver, heart, lung, spleen, and kidney were collected from each animal. The median lobe of the liver was placed in neutral buffered formalin to preserve the sample for β-galactosidase immunohistochemistry. In addition, tissue from each organ was frozen to preserve it for hexon PCR analysis to determine vector content. A separate sample of liver from each mouse was frozen to preserve it for a chemiluminescent β-galactosidase activity assay.

For β-galactosidase immunohistochemistry slices of liver, approximately 2-3 mm thick, were placed in 10% neutral buffered formalin. After fixation, these samples were embedded in paraffin, sectioned, and analyzed by immunohistochemistry for β-galactosidase expression. A 1:1200 dilution was used of a rabbit anti-β-galactosidase antibody (ICN Pharmaceuticals, Inc.; Costa Mesa, Calif.) in conjunction with a Vectastain ABC kit (Vector Laboratories, Inc., Burlingame, Calif.) to visualize positive cells.

The chemiluminescent β-galactosidase activity assay was performed using the Galacto-Light Plus™ chemiluminescent assay (Tropix, Inc., Foster City, Calif.) system. Tissue samples were collected in lysis matrix tubes containing two ceramic spheres (Bio101, Carlsbad, Calif.) and frozen on dry ice. The tissues were thawed and 500 μl of lysis buffer from the Galacto-Light Plus kit was added to each tube. The tissue was homogenized for 30 seconds using a FastPrep System (Bio101, Carlsbad, Calif.). Liver samples were homogenized for an additional 30 seconds. β-galactosidase activity was determined in the liver homogenates according to the manufacture's protocol.

For hexon PCR analysis DNA from tissues was isolated using the Qiagen Blood and Cell Culture DNA Midi or Mini Kits (Qiagen Inc., Chatsworth, Calif.). Frozen tissues were partially thawed and minced using sterile disposable scalpels. Tissues were then lysed by incubation overnight at 55° C. in Qiagen buffer G2 containing 0.2 mg/ml RNaseA and 0.1 mg/ml protease. Lysates were vortexed briefly and then applied to Qiagen-tip 100 or Qiagen-tip 25 columns. Columns were washed and DNAs were eluted as described in the manufacturer's instructions. After precipitation, DNAs were dissolved in water and the concentrations were spectrophotometrically determined (A260 and A280) on a DU-600 (Beckman Coulter, Inc.; Fullerton, Calif.) or a SPECTRAmax PLUS (Molecular Devices, Inc.; Sunnyvale, Calif.) spectrophotometer. 2.3.2.

PCR primers and a Taqman probe specific to adenovirus hexon sequences were designed using Primer Express software v. 1.0 (Applied Biosystems, Foster City, Calif.). Primer and probe sequences were:

Hexon Forward Primer (SEQ ID NO. 61):
5′-CTTCGATGATGCCGCAGTG-3′
Hexon Reverse Primer (SEQ ID NO. 62):
5′-GGGCTCAGGTACTCCGAGG-3′
Hexon Probe (SEQ ID NO. 63):
5′-FAM-TTACATGCACATCTCGGGCCAGGAC-TAMRA-3′

Amplification was performed in a reaction volume of 50 μl under the following conditions: 10 ng (tumor) or 1 μg (liver and lung) of sample DNA, 1× Taqman Universal PCR Master Mix (Applied Biosystems), 600 nM forward primer, 900 nM reverse primer and 100 nM hexon probe. Thermal cycling conditions were: 2 minute incubation at 50° C., 10 minutes at 95° C., followed by 35 cycles of successive incubation at 95° C. for 15 seconds and 60° C. for 1 minute. Data was collected and analyzed using the 7700 Sequence Detection System software v. 1.6.3 (Applied Biosystems). Quantification of adenovirus copy number was performed using a standard curve that includes dilutions of adenovirus DNA from 1,500,000 copies to 15 copies in the appropriate background of cellular genomic DNA. For analysis of tumor tissues, a standard curve in a background of 10 ng human DNA was generated. For analysis of mouse liver and lung tissues, a standard curve using the same adenovirus DNA dilutions in a background of 1 μg CD-1 mouse genomic DNA was generated. Samples were amplified in triplicate, and the average number of total copies was normalized to copies per cell based on the input DNA weight amount and a genome size of 6×10⁹ bp.

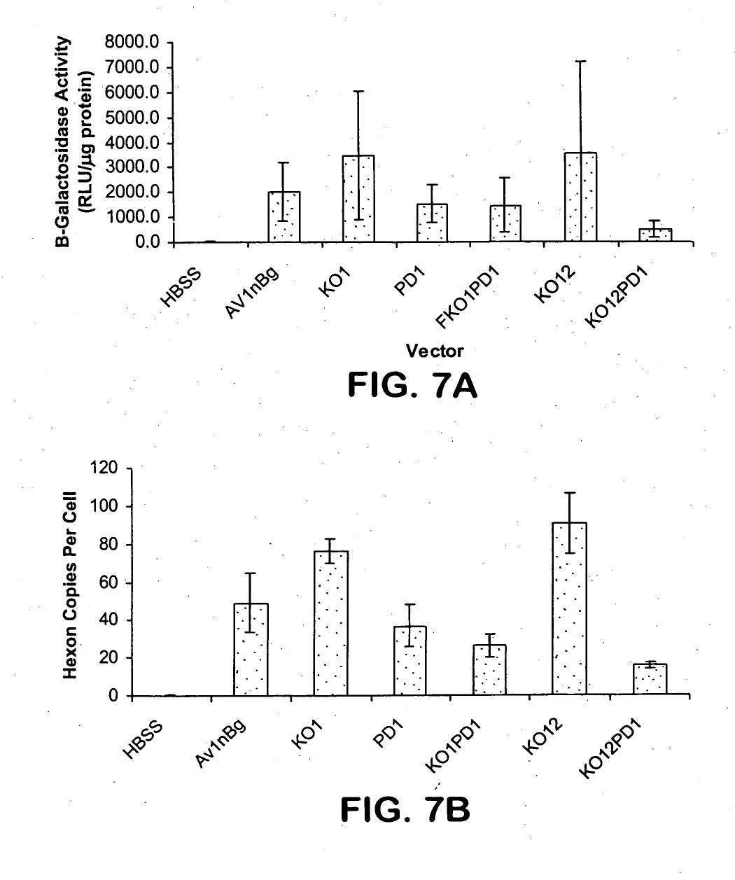

The results of the β-galactosidase activity assay and adenoviral hexon DNA content for liver transduction by these vectors are shown in FIGS. 7A and 7B. The vector containing the KO1 or KO12 mutations alone showed, on average, a slight increase in liver transduction compared to Av1nBg, which is consistent with several previous experiments. The vectors containing the PD1 mutation alone or combined with KO1 or KO12 showed a slight decrease in liver transduction compared to Av1nBg, suggesting that integrins are involved to some extent in hepatic uptake of the adenoviral vectors.

The results of the immunohistochemical staining of liver sections for β-galactosidase were consistent with the activity assays (data not shown) and demonstrate that gene expression was localized specifically to hepatocytes. The vectors containing the KO1 or KO12 mutation alone showed a slight increase in liver transduction as revealed by a more intense and frequent immunohistochemical-staining pattern. The vectors containing the PD1 mutation, either alone or combined with KO1 or KO12, showed little difference in transduction compared to Av1nBg. These results demonstrate that ablating the viral interaction with CAR and/or integrins is not sufficient to fully detarget adenoviral vectors from the liver in vivo.

In summary, the fiber AB loop mutation contained in Av1nBgFKO1 or Av1nBgKO12 ablates interaction with human and mouse CAR in vitro and diminished transduction in vitro. In vivo, however, fiber AB loop mutations behaved unexpectantly, because such mutations were found to enhance adenoviral-mediated gene transfer to liver and results in increasing vector potency. The penton base, PD1 mutation that ablates interaction with the second receptor involved in adenoviral internalization had no effect in vitro and little to no effect in vivo. These studies indicated that other receptors are responsible for adenoviral gene transfer to the liver in vivo.

### Example 4

### Description of Adenoviral Vectors Containing a Fiber with Amino Acid Substitutions at the Heparin Sulfate Binding Domain in the Fiber Shaft

Vectors containing substitutions at all four of the amino acids in the four amino acid motif in the Ad5 fiber shaft (residues 91 to 94, KKTK; SEQ ID NO. 45) were generated in order to ablate the potential interaction with HSP. The mutation is termed HSP because it potentially eliminates binding to heparan sulfate proteoglycans. Vectors containing the HSP mutation alone and combined with the KO1 mutation (fiber knob AB loop mutation that ablates CAR binding), the PD1 mutation (penton mutation that eliminates RGD/integrin interaction), and a triple knockout vector (HSP, KO1, PD1) were generated.

Generation of the HSP fiber mutation: The HSP mutation was incorporated into the fiber gene by using a PCR-based strategy of gene splicing by overlap extension (PCR SOEing). First, a segment of the Ad5 genome extending from within the E3 region into the 5′ end of the fiber gene was amplified by PCR using the plasmid pSQ1 (FIG. 3B) as a template and two primers termed 5FF and 5HSPR. The DNA sequence of 5FF is as follows: 5′ GAA CAG GAG GTG AGC TTA GA 3′ (SEQ ID NO. 53). This sequence corresponds to base pairs 25,199-25,218 of pSQ1. The DNA sequence of 5HSPR is as follows: 5′ GGC TCC GGC TCC GAG AGG TGG GCT CAC AGT GGT TAC ATT T 3′ (SEQ ID NO. 64). 5HSPR is a reverse primer for 5FF and corresponds to a region in the fiber shaft adjacent to the KKTK (SEQ ID NO. 45) region. The primer contains a 5′ extension that encodes a GAGA substitution for the native KKTK (encoded by SEQ ID NO. 45) amino acid sequence. A second PCR using pSQ1 as a template amplified the region immediately 3′ of the KKTK (SEQ ID NO. 45) site and extending past the MunI site located 40 base pairs 3′ of the stop codon for the fiber gene. The two primers used for this reaction were 3HSPF and 3FR. The DNA sequence of 3HSPF is as follows: 5′ GGA GCC GGA GCC TCA AAC ATA AAC CTG GAA AT 3′ (SEQ ID NO. 16). It contains a 5′ extension that is complementary to the 5′ extension of 5HSPR. The DNA sequence of 3FR is as follows: 5′ GTG GCA GGT TGA ATA CTA GG 3′ (SEQ ID NO. 56).

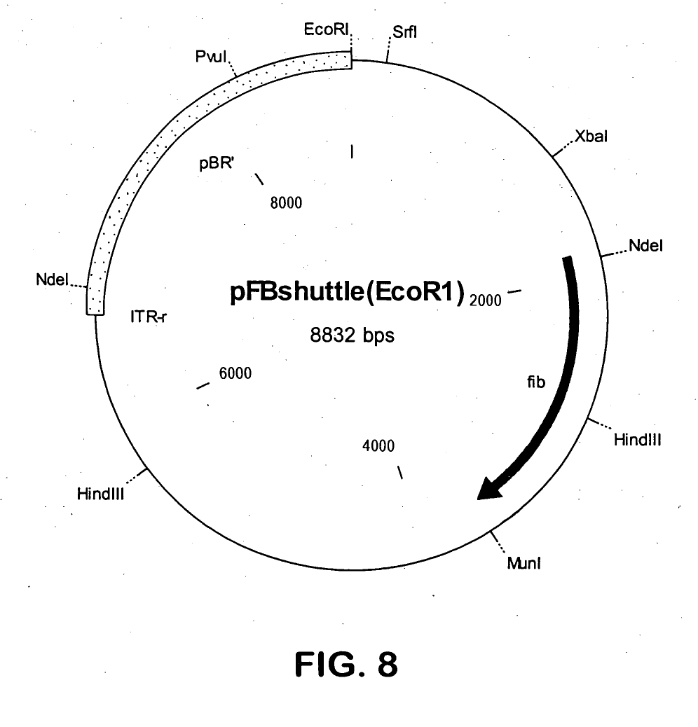

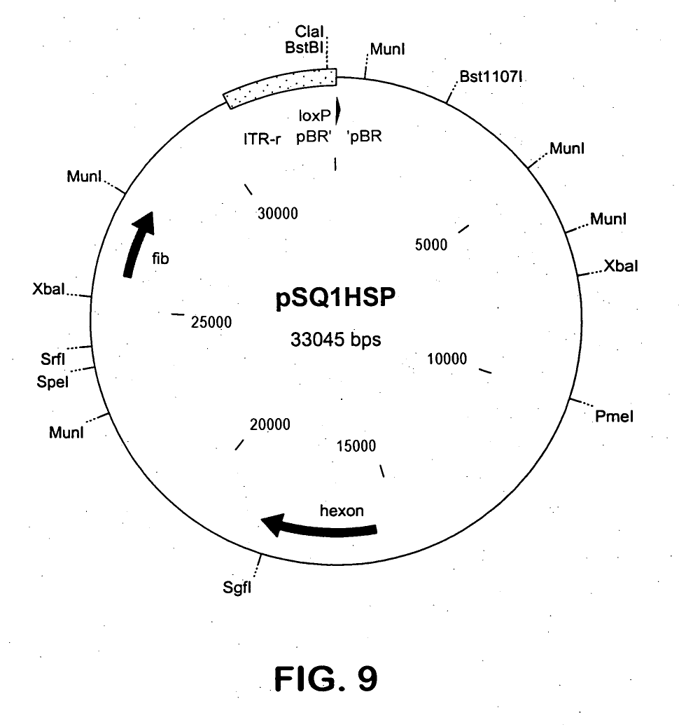

The two PCR products were joined by PCR SOEing using primers 5FF and 3FR. The resulting PCR product was digested with the restriction enzymes XbaI and MunI. The 2355 bp fragment was gel purified and ligated with the 6477 bp XbaI to MunI fragment of the plasmid pFBshuttle(EcoRI) (FIG. 8) to generate the plasmid pFBSEHSP. The plasmid pFBshuttle(EcoRI) was generated by digesting the plasmid pSQ1 with EcoRI, then gel purifying and self-ligating the 8.8 kb fragment containing the fiber gene. Next, the fiber gene containing the HSP mutation was transferred from pFBSEHSP into pSQ1 using a three-way ligation. The 16,431 bp EcoRI to NdeI fragment of pSQ1, the 9043 bp NdeI to XbaI fragment of pSQ1, and the 7571 bp XbaI to EcoRI fragment of pFBSEHSP were isolated and ligated to generate pSQ1 HSP (FIG. 9).

To generate a recombinant adenoviral vector containing the HSP mutation in the fiber gene, pSQ1 HSP was digested with ClaI and pAdmireRSVnBg (FIG. 3A) was digested with SalI and PacI, then the two digested plasmids were co-transfected into 633 cells (von Seggern et al. (2000) J Virology 74:354-362). Homologous recombination between the two plasmids generated a full-length adenoviral genome capable of replication in 633 cells, which inducibly express Ad5 E1A and constitutively express wild-type fiber protein. After propagation on 633 cells, the virus capsid contained wildtype and mutant fiber proteins. To obtain viral particles containing only the modified fiber with the HSP mutation, the viral preparation was used to infect PerC6 cells, which do not express fiber. The resulting virus, termed Av1nBgFS*, was purified by standard CsCl centrifugation procedures.

Generation of Vector Containing the HSP and KO1 Mutations

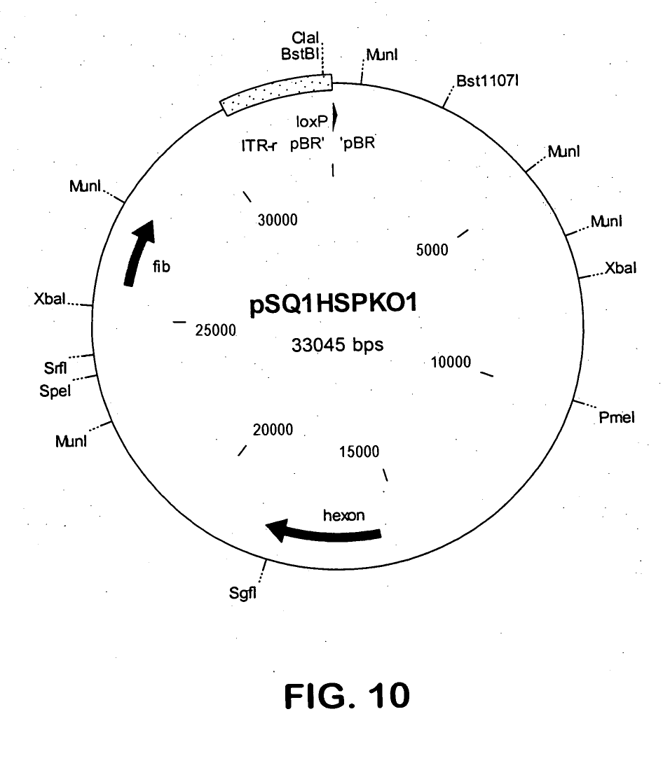

To generate an adenoviral vector containing the HSP and KO1 mutations in fiber, a PCR SOEing strategy identical to the one described above was used except that the plasmid pSQ1 FKO1 was used as the template. The PCR SOEing product was digested with XbaI and MunI and ligated with the 6477 bp XbaI to MunI fragment of pFBshuttle(EcoRI) to generate pFBSEHSPKO1. The fiber gene containing the HSP and KO1 mutations was transferred from pFBSEHSPKO1 into the pSQ1 backbone using a three-way ligation strategy identical to the one described above for the HSP mutation alone, to generate the plasmid pSQ1 HSPKO1 (FIG. 10). Recombinant adenoviral vector containing the HSP and KO1 mutations in the fiber gene was generated by co-transfecting pSQ1HSPKO1 digested with ClaI and pAdmireRSVnBg digested with SalI and PacI into 633 cells. Adenovirus was propagated and purified as described above for the vector containing the HSP mutation alone. The resulting virus was termed Av1nBgFKO1S*.

Generation of Vector Containing the HSP and PD1 Mutations

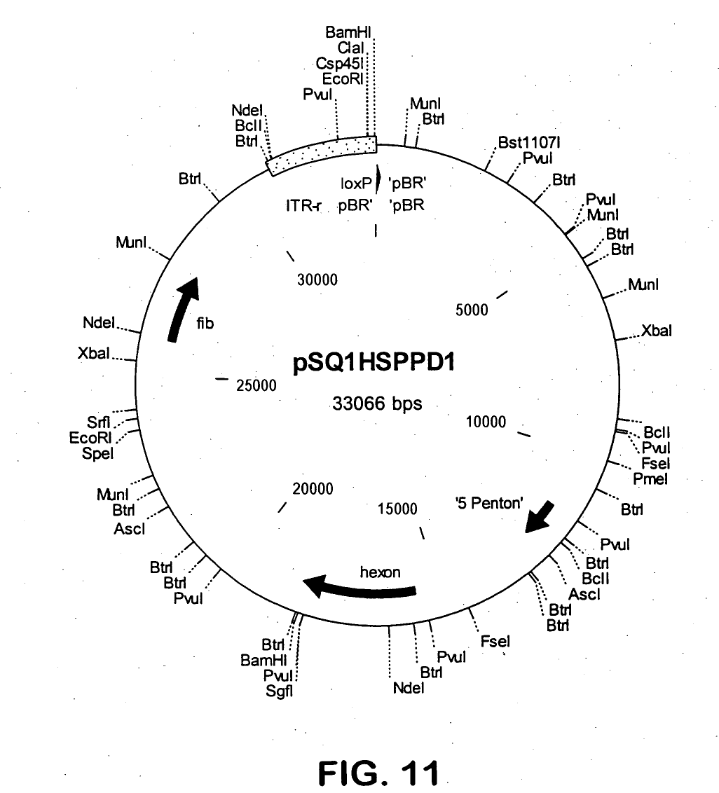

The following strategy was used to generate a recombinant adenoviral vector containing the fiber HSP mutation and the penton PD1 mutation. The plasmid pSQ1PD1 (FIG. 4) was digested with the restriction enzymes Csp451 and SpeI and the 23,976 bp fragment was isolated and purified. In addition, the plasmid pSQ1 HSP was also digested with Csp451 and SpeI and the 9090 bp fragment was isolated and purified and ligated to the 23,976 bp fragment to generate the plasmid pSQ1 HSPPD1 (FIG. 11), which contains the fiber HSP and penton PD1 mutations. An adenoviral vector was generated, propagated, and purified as described above. The resulting virus was termed Av1nBgS*PD1.

Generation of Vector Containing the HSP, KO1, and PD1 Mutations

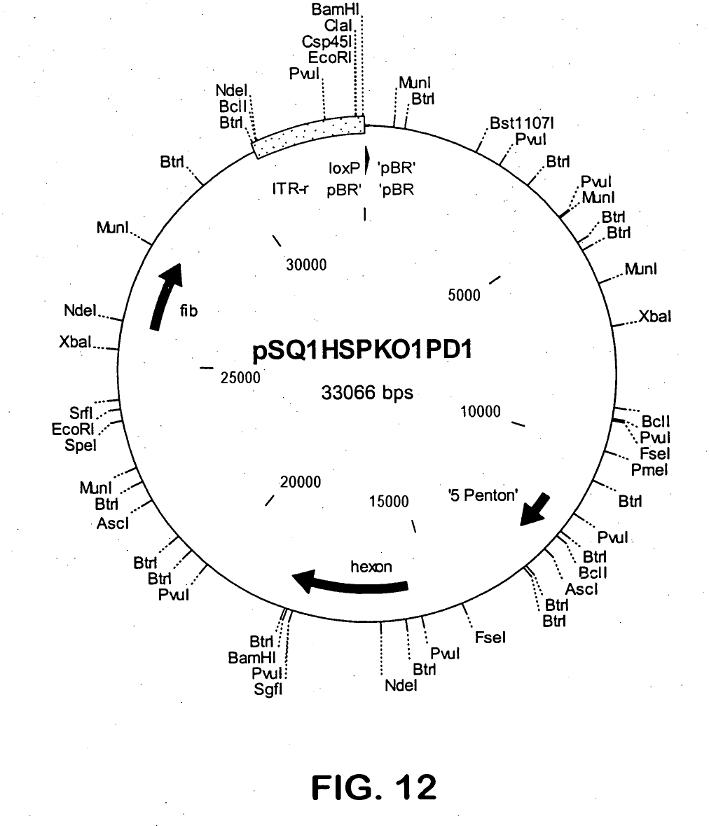

To generate an adenoviral vector containing the HSP, KO1, and PD1 mutations the following strategy was used. First, the plasmid pSQ1PD1 was digested with Csp451 and SpeI and the 23,976 bp fragment was isolated and purified. In addition, the plasmid pSQ1HSPKO1 was digested with Csp451 and SpeI and the 9090 bp fragment was isolated and purified. The two DNA fragments were ligated to form the plasmid pSQ1 HSPKO1PD1 (FIG. 12). Recombinant adenoviral vector was generated, propagated, and purified as described above. The resulting virus was termed Av1 nBgFKO1S*PD1.

### Example 5

### In Vitro Evaluation of Adenoviral Vectors Containing the HSP Fiber Mutation

The transduction efficiencies of adenoviral vectors containing the HSP mutation in the fiber gene, either alone or combined with the KO1 and/or PD1 mutations, were evaluated on A549 and HeLa cells. The transduction efficiencies were compared to that of Av1nBg, an adenoviral vector containing wild type fiber and penton. The day prior to infection, cells were seeded into 24-well plates at a density of approximately 1×10⁵ cells per well. Immediately prior to infection, the exact number of cells per well was determined by counting a representative well of cells. Each of the vectors, Av1nBg (see, Stevenson et al. (1997) J. Virol. 71:4782-4790), Av1nBgS*, Av1nBgFK01S*, Av1nBgS*PD1, and Av1nBgFK01S*PD1, were used to transduce A549 cells at each of the following particle per cell (PPC) ratios: 100, 500, 1000, 2500, 5000, 10,000. HeLa cells were transduced with each of the above vectors, as well as a vector containing the KO1 mutation alone (Av1nBgFKO1) and a vector containing the PD1 mutation alone (Av1nBgPD1) at 2000 PPC. The cell monolayers were stained with X-gal 24 hours after infection and the percentage of cells expressing β-galactosidase was determined by microscopic observation and counting of cells. Transductions were done in triplicate and three random fields in each well were counted, for a total of nine fields per vector.

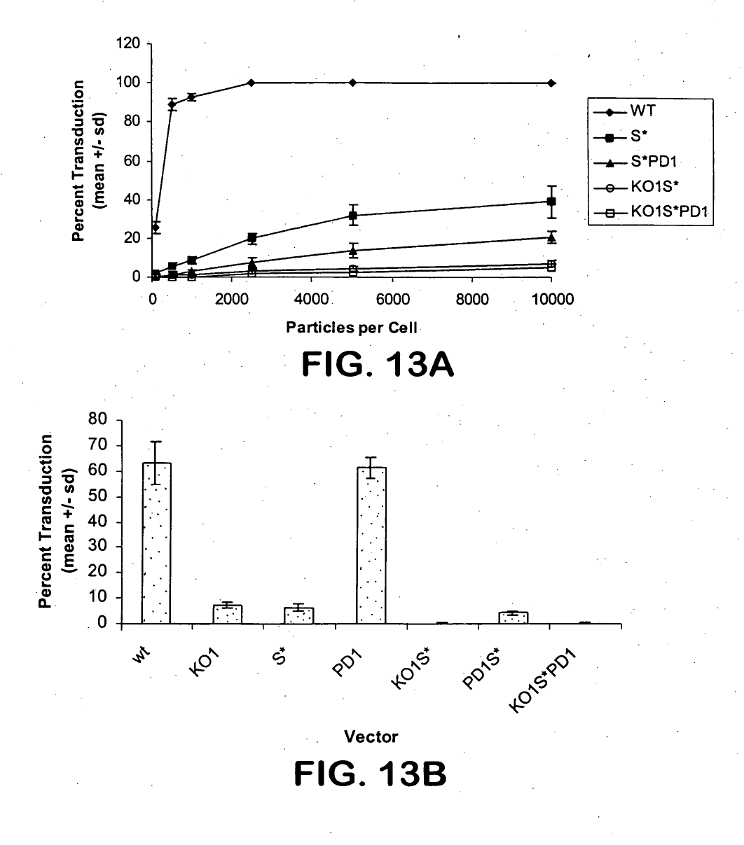

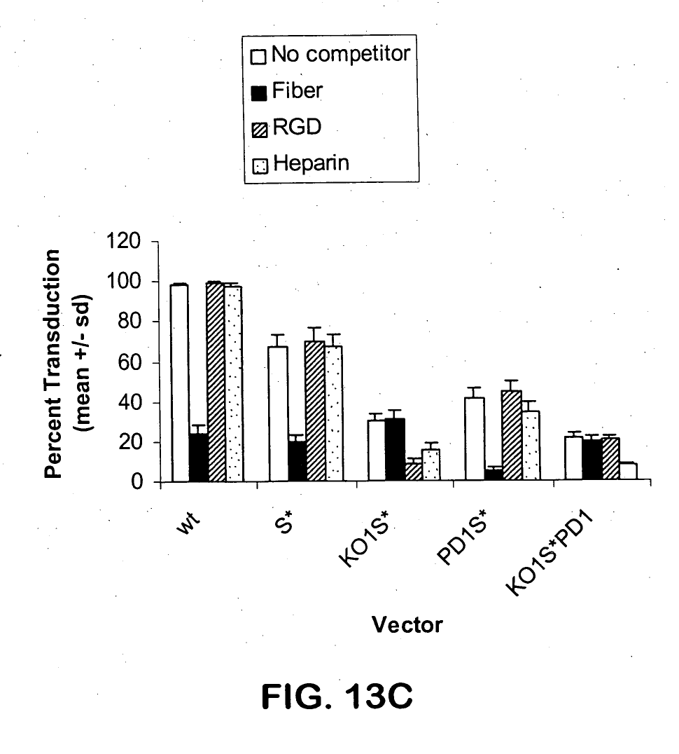

The results (depicted in FIGS. 13A-13B) showed significantly reduced transduction efficiencies on A549 and HeLa cells using vectors containing the HSP mutation compared to Av1nBg. The vectors containing the HSP mutations, however, demonstrated a dose response on A549 cells, in that increasing PPC ratios yielded increasing transduction.

Competition experiments were done to determine which receptor molecular interactions are involved in transduction of A549 cells by the various vectors. Transductions were performed in the presence or absence of various competitors including Ad5 fiber knob, a 50 amino acid oligopeptide derived from Adenovirus serotype 2 penton base which spans the RGD tripeptide region, or heparin (Invitrogen Life Technologies, Gaithersburg, Md.). Monolayers of A549 cells were cultured in Richters medium supplemented with 10% FBS and were transduced with Av1nBg, Av11nBgS*, Av1nBgFK01S*, Av1nBgS*PD1, or Av1nBgFK01S*PD1 in infection medium (1M, Richters medium plus 2% FBS). Different PPC ratios were used for the different vectors to achieve measurable transduction levels. The PPC ratios were as follows: Av1nBg: 500 PPC, Av1nBgS*: 10,000 PPC, Av1nBgFKO1S*: 20,000 PPC, Av1nBgS*PD1: 10,000 PPC, and Av1nBgFK01S*PD1: 20,000 PPC. Fiber knob competition was performed by pre-incubating cells in IM containing 16 μg/ml of fiber knob for 10 minutes at room temperature prior to infection with virus. Penton base peptide competition was performed by pre-incubating cells in IM containing 500 nM peptide for 10 minutes at room temperature prior to infection with virus. Heparin competition was performed by pre-incubating each adenoviral vector in IM containing 3 mg/ml of heparin for 20 minutes at room temperature. In all cases, the competitor remained in the IM during the 1 hour infection when virus was rocked on the cell monolayers at 37° C. in 5% CO₂. After infection, the monolayers were washed with PBS, 1 ml of complete medium was added per well and the cells were incubated for an additional 24 hours to allow for β-galactosidase expression. The cell monolayers were then fixed and stained with X-Gal. The percentage of cells transduced was determined by light microscopy as described above. Each condition was carried out in triplicate and three random fields per well were counted, for a total of nine fields per condition. The average percentage of transduction per high-power field was determined.

The results of the competition experiment (FIG. 13C) showed that fiber knob inhibited transduction of cells by all vectors except for those that contained the KO1 mutation. The penton base peptide only inhibited transduction by Av1nBgFK01S*. Heparin inhibited transduction by Av1nBgFKO1S* and Av1nBgFKO1S*PD1, but did not affect transduction by any of the other viruses suggesting the presence of additional heparin binding sites on the adenoviral capsid but that the shaft contains the predominant site.

### Example 6

### In Vivo Analysis of Adenoviral Vectors Containing the HSP Mutation in Fiber

The objective of this study was to evaluate the in vivo biodistribution of adenoviral vectors containing the HSP mutation and to determine whether this shaft modification influences adenoviral-mediated liver transduction. In addition, vectors containing the HSP mutation combined with KO1, or PD1, or a combination of all three mutations were evaluated as well as vectors containing the KO1 mutation alone and the PD1 mutation alone. A positive control cohort received Av1nBg and a negative control group received HBSS. Cohorts of five C57BL/6 mice received each vector via tail vein injection at a dose of 1×10¹³ particles per kg. The animals were sacrificed approximately 72 hours after vector administration by carbon dioxide asphyxiation. Liver, heart, lung, spleen, and kidney were collected from each animal. The median lobe of the liver was placed in neutral buffered formalin to preserve the sample for β-galactosidase immunohistochemistry. In addition, tissue from each organ was frozen to preserve it for hexon real time PCR analysis to determine vector content. A separate sample of liver from each mouse was frozen to preserve it for a chemiluminescent β-galactosidase activity assay. β-galactosidase immunohistochemistry, hexon real-time PCR and the chemiluminescent β-galactosidase activity assay were carried out as described in Example 3.

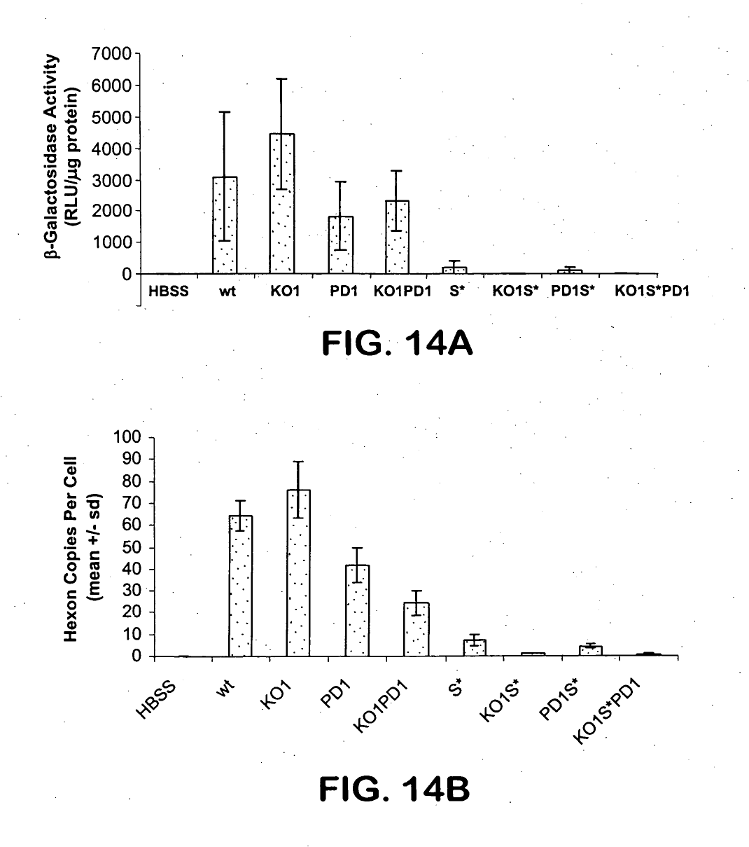

The results of the β-galactosidase activity assay (FIG. 14A) and adenoviral hexon DNA content (FIG. 14B) showed a dramatic reduction in liver transduction by vectors containing the HSP mutation. The vectors containing the HSP mutation alone resulted in reducing adenoviral-mediated liver gene expression by approximately 20-fold. When combined with the KO1 mutation (HSP, KO1, PD1), yielded approximately a 1000-fold reduction in β-galactosidase activity in the liver compared to the control vector Av1nBg. The vector containing the KO1 mutation alone showed a slight increase, on average, in liver transduction compared to Av1nBg, which is consistent with several previous experiments. The vectors containing the PD1 mutation alone or combined with KO1 showed a slight decrease in liver transduction compared to Av1nBg, although the decrease was not statistically significant. Analysis of hepatic adenoviral hexon DNA content (FIG. 14B) confirmed these results.

The results of the immunohistochemical staining of liver sections for β-galactosidase were consistent with the activity assays (data not shown) and demonstrated that gene expression was localized specifically to hepatocytes. Vectors containing the HSP mutation, either alone or in combination with KO1 and/or PD1, showed a dramatic reduction in hepatocyte transduction. The vector containing the KO1 mutation alone showed a slight increase in liver transduction as revealed by a more intense and frequent immunohistochemical staining pattern. The vectors containing the PD1 mutation, either alone or combined with KO1, showed little difference in transduction compared to Av1nBg.

### Example 7

### Description of Adenoviral Vectors Containing the HSP Fiber Shaft Mutation with and without the KO1 Fiber Mutation and with and without a cRGD Targeting Ligand in the Fiber Knob HI Loop

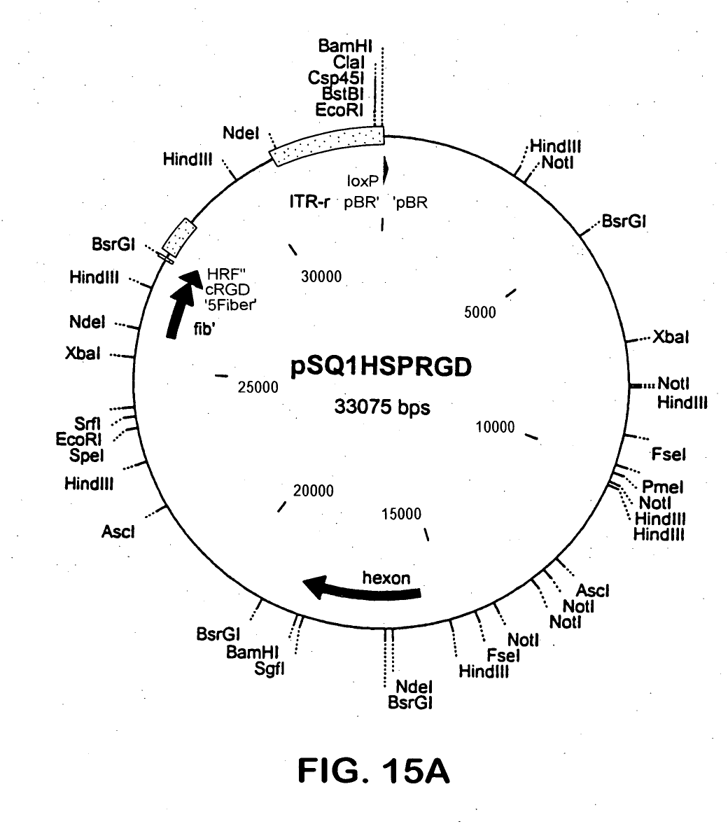

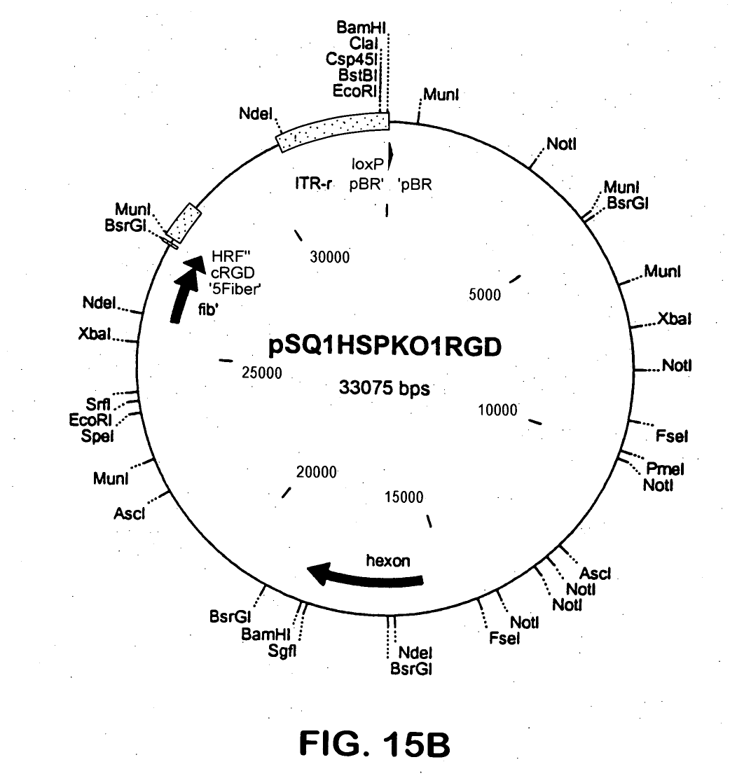

Generation of vector containing the HSP fiber shaft mutation and a cRGD ligand in the HI loop: The following strategy was used to generate an adenoviral vector containing a fiber with the HSP shaft mutation and a cRGD ligand in the HI loop. The plasmid p5FloxHRFRGD was digested with the restriction enzymes BstXI and KpnI and the 1157 bp fragment was isolated and purified. In addition, the fiber shuttle plasmid pFBSEHSP, described in Example 1 above, was digested with BstXI and KpnI and the 4549 bp and 3156 bp fragments were isolated and purified. The three fragments were ligated to generate the plasmid pFBSEHSPRGD, which encodes a fiber containing the HSP mutation and cRGD in the Hi loop. The fiber gene from this plasmid was transferred into the pSQ1 backbone as follows. The plasmid pFBSEHSPRGD was digested with EcoRI and XbaI and the 7601 bp fragment was isolated and purified. The plasmid pSQ1 (FIG. 3B) was digested with the restriction enzymes EcoRI, NdeI, and XbaI and the 16,431 bp EcoRI to NdeI fragment and the 9043 bp NdeI to XbaI fragment were isolated and purified. The three DNA fragments were ligated to generate the plasmid pSQ1 HSPRGD (FIG. 15A).

To generate a recombinant adenoviral vector containing the HSP mutation in the fiber gene along with a cRGD ligand in the HI loop, the plasmid pSQ1 HSPRGD was digested with ClaI and co-transfected into 633 cells with pAdmireRSVnBg which had been digested with SalI and PacI. After propagation on 633 cells, the virus capsid contained wildtype and mutant fiber proteins. To obtain viral particles containing only the modified fiber with the HSP mutation and a cRGD ligand, the viral preparation was used to infect PerC6 cells, which do not express fiber. The resulting virus, termed Av1nBgS*RGD, was purified by standard CsCl centrifugation procedures.

### Generation of Vector Containing the HSP Fiber Shaft Mutation, the KO1 Fiber Knob Mutation, and a cRGD Ligand in the HI Loop

The following strategy was used to generate an adenoviral vector containing a fiber with the HSP shaft mutation, the KO1 fiber knob mutation, and a cRGD ligand in the HI loop. The plasmid p5FloxHRFRGD was digested with the restriction enzymes BstXI and KpnI and the 1157 bp fragment was isolated and purified. In addition, the fiber shuttle plasmid pFBSEHSPKO1, described in Example 1 above, was digested with BstXI and KpnI and the 4549 bp and 3156 bp fragments were isolated and purified. The three fragments were ligated to generate the plasmid pFBSEHSPKO1 RGD, which encodes a fiber containing the HSP mutation, the KO1 mutation, and cRGD in the HI loop. The fiber gene from this plasmid was transferred into the pSQ1 backbone as follows. The plasmid pFBSEHSPKPO1RGD was digested with EcoRI and XbaI and the 7601 bp fragment was isolated and purified. The plasmid pSQ1 (FIG. 3B) was digested with the restriction enzymes EcoRI, NdeI, and XbaI and the 16,431 bp EcoRI to NdeI fragment and the 9043 bp NdeI to XbaI fragment were isolated and purified. The three DNA fragments were ligated to generate the plasmid pSQ1 HSPKO1 RGD (FIG. 15B).

To generate a recombinant adenoviral vector containing the HSP and KO1 mutations in the fiber gene along with a cRGD ligand in the HI loop, the plasmid pSQ1 HSPKO1 RGD was digested with ClaI and co-transfected into 633 cells with pAdmireRSVnBg which had been digested with SalI and PacI. After propagation on 633 cells, the virus capsid contained wildtype and mutant fiber proteins. To obtain viral particles containing only the modified fiber with the HSP and KO1 mutations and a cRGD ligand, the viral preparation was used to infect PerC6 cells, which do not express fiber. The resulting virus, termed Av1nBgFKO1S*RGD, was purified by standard CsCl centrifugation procedures.

### Example 8

### In Vitro Evaluation of Adenoviral Vectors Containing the HSP Fiber Shaft Mutation with or without the Fiber Knob KO1 Mutation and with or without a cRGD Ligand in the HI Loop

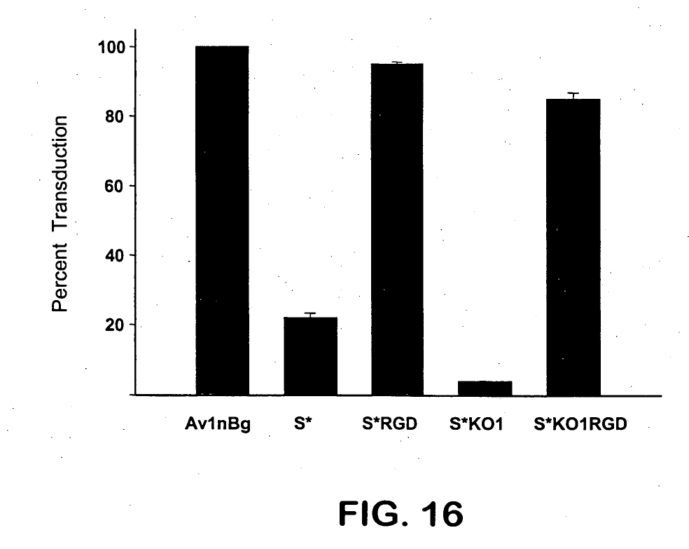

The transduction efficiencies of adenoviral vectors containing the HSP fiber shaft mutation with or without the fiber KO1 mutation and with or without the cRGD ligand in the HI loop were evaluated on A549 cells. The transduction efficiencies were compared to that of Av1nBg, an adenoviral vector containing wild type fiber. The day prior to infection, cells were seeded into 24-well plates at a density of approximately 1×10⁵ cells per well. Immediately prior to infection, the exact number of cells per well was determined by counting a representative well of cells. Each of the vectors, Av1nBg, Av1nBgS*, Av1nBgFKO1S*, Av1nBgS*RGD, and Av1nBgFKO1S*RGD, were used to transduce A549 cells at a particle to cell ratio of 6250. The cell monolayers were stained with X-gal 24 hours after infection and the percentage of cells expressing β-galactosidase was determined by microscopic observation and counting of cells. Transductions were done in triplicate and three random fields in each well were counted, for a total of nine fields per vector. The results (FIG. 16) showed that the cRGD ligand dramatically increased the transduction efficiencies of vectors containing the HSP mutation alone or combined with the KO1 mutation. Av1nBgS* yielded approximately 22% positive cells, while Av1 nBgS*RGD yielded approximately 95% positive cells. Similarly, Av1nBgFKO1S* yielded only 4% positive cells, while Av1nBgFKO1S*RGD yielded 85% positive cells. Therefore, the vector containing the shaft mutation is viable and can be retargeted with the addition of a ligand.

### Example 9

### Construction of Ad5 Vectors Containing the Ad35 Fiber and Derivatives Thereof

The KO1 and HSP mutations in the Ad5 fiber protein (5F), described above, were designed to ablate interactions that are responsible for the normal tropism of the Ad5 virus. An alternative strategy to detarget the virus is to replace the Ad5 fiber with a fiber from another serotype which does not bind CAR and which does not possess the heparin sulfate proteoglycan (HSP) binding domain (KKTK; SEQ ID NO. 45) within the shaft. The fiber of adenovirus serotype 35 (35F) does not bind CAR and does not possess the HSP binding domain in its shaft. Replacement of the 5F with the 35F can detarget the liver and provide a suitable platform for retargeting the vector to the desired tissue.

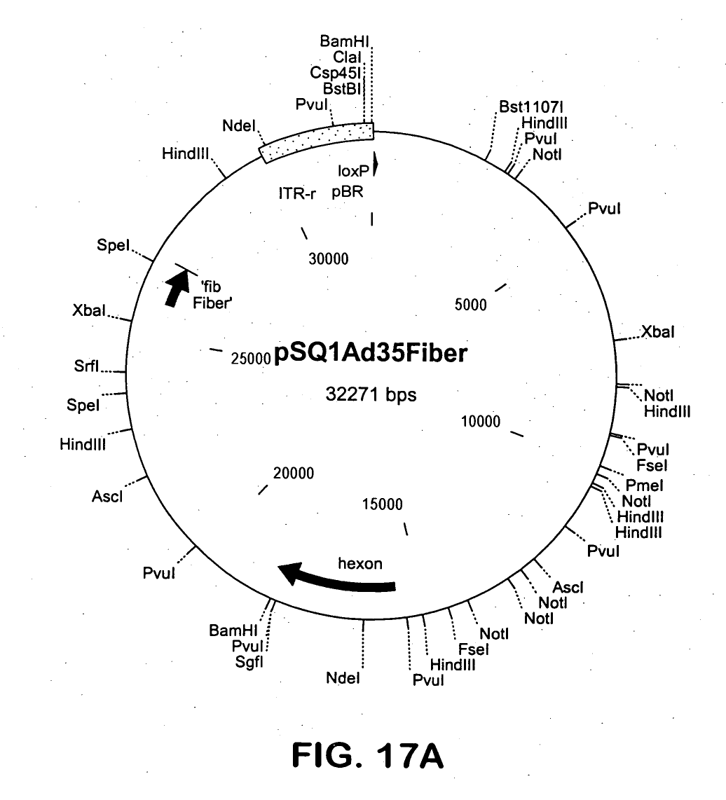

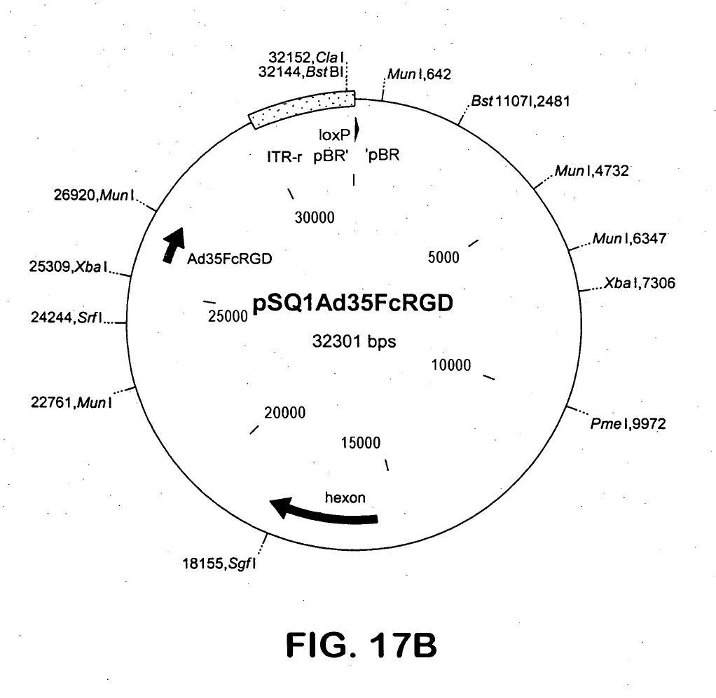

Generation of an Ad5 based vector containing the Ad35 fiber: A PCR SOEing strategy was used to generate a vector based on the Ad5 serotype but containing the Ad35 fiber in place of the Ad5 fiber. First, PCR was used to amplify a region in the plasmid pSQ1 between the XbaI site at bp 25,309 and the start of the fiber gene. The primers used for this reaction were P-0005/U and P-0006/L. The DNA sequence of P-0005/U was as follows: 5° C. TCT AGA AAT GGA CGG AAT TAT TAC AG 3′ (SEQ ID NO. 65). This sequence corresponds to bp 25,308 through 25,334 of pSQ1. The DNA sequence of P-0006/L was as follows: 5′ TCT TGG TCA TCT GCA ACA ACA TGA AGA TAG TG 3′ (SEQ ID NO. 66). It contains a 10 base pair 5′ extension that is complementary to the start of the Ad35 fiber gene, while the remainder of the primer anneals to the sequence immediately 5′ of the ATG start codon of the fiber gene in pSQ1. A PCR product of the expected size, 583 bp, was obtained and the DNA was gel purified. A second PCR amplified the Ad35 fiber gene using DNA extracted from wildtype Ad35 virus as a template. The primers used for this reaction were P-0007/U and 35FMun. The DNA sequence of P-0007/U was as follows: 5′ GT TGT TGC AG ATG ACC AAG AGA GTC CGG CTC A 3′ (SEQ ID NO. 67). It contains a 10 base pair 5′ extension that is homologous to the 10 bp immediately prior to the ATG start codon of the fiber gene in Ad5. The remainder of the primer anneals to the start of the Ad35 fiber gene. The DNA sequence of 35FMun was as follows: 5′ AG CAA TTG AAA AAT AAA CAC GTT GAA ACA TAA CAC AAA CGA TTC TTT A GTT GTC GTC TTC TGT AAT GTA AGA A 3′ (SEQ ID NO. 68). It contains a 46 base pair 5′ extension that is complementary to the region of the Ad5 genome between the end of fiber and the MunI site 40 bp downstream of the fiber gene. In addition, the 5′ extension encodes the last amino acid and stop codon of the Ad5 fiber gene. This region was retained in the vector because it contains the polyadenylation site for the fiber gene. The remainder of the primer anneals to the 3′ end of the Ad35 fiber gene, up to the next to last amino acid codon. A PCR product of the expected size, 1027 bp, was obtained and the DNA was gel purified. The two PCR products were mixed and joined together by PCR SOEing using primers P-0005/U and P-0009. The DNA sequence of P-0009 was as follows: 5′ AG CAA TTG AAA AAT AAA CAC GTT G 3′ (SEQ ID NO. 69). It corresponds to bp 27,648 through 27,669 of pSQ1 and overlaps the MunI site in that region. A PCR product of the expected size, 1590 bp, was obtained and gel purified. It was cloned into the plasmid pCR4blunt-TOPO (Invitrogen Corporation, Carlsbad Calif.) using the Zero Blunt TOPO PCR Cloning Kit from Invitrogen. This intermediate cloning step simplified DNA sequencing of the PCR SOEing product. The resulting plasmid, termed pTOPOAd35F, was digested with XbaI and MunI and the 1585 bp digestion product was gel purified and ligated with the 6477 bp fragment of pFBshuttle(EcoRI) digested with XbaI and MunI to generate the plasmid pFBshuttleAd35F. The Ad35 fiber gene was transferred from pFBshuttleAd35F into pSQ1 as follows. The plasmid pSQ1 was digested with EcoRI and the 24,213 bp fragment was gel purified. The plasmid pFBshuttleAd35F was linearized with EcoRI and ligated with the 24,213 bp fragment from pSQ1. Restriction diagnostics were performed to screen for constructs containing the Ad35 fiber gene inserted into the pSQ1 backbone in the correct orientation. The pSQ1 plasmid containing the Ad35 fiber gene in the proper orientation was termed pSQ1Ad35Fiber (FIG. 17A). To generate adenoviral vector containing the Ad35 fiber, pSQ1Ad35Fiber was digested with ClaI and co-transfected into 633 cells with pAdmireRSVnBg which had been digested with SalI and PacI. After propagation on 633 cells, the resulting virus contained Ad5 fiber and Ad35 fibers on its capsid. The virus was amplified on PerC6 cells to generate virus containing only the Ad35 fiber on its capsid. The resulting virus preparation was termed Av1nBg35F.

Construction of adenoviral vectors containing chimeric fibers derived from Ad5 and Ad35: Two chimeric fiber constructs were prepared by PCR gene overlap extension using plasmids containing the full length Ad5 or Ad35 fiber cDNAs as templates. The Ad5 fiber tail and shaft regions (5TS; amino acids 1 to 403) were connected with the Ad35 fiber head region (35H; amino acids 137 to 323) to form the 5TS35H chimera, and the Ad35 fiber tail and shaft regions (35TS; amino acids 1 to 136) were connected with the Ad5 fiber head region (5H; amino acids 404 to 581) to form the 35TS5H chimera. The fusions were made at the conserved TLWT sequence at the fiber shaft-head junction.

For the construction of the 5TS35H chimera, the pFBshuttle(EcoRI) plasmid was used as the template with primers P1 and P2 to generate the 5′ fragment. The 3′ fragment was generated using the pFBshuttleAd35 plasmid as the template with the P3 and P4 primers. The sequence of each primer used in the construction of these chimeric fibers is listed in Table 2. Amplified PCR products of the expected size were obtained and were gel purified. A second PCR was carried out with the end primers P1 and P4 to join the two fragments together. The DNA fragment generated in the second PCR was digested with Xba1 and Mun1 and was cloned directly into pFBshuttle(EcoRI) to create the fiber shuttle plasmid pFBshuttle5TS35H.

TABLE 2

Primers Used For The Exchange Of Fiber Shaft
Regions Between Ad5 And Ad35 Fibers

| Primer | | |
|---|---|---|
| designation | Sequence | SEQ ID |
| P1 | 5′-GAACAGGAGGTGAGCTTAGA-3′ | 70 |
| P2 | 5′-GTTAGGTGGAGGGTTTATTCCGGTCCACA | 71 |
| | AAGTTAGCTTATC-3′ | |
| P3 | 5′-GATAAGCTAACTTTGTGGACCGGAATAAA | 72 |
| | CCCTCCACCTAAC-3′ | |
| P4 | 5′-GTGGCAGGTTGAATACTAGG-3 | 73 |
| P5 | 5′-GTTAGGAGATGGAGCTGGTGTAGTCCATA | 74 |
| | AGGTGTTAATAC-3′ | |
| P6 | 5′-GTATTAACACCTTATGGACTACACCAGCT | 75 |
| | CCATCTCCTAAC-3′ | |
| P7 | 5′-TGCGCAAAAACAATCACCACGACAATCAC | 76 |
| | AATGTACATTGGAAGAAATCATACG-3′ | |
| P8 | 5′-ACATTGTGATTGTCGTGGTGATTGTTTTT | 77 |
| | GCGCATATGCCATACAATTTGAATG-3′ | |

For the construction of the 35TS5H chimera, the pFBshuttleAd35 plasmid was used as the template with the P1 and P5 primers to generate the 5′ fragment. The 3′ fragment was generated using the pFBshuttle(EcoRI) plasmid as the template with the P6 and P4 primers. Following the same procedure described above, the fiber shuttle plasmid pFBshuttle35TS5H was generated.

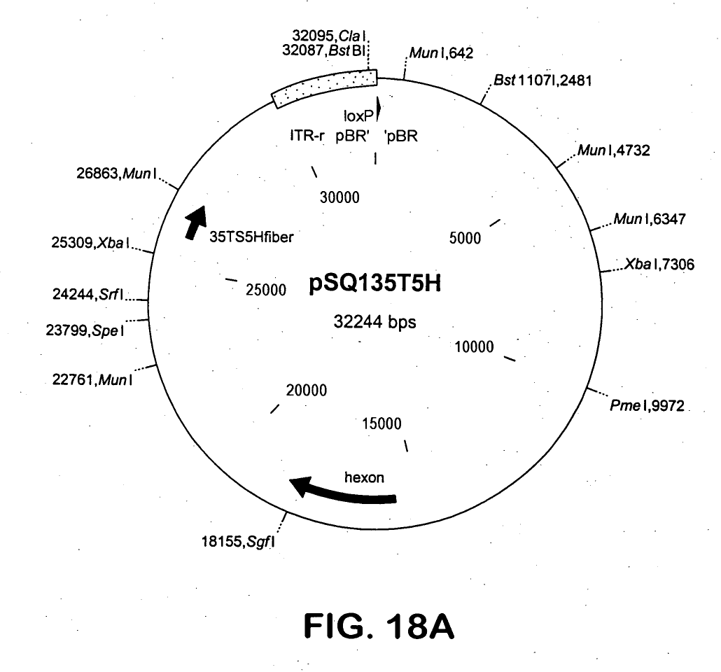

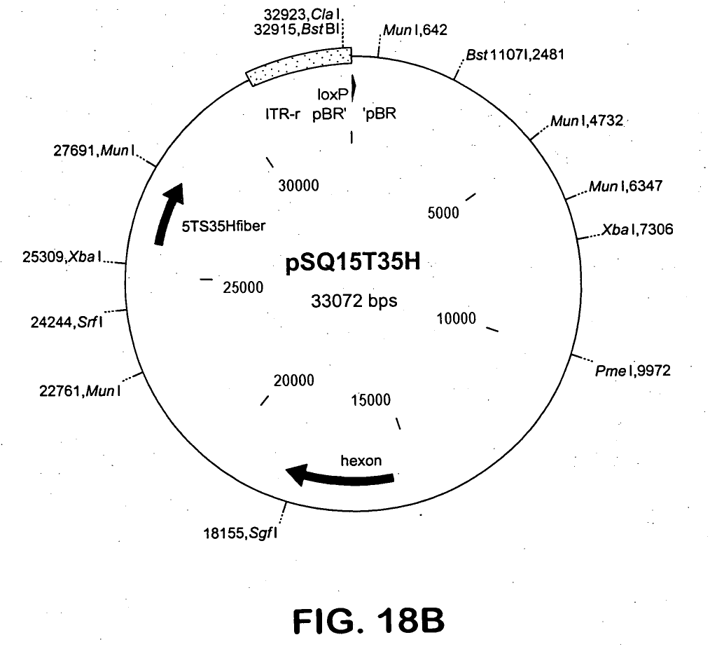

For the 35TS5H and 5TS35H chimeras, the fiber gene was transferred from the pFBshuttle(EcoRI) backbone into pSQ1 as described above for the vector containing the Ad35 fiber. The resulting plasmids were called pSQ135T5H (FIG. 18A) and pSQ15T35H (FIG. 18B). In addition, adenoviral vectors were generated using the co-transfection strategy described above.

Construction of Ad5 vectors containing the Ad35 fiber with a cRGD targeting peptide in the HI loop of the 35F fiber knob: To incorporate the cRGD targeting peptide into the Ad35 fiber HI loop, the P7 and P8 oligonucleotide primers encoding the ten amino acid sequence HCDCRGDCFC (SEQ ID NO. 78) were synthesized. The pFBshuttleAd35 plasmid containing the full length Ad35 fiber cDNA was used as the template in the PCR reaction with the P1 and P7 primer pair or with the P4 and P8 primer pair in order to generate the 5′ and 3′ PCR fragments. A second PCR was then carried out with the end primers P1 and P4 to join the two fragments together. The resulting PCR fragment was digested with XbaI and MunI and was cloned into pFBshuttle (EcoRI) to create the fiber shuttle plasmid pFBshuttleAd35cRGD. The modified Ad35 fiber gene was transferred into pSQ1 using the EcoRI cloning strategy described above to generate pSQ1Ad35FcRGD (FIG. 17B). Adenoviral vector was generated using the co-transfection strategy described above.

### Example 10

### In Vitro Evaluation of Adenoviral Vectors Containing 35F and Derivatives Thereof

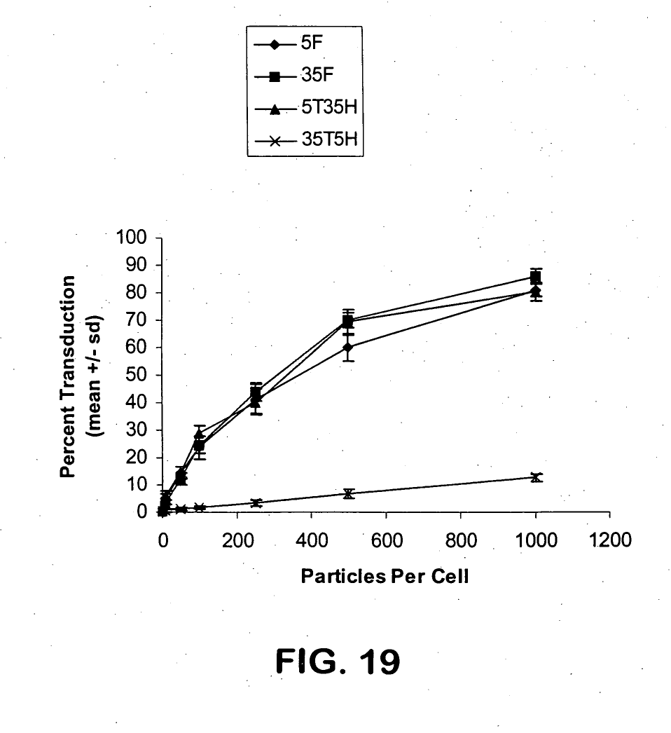

The transduction efficiencies of adenoviral vectors containing the 35F or derivatives thereof were evaluated on A549 cells. The transduction efficiencies were compared to that of Av1nBg, an adenoviral vector containing the 5F fiber. The day prior to infection, cells were seeded into 24-well plates at a density of approximately 1×10⁵ cells per well. Immediately prior to infection, the exact number of cells per well was determined by counting a representative well of cells. Each of the vectors, Av1nBg, Av1nBg35F, Av1nBg5T35H and Av1nBg35T5H were used to transduce A549 cells from 0 up to 1,000 particle per cell (PPC) ratios. The cell monolayers were stained with X-gal 24 hours after infection and the percentage of cells expressing β-galactosidase was determined by microscopic observation and counting of cells. Transductions were done in triplicate and three random fields in each well were counted, for a total of nine fields per vector. The results (FIG. 19) showed similar transduction efficiencies on A549 cells using the Av1nBg35F and Av1nBg5T35H vectors compared to Av1nBg. The Av1nBg35T5H showed much lower transduction efficiencies on A549 cells compared to Av1nBg as a result of the Ad35 shaft domain. The Ad35 shaft domain does not contain a HSP binding motif and the Av1 nBg35T5H vector behaves similarly to the Av1 nBgS* vector in vitro and in vivo. These studies also demonstrate that vectors containing fiber proteins without an HSP binding site are fully viable.

### Example 11

### In Vivo Evaluation of Adenoviral Vectors Containing 35F And Derivatives Thereof

The objective of this study was to evaluate the in vivo biodistribution of adenoviral vectors containing 35F fibers and derivatives thereof to determine whether vectors containing these fibers ablate liver transduction due to their shaft regions. A positive control cohort received Av1nBg and a negative control group received HBSS. Cohorts of five C57BL/6 mice received each vector via tail vein injection at a dose of 1×10¹³ particles per kg. The animals were sacrificed approximately 72 hours after vector administration by carbon dioxide asphyxiation. Liver, heart, lung, spleen, and kidney were collected from each animal. The median lobe of the liver was placed in neutral buffered formalin to preserve the sample for B-galactosidase immunohistochemistry. In addition, tissue from each organ was frozen to preserve it for hexon PCR analysis to determine vector content. A separate sample of liver from each mouse was frozen to preserve it for a chemiluminescent B-galactosidase activity assay. β-galactosidase immunohistochemistry, hexon real-time PCR and the chemiluminescent, β-galactosidase activity assay were carried out as described in example 3.

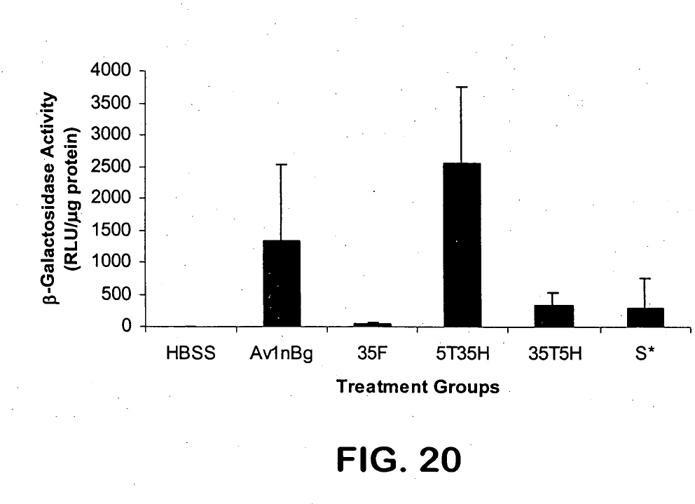

The results of the β-galactosidase activity assay showed a dramatic reduction in liver transduction by vectors containing the Ad35 fiber or the 35T5H derivative (FIG. 20) with an approximately 4- to 24-fold reduction in β-galactosidase activity in the liver compared to the control vector Av1nBg. These data demonstrate that shaft domains without HSP binding sites can effectively ablate hepatic in vivo gene transfer. In particular, HSP is the major entry mechanism for liver in vivo. CAR binding is a minor entry pathway.

### Example 12

### Construction of Ad5 Vectors Containing the Ad Serotype 41 Short Fiber and Derivatives Thereof

The human adenovirus serotype 41 contains two different fibers on its capsid, encoded by two adjacent genes. One fiber has a molecular weight of 60 kDa and is approximately 315A in length and is termed the long fiber. The other fiber has a molecular weight of 40 kDa and is approximately 250+ in length and is termed the short fiber. The Ad41 short fiber does not bind CAR and does not possess the heparin binding domain (KKTK) in its shaft. Therefore, this fiber provides a useful platform for adenoviral vector targeting.

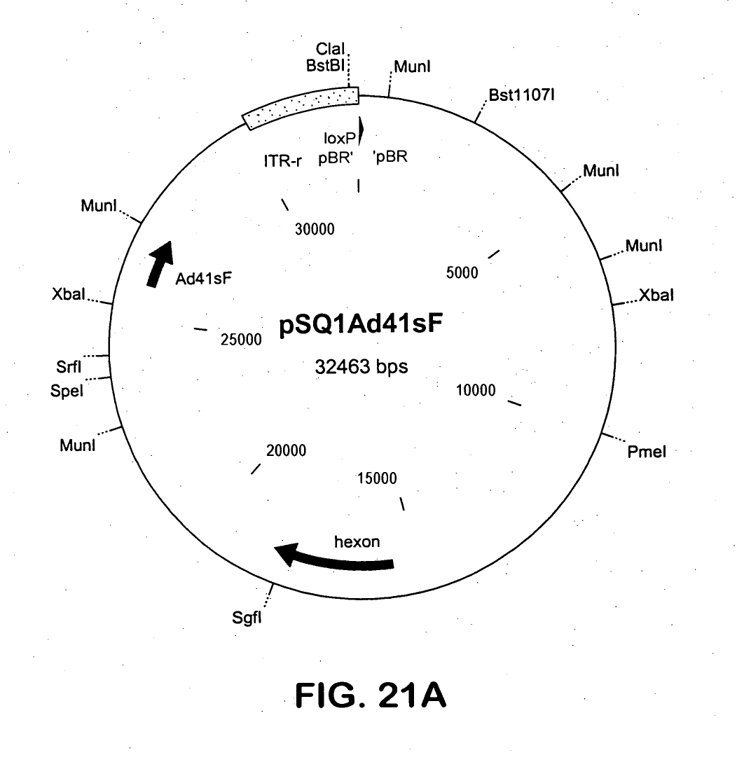

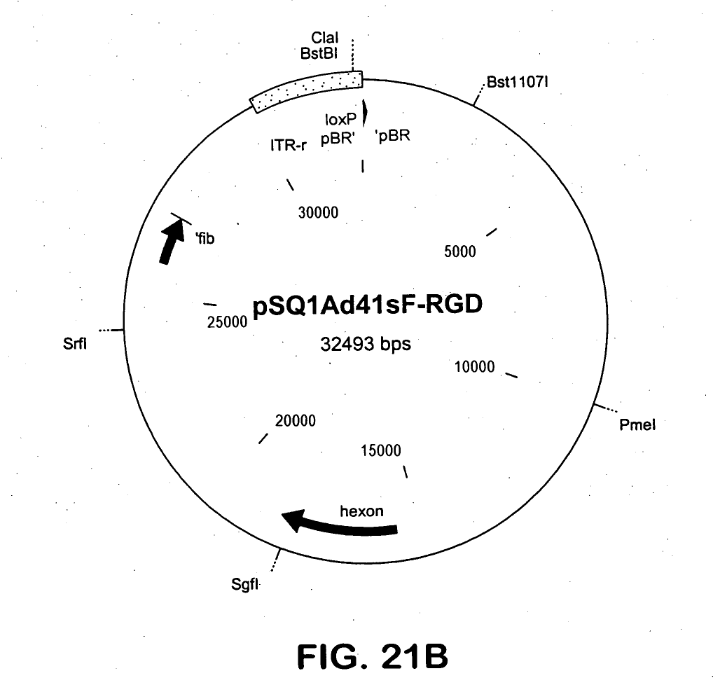

Construction of adenoviral vectors based on Ad5 but containing the Ad41 short fiber: A PCR SOEing strategy was used to generate a vector based on the Ad5 genome but containing the Ad41 short (Ad41s) fiber. First, PCR was used to amplify the region of pSQ1 between the XbaI site at bp 25,309 and the start of the fiber gene. The primer pair used for the PCR were P-0005/U and P-0010L. The DNA sequence of P-0005/U was as follows: 5° C. TCT AGA AAT GGA CGG AAT TAT TAC AG 3′ (SEQ ID NO. 65). The sequence corresponds to bp 25,308 through 25,334 of pSQ1 and overlaps the XbaI site in that region. The DNA sequence of P-0010L was as follows: 5′ TTC TTT TCA T CTG CAA CAA CAT GAA GAT AGT G 3′ (SEQ ID NO. 79). It contains a 5′ extension corresponding to the first 10 bp of the Ad41s fiber gene. The remainder of the primer anneals to pSQ1 immediately 5′ of the ATG start codon of the fiber gene. The PCR product was the expected size (583 bp). A second PCR was used to amplify the Ad41s fiber using the plasmid pDV60Ad41sF as a template. The primers used were P-0011/U and P-0012/L. The DNA sequence of P-0011/U was as follows: 5′ GT TGT TGC AG ATG AAA AGA ACC AGA ATT GAA G 3′ (SEQ ID NO. 80). It contains a 10 bp 5′ extension corresponding to the DNA sequence immediately 5′ of the ATG start codon of the fiber gene in pSQ1. The remainder of the primer anneals to the beginning of the Ad41s fiber gene in pDV60Ad41sF. The DNA sequence of P-0012/L was as follows: 5′ TG CAA TTG AAA AAT AAA CAC GTT GAA ACA TAA CAC AAA CGA TTC TTT ATT C TTC AGT TAT GTA GCA AAA TAC A 3′ (SEQ ID NO. 81). It contains a 51 bp 5′ extension corresponding to the sequence in pSQ1 from the last codon of the fiber gene through the MunI site 40 bp downstream of the fiber gene. The remainder of the primer anneals to the 3′ end of the Ad41s fiber gene in pDV60Ad41sF. The PCR product was the expected size (1219 bp). The two PCR products were joined by PCR SOEing using primers P-0005/U and P-0009/L. The DNA sequence of P-0009/L was described above. The PCR SOEing reaction yielded the expected 1782 bp product. The product was cloned into pCR4blunt-TOPO to yield pCR4blunt-TOPOAd41sF. Next, pCR4blunt-TOPOAd41sF was digested with XbaI and MunI and the 1773 bp fragment containing the Ad41s fiber gene was gel purified. This fragment was ligated with the 6477 bp XbaI to MunI fragment of pFBshuttle(EcoRI) to generate pFBshuttleAd41sF. The Ad41s fiber gene was transferred into the pSQ1 backbone as follows. First, pFBshuttleAd41sF was linearized using EcoRI and this fragment was ligated with the 24,213 bp EcoRI fragment of pSQ1 to generate pSQ1Ad41sF (FIG. 21A). Adenoviral vector containing the Ad41s fiber was generated using the co-transfection strategy described above.

Construction of Ad5 adenoviral vectors containing the Ad41 short fiber with a cRGD targeting ligand in the Hi loop: A PCR SOEing strategy was used to generate a construct containing the Ad41s fiber with cRGD in the HI loop. The plasmid pFBshuttleAd41sF was used as a template for the PCR amplifications. First, a 1782 bp fragment was amplified using primers 5FF and 41sRGDR. The primer 5FF was described above. It anneals to pFBshuttleAd41sF at the XbaI site upstream of the fiber gene. The DNA sequence of the primer 41sRGDR was as follows: 5′ AGT ACA AAA ACA ATC ACC ACG ACA ATC ACA GTT TAT CTC GTT GTA GAC GAC ACT GA 3′ SEQ ID NO. 82). It contains a 30 bp 5′ extension that encodes the cRGD targeting ligand. The remainder of the primer anneals to pFBshuttleAd41sF from bp 2878 through 2903. A second PCR amplified a 277 bp region of pFBshuttleAd41sF using primers 3FR and 41sRGDF. The primer 3FR was described previously. It anneals to pFBshuttleAd41 sF at the MunI site downstream of the fiber gene. The DNA sequence of 41 sRGDF was as follows: 5′ TGT GAT TGT CGT GGT GAT TGT TTT TGT ACT AGT GGG TAT GCT TTT ACT TTT 3′ (SEQ ID NO. 83). It contains a 30 bp 5′ extension that encodes the cRGD targeting ligand and is complementary to the extension on 41sRGDR. The remainder of the primer anneals to pFBshuttleAd41sF from bp 2904 through 2924. The two PCR products were joined by PCR SOEing to generate a 2059 bp fragment using primers 5FF and 3FR. The product was digested with XbaI and MunI and the 1803 bp DNA fragment was gel purified. The fragment was ligated with the 6477 bp fragment resulting from digestion of pFBshuttle(EcoRI) with XbaI and MunI. The resulting plasmid was termed pFBshuttleAd41sRGD. This plasmid was linearized by EcoRI digestion and ligated with the 24,213 bp EcoRI fragment of pSQ1 to generate pSQ1Ad41sRGD (FIG. 21B).

### Example 13

### In Vivo Evaluation of Ad5 Vectors Containing the Ad41 Short Fiber and Derivatives Thereof

This example evaluates the in vivo biodistribution of adenoviral vectors containing 41sF fibers and derivatives thereof to determine whether vectors containing the these fibers ablate liver transduction due to modified shaft regions. A positive control cohort received Av3nBg (see, Gorziglia et al. (1996) J. Virology 70:4173-4178) or Ad5.βGal.ΔF/5F, and a negative control group received HBSS. Ad5.βGal.ΔF/5F is a derivative of the fiberless vector Ad5.βgal.ΔF (ATCC accession number VR2636) modified to express AD5 fiber (see, e.g., International PCT application No. WO 01/83729).

The Ad5.βGal.ΔF vector was pseudotyped with the Ad41sF fiber protein and injected in vivo. Cohorts of five C57BL/6 mice received each vector via tail vein injection at a dose of 1×10¹³ particles per kg. The animals were sacrificed approximately 72 hours after vector administration by carbon dioxide asphyxiation. Liver, heart, lung, spleen, and kidney were collected from each animal. The median lobe of the liver was placed in neutral buffered formalin to preserve the sample for β-galactosidase immunohistochemistry. In addition, tissue from each organ was frozen to preserve it for hexon PCR analysis to determine vector content. A separate sample of liver from each mouse was frozen to preserve it for a chemiluminescent β-galactosidase activity assay. β-galactosidase immunohistochemistry, hexon real-time PCR and the chemiluminescent β-galactosidase activity assay was carried out as described in Example 3.

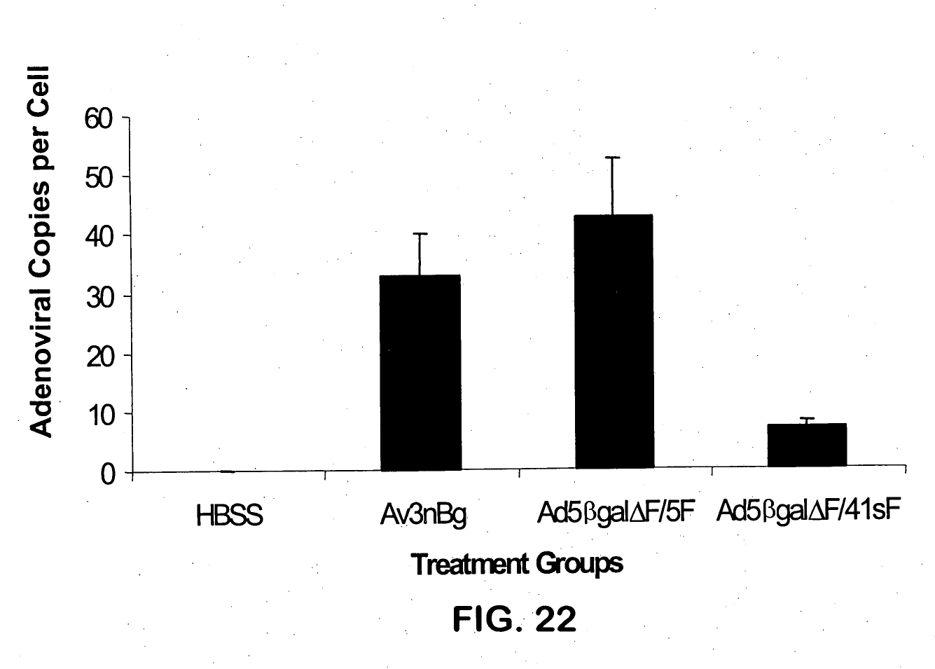

The results of the hexon DNA analysis showed a dramatic reduction in liver transduction by vectors containing the Ad41sF fiber (FIG. 22) with an approximately a 5-fold reduction in liver adenoviral DNA content compared to either control vector.

In the above examples, several novel adenoviral vectors were generated containing various fiber modifications designed to ablate the normal tropism of the vector (see Table 3). Vectors were generated in which the heparan sulfate binding domain in the fiber shaft was replaced by amino acid substitutions. This mutation, termed HSP, was also combined with the KO1 mutation (fiber knob AB loop mutation that ablates CAR binding), and the PD1 mutation (penton mutation that eliminates RGD/integrin interaction). In addition, a vector containing all three mutations (HSP, KO1, PD1) was generated. All vectors containing the HSP mutation, either alone or combined with other capsid modifications, showed dramatically reduced transduction efficiencies on A549 and HeLa cells. Furthermore, the same vectors showed dramatically reduced transduction of the liver following systemic delivery to mice. As an alternative strategy to ablate the normal tropism of Ad5-based vectors, the Ad5 fiber was replaced by a fiber from a different adenovirus serotype which does not bind CAR and does not contain the heparan binding domain in the shaft. Thus, vectors were generated containing the Ad35 fiber and the Ad41 short fiber. Versions of these two vectors containing a cRGD targeting ligand in the HI loop of the fiber were also produced. Additionally, vectors containing chimeric fibers were generated. A vector containing the Ad35 fiber tail and shaft regions fused to the Ad5 fiber knob domain as well as a vector containing the Ad5 fiber tail and shaft fused to the Ad35 fiber knob domain were constructed. Vectors containing either the entire Ad35 or Ad41 short fiber showed a significant reduction in liver transduction following delivery to mice via the tail vein. The observation of reduced liver transduction using vectors containing either an HSP mutation, the Ad35 fiber, or the Ad41 short fiber indicates the feasibility of detargeting adenoviral vectors in vivo. In vitro data with the Ad35 fiber or the Ad41 short fiber with cRGD (see Example 14) indicate that the virus is completely viable, that is, it is not damaged by the absence of an HSP binding site and is retargetable. Taken together these data suggest that these vectors provide a suitable platform for retargeting strategies.

TABLE 3
Description Of Recombinant Adenoviral Vectors Used To Demonstrate
That Shaft Modifications Influence Tropism In Vivo Vector

| Vector | Description |
|---|---|
| Av1nBg | An E1 and E3-deleted adenoviral |
| | vector encoding a nuclear localizing |
| | β-galactosidase |

Ad5 Fiber derivatives:

| | |
|---|---|
| Av1nBgFKO1 | The same as Av1nBg but containing |
| | the KO1 AB loop mutation in the |
| | fiber gene |
| Av1nBgPD1 | The same as Av1nBg but containing |
| | the penton PD1 mutation that deletes |
| | the integrin binding, RGD tripeptide |
| Av1nBgS* | The same as Av1nBg but containing |
| | the 4 amino acid substitution in the |
| | shaft referred to as S* that modifies |
| | the HSP binding motif |
| Av1nBgFKO1S* | The same as Av1nBg but containing |
| | the fiber KO1 and S* mutations |
| | combined |
| Av1nBgS*PD1 | The same as Av1nBg but containing |
| | the fiber S* and penton PD1 |
| | mutations combined |
| Av1nBgFKO1S*PD1 | The same as Av1nBg but containing |
| | the fiber KO1, S* and penton PD1 |
| | mutations combined |

Ad35 fiber derivatives:

| | |
|---|---|
| Av1nBg35F | The same as Av1nBg but containing |
| | the full length Ad35 fiber cDNA |
| Av1nBg5T35H | The same as Av1nBg but contain- |
| | ing the 5T35H chimeric fiber |
| Av1nBg35T5H | The same as Av1nBg but containing |
| | the 35T5H chimeric fiber |
| Av1nBg35FRGD | The same as Av1nBg but containing |
| | the full length Ad35 fiber cDNA |
| | with a cRGD ligand in the HI loop |
| | of the Ad35 fiber |

Ad41sF fiber derivatives:

| | |
|---|---|
| Av1nBg41sF | The same as Av1nBg but containing |
| | the full length Ad41 short fiber |
| | cDNA |
| Av1nBg41sFRGD | The same as Av1nBg but containing |
| | the full length Ad41 short fiber |
| | cDNA with a cRGD ligand in the HI |
| | loop of the Ad41 short fiber |

### Example 14

### In Vitro Evaluation of Adenoviral Vectors Containing the Ad41sF with a cRGD Ligand in the Hi Loop

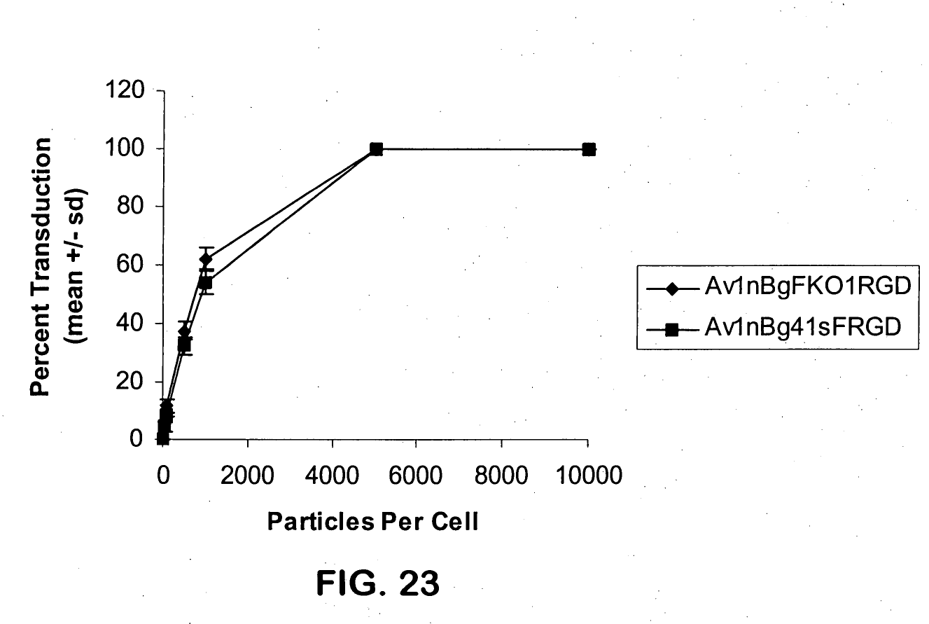

The transduction efficiencies of adenoviral vectors containing the Ad41sF fiber with the cRGD ligand in the HI loop were evaluated on A549 cells. The transduction efficiencies were compared to that of Av1nBg, an adenoviral vector containing wild type fiber or Av1nBgFKO1RGD, an adenoviral vector containing the KO1 mutation in combination with the cRGD ligand in the HI loop. The day prior to infection, cells were seeded into 24-well plates at a density of approximately 1×10⁵ cells per well. Immediately prior to infection, the exact number of cells per well was determined by counting a representative well of cells. Each of the vectors, Av1nBg, Av1nBgFKO1RGD, and Av1nBg41sFRGD were used to transduce A549 cells at a particle to cell ratios of 0 up to 10,000. The cell monolayers were stained with X-gal 24 hours after infection and the percentage of cells expressing β-galactosidase was determined by microscopic observation and counting of cells. Transductions were done in triplicate and three random fields in each well were counted, for a total of nine fields per vector. The results (FIG. 23) show that the Av1nBg41sFRGD vector transduced cells to an equivalent level as Av1 nBgFKO1 RGD at all vector doses examined. Neither FKO1 or Ad41sF can bind CAR. The Ad41sF does not normally interact with CAR and additionally does not contain the HSP binding motif within the shaft domain. These data show that targeting peptides inserted into the loop regions of the fiber knob of KO1 and Ad41sF allows for transduction of target cells via the targeted receptor. Surprisingly, HSP, not CAR and integrins, is the major entry route in vivo and ablation of HSP binding permits targeting of adenoviral vectors.

### Example 15

### Effect of the Shaft Modification on the Biodistribution of Adenoviral Vectors In Vivo

The influence of fiber and penton modifications on the in vivo biodistribution of adenoviral vectors containing fiber head, shaft and penton mutations was examined. Vectors containing the HSP mutation combined with KO1, or PD1, or a combination of all three mutations were evaluated as well as vectors containing the KO1 mutation alone and the PD1 mutation alone. The indicated adenoviral vectors were systemically administered to C57BL6 mice as described above. A positive control cohort received Av1nBg and a negative control group received HBSS. Cohorts of five C57BL/6 mice received each vector via tail vein injection at a dose of 1×10¹³ particles per kg. The animals were sacrificed approximately 72 hours after vector administration by carbon dioxide asphyxiation. Liver, heart, lung, spleen, and kidney were collected from each animal. Tissue from each organ was frozen to preserve it for real time PCR analysis to determine adenoviral hexon DNA content. A separate sample of liver from each mouse was frozen to preserve it for a chemiluminescent β-galactosidase activity assay. Hexon real-time PCR and the chemiluminescent β-galactosidase activity assay was carried out as described in Example 3.

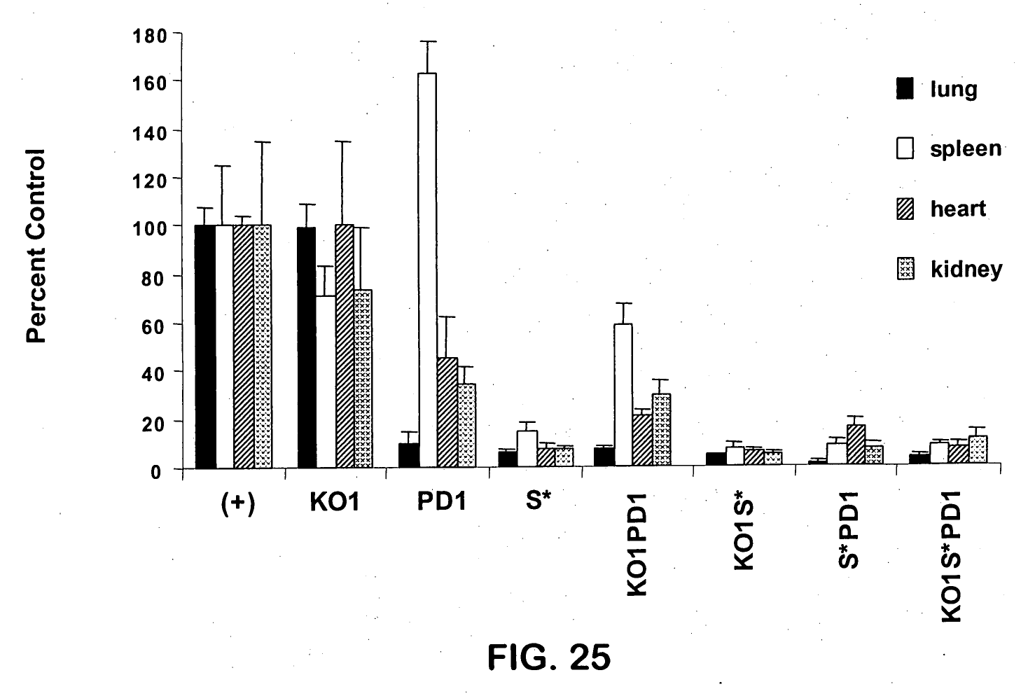

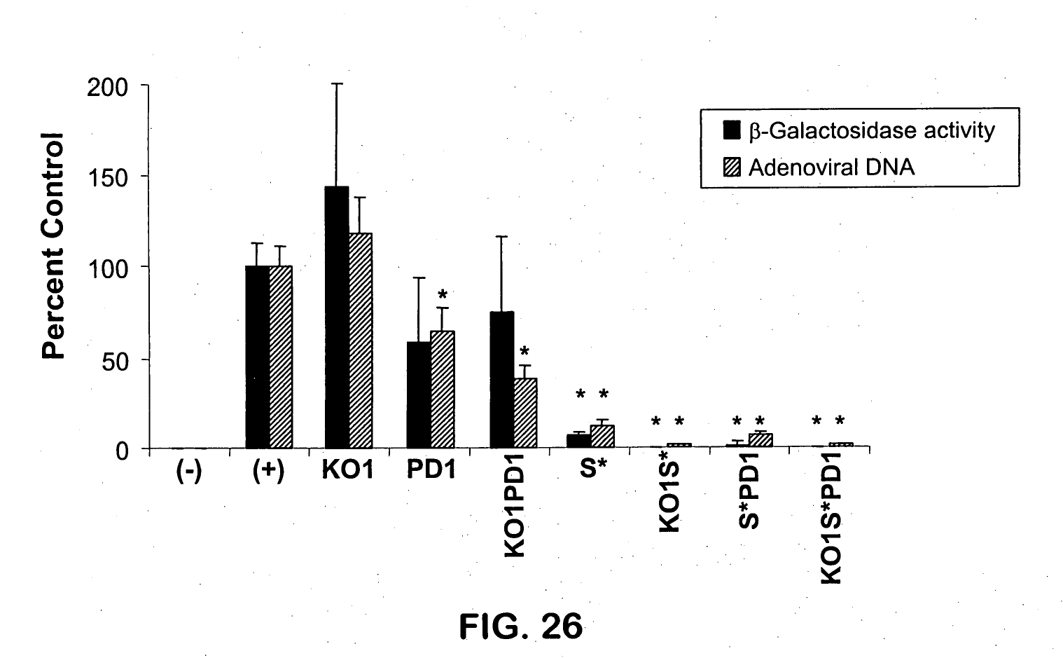

The results derived from the liver are described in Example 6 (FIGS. 14A and B) and also shown in FIG. 26 with results presented as percent control of Av1nBg. The effect of the S* shaft modification on the biodistribution of adenovirus to the other organs is shown in FIG. 25. The average adenoviral DNA content was determined as adenoviral genomic copies per cell and expressed as a percentage of the Av1nBg (+) control value. The average percent control value+standard deviation is shown (n=5 per group) for each tissue examined (FIG. 25).

Systemic delivery of Ad5 based vectors with wild-type fiber results in a preferential accumulation of vector DNA in the liver with 64 copies per cell with significantly less DNA found in the other organs with 1.32 copies per cell found in lung, 2.18 copies per cell in spleen, 0.47 copies per cell found in heart, and 0.72 copies per cell in the kidney. All differences found with PD1, S*, KO1PD1, KO1S*, S*PD1, and KO1S*PD1 were significantly different than the Av1nBg (+) control using a unpaired, t-test analysis, P value (0.024. When expressed as a percent of the Av1nBg control values, the influence of each mutation, individually or in combination, becomes apparent. The S* mutation dramatically reduced gene transfer to all four organs, whereas, the KO1 mutation did not. Thus, the importance of the shaft for transduction in vivo extends to organs besides the liver. Finally, gene transfer to the lung, heart, and kidney was diminished with PD1 suggesting a role for integrin binding in vector entry in these organs.

### Example 16

### Retargeting the S*, Shaft Modification and the 41sF Fiber In Vivo

Vectors containing the HSP mutation have been shown to effectively detarget adenoviral vectors in vivo (see examples 6 and 15). The objective of this study was to evaluate the ability to retarget vectors containing the S* modification or the Ad41sF to tumors in vivo. A cRGD peptide was genetically incorporated into the fiber HI loop and evaluated in vitro (Examples 8 and 14). These same vectors were then evaluated in vivo in tumor-bearing mice. Athymic nu/nu female mice were injected with 8×10⁶ A549 cells on the right hind flank. When tumors reached approximately 100 mm3 in size, they were randomized into treatment groups. Cohorts of 6 mice received each vector via tail vein injection at a dose of 1×10¹³ particles per kg. The animals were sacrificed approximately 72 hours after vector administration by carbon dioxide asphyxiation. Tumor, liver, heart, lung, spleen, and kidney were collected from each animal. Tissue from each organ was frozen to preserve it for real time PCR analysis to determine adenoviral hexon DNA content. Hexon real-time PCR was carried out as described in example 3. A separate sample of liver from each mouse was frozen to preserve it for a chemiluminescent β-galactosidase activity assay. Hexon real-time PCR and the chemiluminescent β-galactosidase activity assay was carried out as described in example 3.

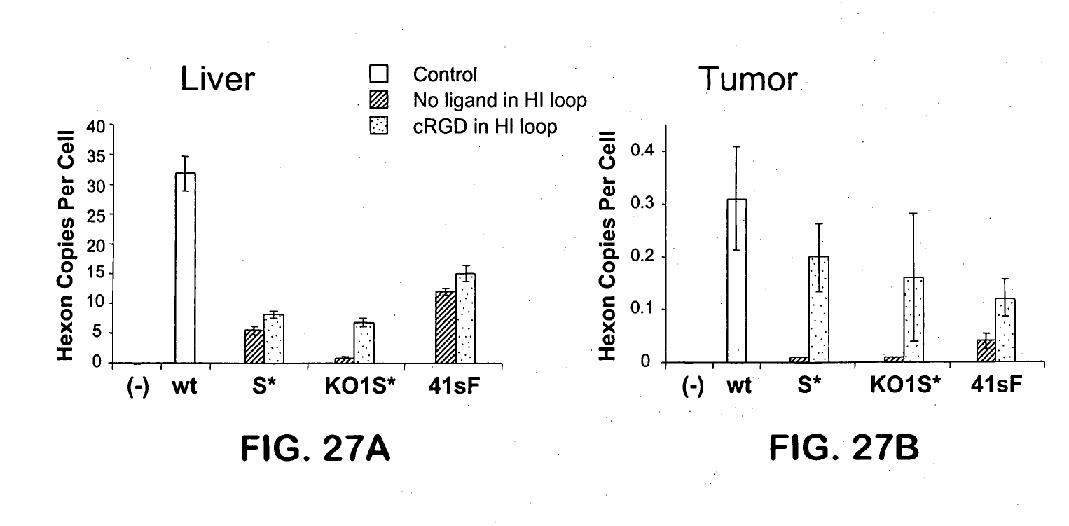

The adenoviral vector biodistribution to the liver and tumor for each treatment group is shown in FIG. 27. Vectors containing the S*, KO1S*, and 41sF fibers effectively detargeted the liver and tumor resulting in a significant reduction in the amount of adenoviral DNA found in each tissue in comparison to the Av1nBg control. Vectors containing the cRGD targeting ligand restored transduction of the tumors to levels comparable to that achieved with the untargeted vector.

These data demonstrate successful liver detargeting accompanied with tumor retargeting. The extent of tumor retargeting is relates to the affinity and type of ligand that is used. These data demonstrate the successful development of a targeted, systemically deliverable adenoviral vector that will target tumors in vivo.

### Example 17

### Scale-Up Method for the Propagation of Detargeted Adenoviral Vectors

The growth and propagation of doubly or triply ablated adenoviral vectors requires novel scale up technologies. These detargeted vectors require alternative cellular entry strategies to allow for the efficient growth and generation of high titer preparations. A strategy for vector growth that is generally applicable to all detargeted adenoviral vectors, that does not require the development of new cell lines, and that aslo can be used for generating targeted vectors is provided herein.

Three recombinant adenoviral vectors were prepared that contain single mutations in the fiber or penton or both mutations combined into one vector. These vectors are designated Av3nBgFKO1, Av1nBgPD1, and Av1nBgFKO1PD1, respectively. The construction of these vectors is described above and a general description of each vector can be found in Table 1 above.

Scale-up of detargeted adenoviral vectors: A polycation, specifically hexadimethrine bromide was obtained from Sigma Chemical Co (St. Louis, Mo.), Catalog No. 52495, and was maintained in the medium at 4 μg/ml during the course of transfections and infections. To illustrate the affects of hexadimethrine bromide on the yield of detargeted adenoviral vectors the following experiment was carried out. Seven plates of AE1-2a adenoviral producer cells (Gorziglia et al. (1996) J. Virology 70:4173-4178) were transduced with 10 particles per cells of each of the indicated vectors (See Table 4). Each vector was incubated with medium (Richters with 2% HI-FBS) containing hexadimethrine bromide at 4 μg/ml for 30 min at room temperature prior to infection. The infection was carried out for 2 hrs. Complete medium containing hexadimethrine bromide at 4 μg/ml was added to each plate. Final concentration of hexadimethrine bromide in all of these experiments was maintained at 4 μg/ml. The titers were determined spectrophotometrically using the conversion of 10D at A260 nm per 1×10¹² particles (Mittereder et al. (1996) J Virology 70:7498-7509). The total particle yield was then normalized for the number of plates used for transduction.

The inclusion of hexadimethrine bromide in the medium during the course of infection allows for the efficient propagation of detargeted adenoviral vectors containing fiber and penton mutations either alone or in combination. The effect of hexadimethrine bromide on vector yields is shown in Table 4. A 35-fold improvement in the yield of Av3nBgFKO1 was found when hexadimethrine bromide was included in the culture medium and resulted in increasing the vector yield from 1.3×10¹⁰ up to 4.6×10¹¹ vector particle per plate. Hexadimethrine bromide has a minimal effect on the yield of the Av1nBgPD1 adenoviral vector containing the penton, PD1 mutation with only a 1.2 fold improvement. The greatest effect using hexadimethrine bromide was found on the propagation of the doubly ablated adenoviral vector, Av1nBgFK01PD1 with increases in vector yield from barely detectable levels up to 4.53×10¹⁰ vector particles per plate. These data demonstrate that use of nonspecific entry mechanisms allows for the efficient scale-up of detargeted adenoviral vectors.

TABLE 4
Efficient Scale-Up Of Detargeted Adenoviral
Vectors Using hexadimethrine bromide

| | Vector Yield (particles/plate) | | |
|---|---|---|---|
| | (−) hexadi- | (+) hexadi- | |
| | methrine | methrine | Fold |
| Vector | bromide | bromide | Improvement |
| Av1nBg | 3.89 × 10¹¹ | 5.72 × 10¹¹ | 1.47 |
| Av3nBg | 8.58 × 10¹⁰ | 2.38 × 10¹¹ | 2.77 |
| Av3nBgFKO1 | 1.30 × 10¹⁰ | 4.60 × 10¹¹ | 35.4 |
| Av1nBgPD1 | 1.95 × 10¹¹ | 2.40 × 10¹¹ | 1.23 |
| Av1nBgFKO1PD1 | TLTC* | 4.53 × 10¹⁰ | ^{†} |

*TLTC: Too low to count, a faint virus band was collected and the particle concentration was too dilute for titer determination.
^{†}Significant improvement

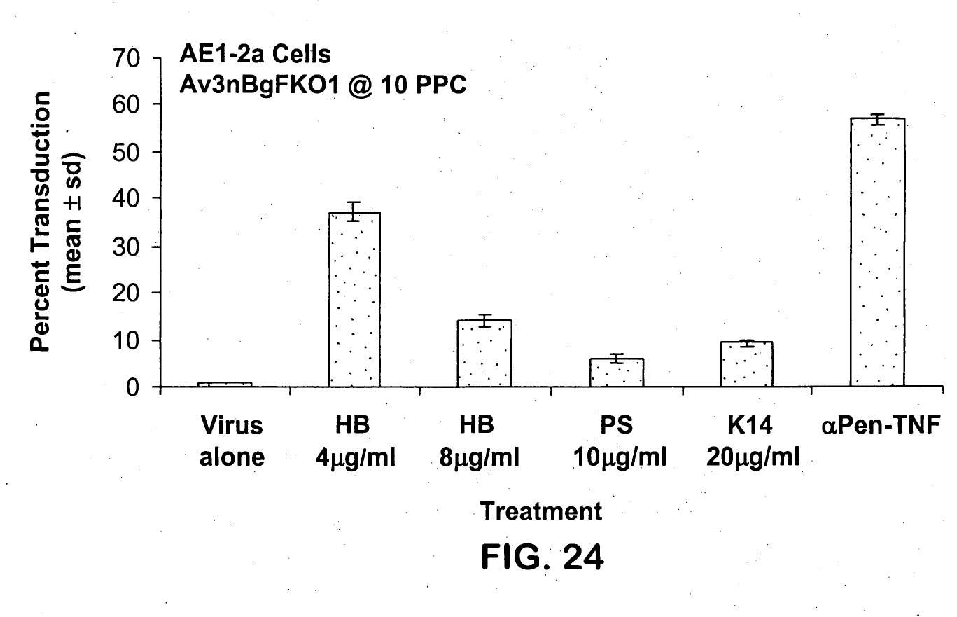

The use of alternative polycations including protamine sulfate and poly-lysine as well as bifunctional proteins such as the anti-penton:TNFα fusion protein was investigated. FIG. 24 show results that demonstrate all the reagents tested had some effect on enhancing transduction of the Av3nBgFKO1 vector. All of these compounds, when maintained in the medium during infection, enhanced transduction of the Av3nBgFKO1 detargeted adenoviral vector.

Bifunctional reagents: The use of bifunctional reagents for the propagation of detargeted adenoviral vectors was examined using the anti-penton:TNFα fusion protein. This particular reagent is a fusion protein between an antibody against Ad5 penton and the TNFα protein that is produced using stably transfected insect cells. This reagent will bind specifically to the adenoviral capsid via penton base and allow for binding to cell surface TNF receptors. The use of this reagent for the propagation of detargeted vectors is illustrated in Table 5 using Av3nBgFKO1 (also shown in FIG. 24). Monolayers of S8 cells were infected with 10 or 100 particles per cell of Av3nBgFKO1 or a control vector in the presence or absence of 1 μg/ml of the anti-penton:TNFα fusion protein. The monolayers were visually inspected over time for vector spread as indicated by the extent of cytopathic effect (CPE). The percentage of CPE at each time point is shown. The use of this bifunctional reagent clearly enhances the spread of the Av3nBgFKO1 vector throughout the monolayer.

TABLE 5
Efficient Scale-Up Of Detargeted Adenoviral Vectors
Using Bifunctional Reagents: Anti-Penton: TNFα

| | | | |
|---|---|---|---|
| 10 ppc − | 10 ppc + | 100 ppc − | 100 ppc + |
| anti-penton | anti-penton | anti-penton | anti-penton |
| TNF | TNF | TNF | TNF |

Percentage of CPE

Ad5Luc1

| | | | | | |
|---|---|---|---|---|---|
| 24 | h | 0% | 0% | 0% | 0% |
| 48 | h | 20-30% | 20-30% | 90-100% | 90-100% |
| 72 | h | 60-70% | 80-90% | 100% | 100% |
| 120 | h | 100% | 100% | 100% | 100% |

Av3nBgKO1 24 hrs

| | | | | | |
|---|---|---|---|---|---|
| 24 | h | 0% | 0% | 0% | 0% |
| 48 | h | 0% | 10-20% | 0% | 90-100% |
| 72 | h | 5% | 60-70% | 5% | 100% |
| 120 | h | 40-50% | 100% | 100% | 100% |

### Example 18

This Example and the following Example describe construction of adenoviral Ad5 particles that express heterologous fibers. The fibers are modified at the N-terminus to increase incorporation into the Ad5 particle. The N-terminus, typically, the first at least 16 or 17 amino acids is modified so that the sequence resembles the Ad5 terminus.

### Expression of Fiber Proteins from Subgroups B, C and D of Human Adenovirus

Constructs for expression of fibers from several adenoviral serotypes including, Types 16, 30, and 35 (subgroup B), and Types 19p and 37 (subgroup D), were generated.

### Construction of Ad37, Ad19p and Ad30 Fiber Expression Plasmids

The open reading frames (ORFs) of Ad37 (SEQ ID NO. 31), Ad19p (SEQ ID NO. 33) or Ad30 (SEQ ID NO. 35) fiber proteins were PCR amplified using the following primers:

Forward primer (L37):

(SEQ ID NO. 84)

TGT CTT GGA TCC AAG ATG AAG CGC GCG CGC CCC AGC
GAA GAT GAC TTC
Reverse primer (37FR):

(SEQ ID NO. 85)

AAA CAC GGC GGC CGC TCT TTC ATT CTT G

Primers L37 and 37FR include BamHI and NotI sites (in bold), respectively, to facilitate subcloning. In addition, primer L37 introduces mutations into the 5′ end of each fiber protein so that the resulting fiber proteins more closely resemble the Ad5 fiber N-terminal sequence for assembly onto Ad5 particles. For Ad37, the native N-terminus and modified N-terminus have the following amino acid sequences:

| | | |
|---|---|---|
| Native Ad37 N-terminus: | MSKRLRVE | (SEQ ID NO. 86) |
| Modified Ad37 N-terminus: | MKRARPSE | (SEQ ID NO. 87) |

The amplified fibers were cloned into the BamHI and NotI sites of plasmid pcDNA3.1zeo(+) (Invitrogen). The Ad5 tripartite leader (TPL) sequence from plasmid pDV55 (see EXAMPLE 20 and SEQ ID NO. 88), which is flanked by BamHI sites (SEQ ID NO. 88), was then subcloned into the BamHI site of each fiber expression plasmid to generate plasmids pDV121 (Ad37), pDV145 (Ad19p) and pDV164 (Ad30). Construction of plasmid pDV55 is set forth in EXAMPLE 20 (see also, copending U.S. application Ser. No. 09/482,682, also filed as International PCT application No. PCT/US00/00265; and in U.S. application Ser. No. 09/562,934, also filed as International PCT application No. PCT/EP01/04863. The combination of the CMV promoter present in pcDNA3.1zeo(+) and the addition of the TPL sequence from pDV55 (SEQ ID NO. 88) provides for high-level expression of viral proteins.

Construction of Ad16 and Ad35 Fiber Expression Plasmids

Ad16 and Ad35 fiber expression plasmids were generated in a similar manner with the following modifications. To PCR amplify Ad16 (SEQ ID NO. 37) and Ad35 (SEQ ID NO. 39) fiber, the forward primer was designed to incorporate an NdeI site (in bold), which is present at nucleotide 48 of Ad5 fiber (SEQ ID NO. 1), but absent in Ad16 and Ad35 fiber sequences. The reverse primers contained a NotI site (in bold):

Ad16/Ad35 forward primer (F16 5′):

(SEQ ID NO. 89)

CCG GTC TAG CCA TAT GAA GATG
Ad16 reverse primer (F16 3′):

(SEQ ID NO. 90)

TGG TGC GGC CGC TCA GTC ATC TTC TCTG
Ad35 reverse primer (F35 3′):

(SEQ ID NO. 91)

TGG TGC GGC CGC TTA GTT GTC GTC TTC TGT AAT G

The Ad16 and Ad35 PCR products were cloned into the NdeI and NotI sites of pcDNA3.1zeo(+), resulting in plasmids pDV147 (Ad16) and pDV165 (Ad35). The NdeI site of pcDNA3.1zeo(+) is within the CMV promoter region of the plasmid, therefore, the resulting plasmids lacked the 3′ portion of the CMV promoter region. In addition, the inserted fiber sequences were lacking the portion of the fiber sequence that is 5′ to the engineered NdeI site. To insert the necessary regulatory sequences and N-terminal fiber sequence, plasmid pDV67 (described in Example 22 and also U.S. application Ser. No. 09/562,934, and is available from the ATCC under accession number PTA-1145) was digested with NdeI to remove a fragment that contains the 3′ portion of the CMV promoter, the complete Ad5 TPL sequence and the 5′ portion of the Ad5 fiber sequence. The NdeI fragment was subcloned into plasmids pDV147 and pDV165 to generate the complete Ad16 and Ad35 expression plasmids, pDV156 and pDV166, respectively. Expression of these constructs results in chimeric fiber proteins containing the 17 N-terminal amino acids from Ad5 fiber (see SEQ ID NO. 2) and the remainder of the fiber sequence from either Ad16 or Ad35. The nucleotide sequences of the chimeric fibers are listed in SEQ ID NO. 41 (Ad5/Ad16) and SEQ ID NO. 43 (Ad5/Ad35).

Expression and Trimerization of Recombinant Ad Fiber Proteins

To verify expression and trimerization of the recombinant proteins, the resulting plasmids were transfected into 293T cells, which are identical to 293 cells except they express an integrated SV40 large T antigen gene. 239 cells are an adenovirus-transformed human embryonic kidney cell line obtained from the ATCC, where they are deposited under Accession Number CRL 1573. 293T cells express CAR and αᵥ integrins. Fiber expression was detected by immunoblotting of cell lysates using the 4D2 monoclonal antibody (Research Diagnostics Inc., Flanders, N.J.), which recognizes an epitope conserved among fibers of different serotypes. To generate stable cell lines, constructs were electroporated into an A549-derived cell line that complements the Ad E1a and E2a functions (Gorziglia et al., J. Virol. 70:4173-4178 (1996)) and stable clones were derived by selection with zeocin. Clones that expressed high levels of the fiber protein were identified by immunoblotting with the 4D2 antibody.

Generation of Adenovirus Particles Pseudotyped with Subgroup B, C or D Ad Fiber Protein

A system for producing Ad vector particles with different or modified fiber proteins, that therefore have altered tropism (pseudotyping) is known (see, e.g., Von Seggern et al., J. Virol. 74:354-362 (2000); Wu et al., Virology 279:78-89 (2001)). Briefly, an E1-deleted Ad vector is modified by further deletion of the fiber gene, such that the virus produces no fiber protein. Growth of the fiber-deleted viruses in packaging cells that express a fiber protein as well as complementing the E1 deletion allows generation of particles with any desired fiber.

Packaging cell lines were generated by stably transfecting expression constructs for the fibers of interest (Von Seggern et al., J. Virol. 74:354-362 (2000)) into an A549-derived E1- and E2a-complementing cell line (Gorziglia et al., J. Virol. 70:4173-4178 (1996)), and clones that expressed the fibers at high levels were selected. The resulting lines complement E1 and fiber deletions, and were used to propagate Ad5.GFP.ΔF, a fiber-deleted Ad5 vector with a GFP transgene in place of the deleted E1 sequences (Von Seggern et al., J. Virol. 74:354-362 (2000)). The particles produced by growth in the various cell lines are identical except for their fiber proteins. Viral particles were isolated by CsCl gradient centrifugation, and assayed for the presence of fiber by immunoblotting using monoclonal Ab 4D2. As a control for equal loading, the blot was re-probed with a polyclonal antibody against the Ad penton base protein. All recombinant fibers were capable of assembly onto Ad5 particles.

### Example 19

### Construction and Propagation of Adenovirus Particles with Genomic Fiber Substitutions

The fiber-deletion system described in Example 18 allows rapid evaluation of fiber proteins for their infectious properties. The resulting particles produced are less infectious than the corresponding first-generation vectors (Von Seggern et al., J. Virol. 74:354-362 (2000)). Therefore, viral backbones with the subgroup B, C or D fibers substituted in place of the Ad5 fiber gene were constructed. To facilitate construction of these vectors, the AdEasy system (see, U.S. Pat. No. 5,922,576; see, also He et al. (1998) Proc. Natl. Acad. Sci. U.S.A. 95:2509-2514; the system is publicly available from the authors and other sources) was modified. This system includes a large plasmid (pAdEasy) that contains most of the Ad5 genome and smaller shuttle plasmids with the left end of the viral genome, including an E1 deletion and polylinker for insertion of transgenes. Recombination between pAdEasy and a shuttle plasmid in E. coli reconstitutes a full-length infectious Ad genome. All plasmids used were derivatives of pAdEasy1 with different fiber proteins substituted in place of the Ad5 fiber.

Construction of pDV153

p5FloxHRF (SEQ ID NO. 92) contains the right end of the Ad5 genome with a PacI site in place of the right ITR. There is a unique (naturally occurring in Ad5) MunI site approximately 30 nucleotides downstream of the fiber stop codon. To facilitate fiber substitutions, this site was moved to lie immediately downstream of the fiber ORF. This preserved the sequence around the fiber gene and its spacing relative to the other adenovirus genes.

The oligos MunITOP (AAT TGT GTT ATG TTT AAA CGT GTT TAT TTT TG; SEQ ID NO. 93) and MunIBOTTOM (AAT TCA AAA ATA AAC ACG TTT AAA CAT AAC AC; SEQ ID NO. 94) were annealed and ligated into the unique MunI site of p5FloxHRF to generate plasmid pDV153. Insertion of the oligo destroyed the original MunI site by changing one base at its 5′ end, but resulted in insertion of a new MunI site that is 32 base pairs closer to the fiber ORF than the original MunI site.

Construction of an Ad vector with a Chimeric Ad5/Ad37 Fiber Gene

To replace the Ad5 fiber sequence of pDV153 with Ad37 fiber, pDV153 was digested with SphI and MunI. This removed all but the N-terminal 183 nucleotides of Ad5 fiber (see SEQ ID NO. 1). Ad37 fiber was then PCR amplified using a 5′ primer (F37 5′SphI) TAC CAA TGG CAT GCT ATC CCT CAA GG (SEQ ID NO. 95) that added a SphI site and a 3′ primer (F37 3′EcoRI) AAA CAC GGG AAT TCG TCT TTC ATT C (SEQ ID NO. 96) that added an EcoRI restriction site. The 3′ primer was designed to have an EcoRI site since the Ad37 fiber sequence contains a MunI restriction site. The nucleotide overhangs left by digestion with EcoRI and MunI are compatible, allowing the PCR products to be cloned into pDV153 digested with SphI and MunI. This resulted in expression of a chimeric Ad5/Ad37 fiber protein with the N-terminal 61 amino acids from Ad5 fiber (SEQ ID NO. 2) and the remainder of the protein from Ad37 (corresponding to amino acid 62 to the end of Ad37 fiber; SEQ ID NO. 32).

After Ad37 fiber was ligated into pDV153, the SpeI/PacI fragment was used to replace the SpeI/PacI fragment of pAdEasy, resulting in plasmid pDV158. Plasmid pDV158 was then recombined with the shuttle plasmid pAdTrack, which contains a CMV-driven EGFP reporter gene (He et al., Proc. Natl. Acad. Sci. USA 95:2509-2514 (1998); U.S. patent Ser. No. 5,922,576). The resulting Ad vector (Ad5.GFP.37F) has the EGFP reporter at the site of the E1 deletion and the chimeric Ad5/Ad37 fiber gene in the viral chromosome, and infects cells via the Ad37 receptor rather than CAR. pDV158 can be readily used to create adenovirus particles with the same fiber protein but different transgenes.

Propagation of Ad5.GFP.37F in 633 Cells

The Ad5.GFP.37F genome is infectious, and readily begins replicating as a virus. Since the 293 cells (ATCC Accession No. CRL 1573) normally used for Ad propagation do not express high levels of the Ad37 receptor, this virus does not efficiently propagate. To facilitate viral amplification, stocks of the virus were maintained in the 633 cell line (ATCC Accession No. PTA-1145), which expresses a wildtype Ad5 fiber protein (Von Seggern et al., J. Virol. 74:354-362 (2000)). The particles therefore contain the Ad5 fiber produced by the cells and the chimeric Ad5/Ad37 fiber protein encoded by the virus. The Ad5 fiber allows the virus to re-infect the cell lines used for viral growth. A final round of growth in 293 cells (which do not express a fiber protein) generates particles with only the vector-encoded Ad37 fiber. To assess Ad5 and Ad37 fiber content of Ad5.GFP.37F particles, viral particles produced in either 633 cells or 293 cells were immunoblotted with anti-fiber monoclonal Ab 4D2. 633-grown particles contained the Ad5 and Ad5/37 fibers, while virus produced in 293 cells contained only the Ad5/37 chimeric fiber. Particles of the first-generation Ad vector Ad5.βgal.wt, which contain only the wildtype Ad5 fiber (Wu et al., Virology 279:78-89 (2001)), were included as a positive control. As a loading control, the same blot was re-probed with a polyclonal antibody against the viral penton base protein.

Preparation of Additional Ad5 Genomes Encoding Heterologous Fibers

These same procedures can be used to construct Ad5 genomes containing the 19p (SEQ ID NO. 33), 16 (SEQ ID NO. 37), 30 (SEQ ID NO. 35) and 35 (SEQ ID NO. 39) fibers. To improve incorporation of the fiber in the resulting particle, each fiber was modified to include the N-terminal 61 amino acids of Ad5 (see SEQ ID NO. 2 or see nucleotides 1-183 in SEQ ID NO. 1) by replacing the corresponding amino acids (i.e., the first 61 amino acids) of each heterologous fiber. Similar constructs can be made with other heterologous fibers and genomes, such as Ad2.

For example, for construction of the Ad5/Ad 16 chimeric fiber vector, plasmid pDV153 was digested with SphI and MunI to remove all but the first 183 nucleotides of Ad5 fiber. Ad16 fiber (SEQ ID NO. 37) was PCR amplified using 5′ primer F16 5′ SphI: GCC AGC GGC ATG CTC CAA CTT AAA (SEQ ID NO. 97) and 3′ primer F16 3′ MunI: TTT ATC AAT TGT GTT GTC AGT CAT CTT C (SEQ ID NO. 98), which contained SphI and MunI sites, respectively. The PCR product was ligated with plasmid pDV153 to generate plasmid pDV182. This resulted in expression of a chimeric Ad5/Ad16 fiber protein with the N-terminal 61 amino acids from Ad5 fiber (SEQ ID NO. 2) and the remainder of the protein from Ad16 (corresponding to amino acid 62 to the end of Ad16 fiber; SEQ ID NO. 38).

### Example 20

Tripartite leader sequences (TPLs) that are useful in enhancing the expression of complementing adenoviral proteins, particularly fiber protein, for use in preparing an adenoviral gene delivery vector are provided. The complete Ad5 TPL was constructed by assembling PCR fragments. First, the third TPL exon (exon 3) (nt 9644-9731 of the Ad5 genome) was amplified from Ad5 genomic DNA using the synthetic oligonucleotide primers 5′CTCAACAATTGTGGATCCGTACTCC3′ (SEQ ID NO. 99) and 5′GTGCTCAGCAGATCTTGCGACTGTG3′ (SEQ ID NO. 100). The resulting product was cloned to the BamHI and BglII sites of pΔE1Sp1a (Microbix Biosystems; see also, U.S. Pat. No. 6,140,087 and U.S. Pat. No. 6,379,943) using sites in the primers (shown in bold) to create plasmid pDV52. A fragment corresponding to the first TPL exon (exon 1), the natural first intron (intron 1), and the second TPL exon (exon 2) (Ad5 nt 6049-7182) was then amplified using primers 5′GGCGCGTTCGGATCCACTCTCTTCC3′ (SEQ ID NO. 101) and 5′CTACATGCTAGGCAGATCTCGTTCGGAG3′ (SEQ ID NO. 102), and cloned into the BamHI site of pDV52 (again using sites in the primers) to create pDV55.

This plasmid contains a 1.2 kb BamHI/BglII fragment containing the first TPL exon, the natural first intron, and the fused second and third TPL exons. The nucleotide sequence of the complete TPL containing the noted 5′ and 3′ restriction sites is shown in SEQ ID NO. 103 with the following nucleotide regions identified: 1-6 nt BamHI site; 7-47 nt first leader segment (exon 1); 48-1068 nt natural first intron (intron 1); 1069-1140 nt second leader segment (exon 2); 1141-1146 nt fused BamHI and BglII sites; 1147-1234 nt third leader segment (exon 3); and 1235-1240 nt BglII site.

### Example 21

### Preparation of Adenoviral Gene Delivery Vectors Using Adenoviral Packaging Cell Lines

Adenoviral delivery vectors are prepared to separately lack the combinations of E1/fiber and E4/fiber. Such vectors are more replication-defective than those previously in use due to the absence of multiple viral genes. A preferred adenoviral delivery vector is replication competent but only via a non-fiber means is one that only lacks the fiber gene but contains the remaining functional adenoviral regulatory and structural genes. Furthermore, these adenovirus delivery vectors have a higher capacity for insertion of foreign DNA.

### A. Preparation of Adenoviral Gene Delivery Vectors Having Specific Gene Deletions and Methods of Use

To construct an E1/fiber deleted viral vector containing the LacZ reporter gene construct, two new plasmids were constructed. The plasmid pAE1Bβgal was constructed as follows. A DNA fragment containing the SV40 regulatory sequences and E. coli β-galactosidase gene was isolated from pSVβgal (Promega) by digesting with VspI, filling the overhanging ends by treatment with Klenow fragment of DNA polymerase I in the presence of dNTPs and digesting with BamHI. The resulting fragment was cloned into the EcoRV and BamHI sites in the polylinker of pΔE1sp1B (Microbix Biosystems; see also, U.S. Pat. No. 6,140,087 and U.S. Pat. No. 6,379,943) to form pΔE1Bβgal that therefore contained the left end of the adenovirus genome with the E1a region replaced by the LacZ cassette (nucleotides 6690 to 4151) of pSVβgal. Plasmid DNA may be prepared by the alkaline lysis method as described by Birnboim and Doly, Nuc. Acids Res., 7:1513-1523 (1978) or by the Quiagen method according to the manufacturer's instruction, from transformed cells used to expand the plasmid DNA. Plasmid DNA was then purified by CsCl-ethidium bromide density gradient centrifugation. Alternatively, plasmid DNAs may be purified from E. coli by standard methods known in the art (e.g. see Sambrook et al.)

The second plasmid (pDV44), prepared as described herein, is derived from pBHG10, a vector prepared as described by Bett et al., Proc. Natl. Acad. Sci., USA, 91:8802-8806 (1994) (see, also International PCT application No. WO 95/00655) using methods well known to one of skill in the art. This vector also is commercially available from Microbix Biosystems and contains an Ad5 genome with the packaging signals at the left end deleted and the E3 region (nucleotides 28133:30818) replaced by a linker with a unique site for the restriction enzyme PacI. An 11.9 kb BamHI fragment, which contains the right end of the adenovirus genome, is isolated from pBHG10 and cloned into the BamHI site of pBS/SK(+) to create plasmid p11.3 having approximately 14,658 bp. The p11.3 plasmid was then digested with PacI and SalI to remove the fiber, E4, and inverted terminal repeat (ITR) sequences.

This fragment was replaced with a 3.4 kb fragment containing the ITR segments and the E4 gene which was generated by PCR amplification from pBHG10 using the following oligonucleotide sequences: 5′ TGTACACCG GATCCGGCGCACACC3′ SEQ ID NO: 104; and 5′CACAACGAGCTC AATTAATTAATTGCCACATCCTC3′ SEQ ID NO: 105. These primers incorporated sites for PacI and BamHI. Cloning this fragment into the PacI and blunt ended SalI sites of the p11.3 backbone resulted in a substitution of the fused ITRs, E4 region and fiber gene present in pBHG10, by the ITRs and E4 region alone. The resulting p11.3 plasmid containing the ITR and E4 regions, designated plasmid pDV43a, was then digested with BamHI. This BamHI fragment was then used to replace a BamHI fragment in pBHG10 thereby creating pDV44 in a pBHG10 backbone.

In an alternative approach to preparing pDV44 with an additional subcloning step to facilitate the incorporation of restriction cloning sites, the following cloning procedure was performed. pDV44 as above was constructed by removing the fiber gene and some of the residual E3 sequences from pBHG10 (Microbix Biosystems; see, also U.S. Pat. No. 6,140,087). As above, to simplify manipulations, the 11.9 kb BamHI fragment including the rightmost part of the Ad5 genome was removed from pBHG10 and inserted into pBS/SK. The resulting plasmid was termed p11.3. The 3.4 kb DNA fragment corresponding to the E4 region and both ITRs of adenovirus type 5 was amplified as described above from pBHG10 using the oligonucleotides listed above and subcloned into the vector pCR2.1 (Invitrogen) to create pDV42. This step is the additional cloning step to facilitate the incorporation of a SalI restriction site. pDV42 was then digested with PacI, which cuts at a unique site (bold type) in one of the PCR primers, and with SalI, which cuts at a unique site in the pCR2.1 polylinker. This fragment was used to replace the corresponding PacI/XhoI fragment of p11.3 (the pBS polylinker adjacent to the Ad DNA fragment contains a unique XhoI site), creating pDV43. A plasmid designated pDV44 was constructed by replacing the 11.9 kb BamHI fragment of pBHG10 by the analogous BamHI fragment of pDV43. As generated in the first procedure, pDV44 therefore differs from pBHG10 by the deletion of Ad5 nucleotides 30819:32743 (residual E3 sequences and all but the 3′-most 41 nucleotides of the fiber open reading frame).

In summary, the cloning procedures described above result in the production of a fiber-deleted Ad5 genomic plasmid (pDV44) that was constructed by removing the fiber gene and some of the residual E3 sequences from pBHG10. pDV44 contains a wild-type E4 region, but only the last 41 nucleotides of the fiber ORF (this sequence was retained to avoid affecting expression of the adjacent E4 transcription unit). Plasmids pBHG10 and pDV44 contain unpackageable Ad5 genomes, and must be rescued by cotransfection and subsequent homologous recombination with DNA carrying functional packaging signals. In order to generate vectors marked with a reporter gene, either pDV44 or pBHG10 was cotransfected with pΔE1B1Bβgal, which contains the left end of the Ad5 genome with an SV40-driven β-galactosidase reporter gene inserted in place of the E1 region.

In general, and as described below, the method for virus production by recombination of plasmids followed by complementation in cell culture involves the isolation of recombinant viruses by cotransfection of any adenovirus packaging cell system, namely 211A, 211B, 211R, A549, Vero cells, and the like, with plasmids carrying sequences corresponding to viral gene delivery vectors.

A selected cell line is plated in dishes and cotransfected with pDV44 and pAE1Bβ gal using the calcium phosphate method as described by Bett et al., Proc. Natl. Acad. Sci., USA, 91:8802-8806 (1994). Recombination between the overlapping adenovirus sequences in the two plasmids leads to the creation of a full-length viral chromosome where pDV44 and pΔE1Bβgal recombine to form a recombinant adenovirus vector having multiple deletions. The deletion of E1 and of the fiber gene from the viral chromosome is compensated for by the sequences integrated into the packaging cell genome, and infectious virus particles are produced. The plaques thus generated are isolated and stocks of the recombinant virus are produced by standard methods.

Because of the fiber deletion, a pDV44-derived virus is replication-defective, and cells in which it is grown must complement this defect. The 211B cell line (a derivative of 293 cells which expresses the wild-type (wt) AD5 fiber and is equivalent to 211A on deposit with ATCC) was used for rescue and propagation of the virus described here. pDV44 and pAE1βgal were cotransfected into 211B cells, and the monolayers were observed for evidence of cytopathic effect (CPE). Briefly, for virus construction, cells were transfected with the indicated plasmids using the Gibco Calcium Phosphate Transfection system according to the manufacturer's instructions and observed daily for evidence of CPE.

One of a total of 58 transfected dishes showed evidence of spreading cell death at day 15. A crude freeze-thaw lysate was prepared from these cells and the resulting virus (termed Ad5.βgal.ΔF) was plaque purified twice and then expanded. To prepare purified viral preparations, cells were infected with the indicated Ad and observed for completion of CPE. Briefly, at day zero, 211B cells were plated in DMEM plus 10% fetal calf serum at approximately 1×10⁷ cells/150 cm² flask or equivalent density. At day one, the medium was replaced with one half the original volume of fresh DMEM containing the indicated Ad, in this case Ad5.βgal.ΔF, at approximately 100 particles/cell. At day two, an equal volume of medium was added to each flask and the cells were observed for CPE. Two to five days after infection, cells were collected and virus isolated by lysis via four rapid freeze-thaw cycles. Virus was then purified by centrifugation on preformed 15-40% CsCl gradients (111,000×g for three hours at 4° C.). The bands were harvested, dialyzed into storage buffer (10 mM Tris-pH 8.1, 0.9% NaCl, and 10% glycerol), aliquoted and stored at −70° C. Purified Ad5.βgal.ΔF virus particles containing human adenovirus Ad5.βgal.ΔFgenome (described further below) have been deposited with the ATCC on Jan. 15, 1999.

For viral titering, Ad preparations were titered by plaque assay on 211B cells. Cells were plated on polylysine-coated 6 well plates at 1.5×10⁶ cells/well. Duplicate dilutions of virus stock were added to the plates in 1 ml/well of complete DMEM. After a five hour incubation at 37° C., virus was removed and the wells overlaid with 2 ml of 0.6% low-melting agarose in Medium 199 (Gibco). An additional 1 ml of overlay was added at five day intervals.

As a control, the first-generation virus Ad5.9 gal.wt, which is identical to Ad5.βgal.ΔF except for the fiber deletion, was constructed by cotransfection of pBHG10 and pΔE1B1Bβgal. In contrast to the low efficiency of recovery of the fiberless genome ( 1/58 dishes), all of 9 dishes cotransfected with pΔE1Bβgal and pBHG10 produced virus.

In another embodiment, a delivery plasmid is prepared that does not require the above-described recombination events to prepare a viral vector having a fiber gene deletion. In one embodiment, a single delivery plasmid containing all the adenoviral genome necessary for packaging but lacking the fiber gene is prepared from plasmid pFG140 containing full-length Ad5 that is commercially available from Microbix. The resultant delivery plasmid referred to as pFG140-f is then used with pCLF (ATCC accession number 97737; and described in copending U.S. application Ser. No. 09/482,682 (also filed as International PCT application No. PCT/US00/00265 on Jan. 14, 2000)) stably integrated cells as described above to prepare a viral vector lacking fiber. For genetic therapy, the fiber gene can be replaced with a therapeutic gene of interest for preparing a therapeutic delivery adenoviral vector.

Vectors for the delivery of any desired gene and preferably a therapeutic gene are prepared by cloning the gene of interest into the multiple cloning sites in the polylinker of commercially available pΔE1sp1B (Microbix Biosystems; see also, U.S. Pat. No. 6,140,087), in an analogous manner as performed for preparing pE1Bβgal as described above. The same cotransfection and recombination procedure is then followed as described herein to obtain viral gene delivery vectors.

1. Characterization of the Ad5.βgal.ΔF Genome

To confirm that the vector genomes had the proper structures and that the fiber gene was absent from the Ad5.βgal.ΔF chromosome, the DNA isolated from viral particles was analyzed. Briefly, purified viral DNA was obtained by adding 10 μl of 10 mg/ml proteinase K, 40 μl of 0.5 M EDTA and 50 μl of 10% SDS to 800 μl of adenovirus-containing culture supernatant. The suspension was then incubated at 55° C. for 60 minutes. The solution was then extracted once with 400 μl of a 24:1 mixture of chloroform:isoamyl alcohol. The aqueous phase was then removed and precipitated with sodium acetate/ethanol. The pellet was washed once with 70% ethanol and lightly dried. The pellet was then suspended in 40 μl of 10 mM Tris-HCl, pH 8.0, 1 mM EDTA. Genomic DNA from Ad5.βgal.wt and Ad5.βgal.ΔF produced the expected restriction patterns following digestion with either EcoRI or with NdeI. Southern blotting, performed with standard methods, with labeled fiber DNA as a probe demonstrated the presence of fiber sequence in Ad5.βgal.wt but not in Ad5.βgal.ΔF DNA. As a positive control, the blot was stripped and reprobed with labeled E4 sequence. Fiber and E4 sequences were detected by using labeled inserts from pCLF and pE4/Hygro, respectively. E4 signal was readily detectable in both genomes at equal intensities. The complete nucleotide sequence of Ad5.βgal.ΔF is presented in SEQ ID NO: 106 and is contained in the virus particle deposited with ATCC.

2. Characterization of the Fiberless Adenovirus Ad5.βgal.ΔF

To verify that Ad5.βgal.ΔF was fiber-defective, 293 cells (which are permissive for growth of E1-deleted Ad vectors but do not express fiber) were infected with Ad5.βgal.ΔF or with Ad5.βgal.wt. Twenty-four hours post infection, the cells were stained with polyclonal antibodies directed either against fiber or against the penton base protein. Cells infected with either virus were stained by the anti-penton base antibody, while only cells infected with the Ad5.βgal.wt control virus reacted with the anti-fiber antibody. This confirms that the fiber-deleted Ad mutant does not direct the synthesis of fiber protein.

3. Growth of the Fiber-Deleted Ad5.βgal.ΔF Vector in Complementing Cells

Ad5.βgal.ΔF was found to readily be propagated in 211B cells. As assayed by protein concentration, CsCl-purified stocks of either Ad5.βgal.ΔF or Ad5.βgal.wt contained similar numbers of viral particles. The particles appeared to band normally on CsCl gradients. Infectivity of the Ad5.βgal.ΔF particles was lower than the Ad5.βgal.wt control, as indicated by an increased particle/PFU ratio. Ad5.βgal.ΔF was also found to plaque more slowly than the control virus. When plated on 211B cells, Ad5.βgal.wt plaques appeared within 5-7 days, while plaques of Ad5.βgal.ΔF continued to appear until as much as 15-18 days post infection. Despite their slower formation, the morphology of Ad5.βgal.ΔF plaques was essentially normal.

4. Production of Fiberless Ad5.βgal.ΔD Particles As Ad5.βgal.ΔF represents a true fiber null mutation and its stocks are free of helper virus, the fiber mutant phenotype was readily investigated. A single round of growth in cells (such as 293) which do not produce fiber generating a homogeneous preparation of fiberless Ad allowed for the determination of whether such particles would be stable and/or infectious. Either Ad5.βgal.wt or Ad5.βgal.ΔF was grown in 293 or 211B cells, and the resulting particles purified on CsCl gradients as previously described. Ad5.βgal.ΔF particles were readily produced in 293 cells at approximately the same level as the control virus and behaved similarly on the gradients, indicating that there was not a gross defect in morphogenesis of fiberless capsids.

Particles of either virus contained similar amounts of penton base regardless of the cell type in which they were grown. This demonstrated that fiber is not required for assembly of the penton base complex into virions. The Ad5.βgal.ΔF particles produced in 293 cells did not contain fiber protein. 211B-grown Ad5.βgal.ΔF also contained less fiber than the Ad5.βgal.wt control virus. The infectivities of the different viral preparations on epithelial cells correlated with the amount of fiber protein present. The fiberless Ad particles were several thousand-fold less infectious than the first-generation vector control on a per-particle basis, while infectivity of 211B-grown Ad5.βgal.ΔF was only 50-100 fold less than that of Ad5.βgal.wt. These studies confirmed fiber's crucial role in infection of epithelial cells via CAR binding.

5. Composition and Structure of the Fiberless Ad5.βgal.ΔF Particles

The proteins contained in particles of 293-grown Ad5.βgal.ΔF were compared to those in Ad5.βgal.wt, to determine whether proteolysis or particle assembly was defective in this fiber null mutant. The overall pattern of proteins in the fiberless particles was observed to be quite similar to that of a first-generation vector, with the exception of reduced intensity of the composite band resulting from proteins IIIa and IV (fiber). The fiberless particles also had a reduced level of protein VII. Although substantial amounts of uncleaved precursors to proteins VI, VII, and VIII were not seen, it is possible that the low-molecular weight bands migrating ahead of protein VII represent either aberrantly cleaved viral proteins or their breakdown products.

Cryo-electron microscopy was used to more closely examine the structure of the 293 grown Ad5.βgal.ΔF and of Ad5βgal.wt. The fiber, having an extended stalk with a knob at the end, was faintly visible in favorable orientations of wild-type Ad5 particles, but not in images of the fiberless particles. Filamentous material likely corresponding to free viral DNA was seen in micrographs of fiberless particles. This material was also present in micrographs of the first-generation control virus, albeit at much lower levels.

Three-dimensional image reconstructions of fiberless and wild-type particles at ˜20 Å resolution showed similar sizes and overall features, with the exception that fiberless particles lacked density corresponding to the fiber protein. The densities corresponding to other capsid proteins, including penton base and proteins IIIa, VI, and IX, were comparable in the two structures. This confirms that absence of fiber does not prevent assembly of these components into virions. The fiber was truncated in the wild-type structure as only the lower portion of its flexible shaft follows icosahedral symmetry. The RGD protrusions on the fiberless penton base were angled slightly inward relative to those of the wild-type structure. Another difference between the two penton base proteins was that there is a ˜30 Å diameter depression in the fiberless penton base around the five-fold axis where the fiber would normally sit. The Ad5 reconstructions confirm that capsid assembly, including addition of penton base to the vertices, is able to proceed in the complete absence of fiber.

6. Integrin-Dependent Infectivity of Fiberless Ad5.βgal.ΔF Particles

While attachment via the viral fiber protein is a critical step in the infection of epithelial cells, an alternative pathway for infection of certain hematopoietic cells has been described. In this case, penton base mediates binding to the cells (via β2 integrins) and internalization (through interaction with αv integrins). Particles lacking fiber might therefore be expected to be competent for infection of these cells, even though on a per-particle basis they are several thousand-fold less infectious than normal Ad vectors on epithelial cells.

To investigate this, THP-1 monocytic cells were infected with Ad5.βgal.wt or with Ad5.βgal.ΔF grown in the absence of fiber. Infection of THP-1 cells was assayed by infecting 2×10⁵ cells at the indicated m.o.i. in 0.5 ml of complete RPMI. Forty-eight hours post-infection, the cells were fixed with glutaraldehyde and stained with X-gal, and the percentage of stained cells was determined by light microscopy. The results of the infection assay showed that the fiberless particles were only a few-fold less infectious than first-generation Ad on THP-1 cells. Large differences were seen in plaquing efficiency on epithelial (211B) cells. Infection of THP-1 cells by either Ad5.βgal.ΔF or Ad5.βgal.wt was not blocked by an excess of soluble recombinant fiber protein, but could be inhibited by the addition of recombinant penton base). These results indicate that the fiberless Ad particles use a fiber-independent pathway to infect these cells. Furthermore, the lack of fiber protein did not prevent Ad5.βgalΔF from internalizing into the cells and delivering its genome to the nucleus, demonstrating that fiberless particles are properly assembled and are capable of uncoating.

The foregoing results with the recombinant viruses thus produced indicates that they can be used as gene delivery tools in cultured cells and in vivo. For example, for studies of the effectiveness and relative immunogenicity of multiply-deleted vectors, virus particles are produced by growth in packaging lines and are purified by CsCl gradient centrifugation. Following titering, virus particles are administered to mice via systemic or local injection or by aerosol delivery to lung. The LacZ reporter gene allows the number and type of cells which are successfully transduced to be evaluated. The duration of transgene expression is evaluated in order to determine the long-term effectiveness of treatment with multiply-deleted recombinant adenoviruses relative to the standard technologies which have been used in clinical trials to date. The immune response to the improved vectors described here is determined by assessing parameters such as inflammation, production of cytotoxic T lymphocytes directed against the vector, and the nature and magnitude of the antibody response directed against viral proteins.

Versions of the vectors which contain therapeutic genes such as CFTR for treatment of cystic fibrosis or tumor suppressor genes for cancer treatment are evaluated in the animal system for safety and efficiency of gene transfer and expression. Following this evaluation, they are used as experimental therapeutic agents in human clinical trials.

B. Retargeting of Adenoviral Gene Delivery Vectors by Producing Viral Particles Containing Different or Altered Fiber Proteins

As the specificity of adenovirus binding to target cells is largely determined by the fiber protein, viral particles that incorporate modified fiber proteins or fiber proteins from different adenoviral serotypes (pseudotyped vectors) have different specificities. Thus, the methods of expression of the native Ad5 fiber protein in adenovirus packaging cells as described above also is applicable to production of different fiber proteins.

Chimeric fiber proteins can be produced according to known methods (see, e.g., Stevenson et al. (1995) J. Virol., 69:2850-2857). Determinants for fiber receptor binding activity are located in the head domain of the fiber and an isolated head domain is capable of trimerization and binding to cellular receptors. The head domains of adenovirus type 3 (Ad3) and Ad5 were exchanged in order to produce chimeric fiber proteins. Similar constructs for encoding chimeric fiber proteins for use in the methods herein are contemplated. Thus, instead of using the intact Ad5 fiber-encoding construct (prepared above and in U.S. application Ser. No. 09/482,682) as a complementing viral vector in adenoviral packaging cells, the constructs described herein are used to transfect cells along with E4 and/or E1-encoding constructs.

Briefly, full-length Ad5 and Ad3 fiber genes were amplified from purified adenovirus genomic DNA as a template. The Ad5 and Ad3 nucleotide sequences are available with the respective GenBank Accession Numbers M18369 and M12411. Oligonucleotide primers are designed to amplify the entire coding sequence of the full-length fiber genes, starting from the start codon, ATG, and ending with the termination codon TAA. For cloning purposes, the 5′ and 3′ primers contain the respective restriction sites BamHI and NotI for cloning into pcDNA plasmid. PCR is performed as described above.

The resulting products are then used to construct chimeric fiber constructs by PCR gene overlap extension (Horton et al. (1990) BioTechniques, 8:525-535). The Ad5 fiber tail and shaft regions (5TS; the nucleotide region encoding amino acid residue positions 1 to 403) are connected to the Ad3 fiber head region (3H; the nucleotide region encoding amino acid residue positions 136 to 319) to form the 5TS3H fiber chimera. Conversely, the Ad3 fiber tail and shaft regions (3TS; the nucleotide region encoding amino acid residues positions 1 to 135) are connected to the Ad5 fiber head region (5H; the nucleotide region encoding the amino acid residue positions 404 to 581) to form the 3TS5H fiber chimera. The fusions are made at the conserved TLWT (SEQ ID NO: 46) sequence at the fiber shaft-head junction.

The resultant chimeric fiber PCR products are then digested with BamHI and NotI for separate directional ligation into a similarly digested pcDNA3.1. The TPL sequence is then subcloned into the BamHI for preparing an expression vector for subsequent transfection into 211 cells or into alternative packaging cell systems. The resultant chimeric fiber construct-containing adenoviral packaging cell lines are then used to complement adenoviral delivery vectors as previously described. Other fiber chimeric constructs are obtained with the various adenovirus serotypes using a similar approach.

In an alternative embodiment, the use of modified proteins including with modified epitopes (see, e.g., Michael et al. (1995) Gene Therapy, 2:660-668 and International PCT application Publication No. WO 95/26412, which describe the construction of a cell-type specific therapeutic viral vector having a new binding specificity incorporated into the virus concurrent with the destruction of the endogenous viral binding specificity). In particular, the authors described the production of an adenoviral vector encoding a gastrin releasing peptide (GRP) at the 3′ end of the coding sequence of the Ad5 fiber gene. The resulting fiber-GRP fusion protein was expressed and shown to assemble functional fiber trimers that were correctly transported to the nucleus of HeLa cells following synthesis.

Similar constructs are contemplated for use in the complementing adenoviral packaging cell systems for generating new adenoviral gene delivery vectors that are targetable, replication-deficient and less immunogenic. Heterologous ligands contemplated for use herein to redirect fiber specificity range from as few as 10 amino acids in size to large globular structures, some of which necessitate the addition of a spacer region so as to reduce or preclude steric hindrance of the heterologous ligand with the fiber or prevent trimerization of the fiber protein. The ligands are inserted at the end or within the linker region. Preferred ligands include those that target specific cell receptors or those that are used for coupling to other moieties such as biotin and avidin.

A preferred spacer includes a short 12 amino acid peptide linker composed of a series of serines and alanine flanked by a proline residue at each end using routine procedures known to those of skill in the art. The skilled artisan will be with the preparation of linkers to accomplish sufficient protein presentation and to alter the binding specificity of the fiber protein without compromising the cellular events that follow viral internalization. Moreover, within the context of this disclosure, preparation of modified fibers having ligands positioned internally within the fiber protein and at the carboxy terminus as described below are contemplated for use with the methods described herein.

The preparation of a fiber having a heterologous binding ligand is prepared essentially as described in the above-cited paper. Briefly, for the ligand of choice, site-directed mutagenesis is used to insert the coding sequence for a linker into the 3′ end of the Ad5 fiber construct in pCLF.

The 3′ or antisense or mutagenic oligonucleotide encodes a preferred linker sequence of ProSerAlaSerAlaSerAlaSerAlaProGlySer (SEQ ID NO: 107) followed by a unique restriction site and two stop codons, respectively, to allow the insertion of a coding sequence for a selected heterologous ligand and to ensure proper translation termination. Flanking this linker sequence, the mutagenic oligonucleotide contains sequences that overlap with the vector sequence and allow its incorporation into the construct. Following mutagenesis of the pCLF sequence adding the linker and stop codon sequences, a nucleotide sequence encoding a preselected ligand is obtained, linkers corresponding to the unique restriction site in the modified construct are attached and then the sequence is cloned into linearized corresponding restriction site. The resultant fiber-ligand construct is then used to transfect 211 or the alternative cell packaging systems previously described to produce complementing viral vector packaging systems.

In a further embodiment, intact fiber genes from different Ad serotypes are expressed by 211 cells or an alternative packaging system. A gene encoding the fiber protein of interest is first cloned to create a plasmid analogous to pCLF, and stable cell lines producing the fiber protein are generated as described above for Ad5 fiber. The adenovirus vector described which lacks the fiber gene is then propagated in the cell line producing the fiber protein relevant for the purpose at hand. As the only fiber gene present is the one in the packaging cells, the adenoviruses produced contain only the fiber protein of interest and therefore have the binding specificity conferred by the complementing protein. Such viral particles are used in studies such as those described above to determine their properties in experimental animal systems.

### Example 22

### Preparation and Use of Adenoviral Packaging Cell Lines Containing Plasmids Containing Alternative TPLs

Plasmids containing tripartite leaders (TPLs) have been constructed. The resulting plasmids that contain different selectable markers, such as neomycin and zeocin, were then used to prepare fiber-complementing stable cell lines for use as for preparing adenoviral vectors.

A. pDV60

Plasmid pDV60 was constructed by inserting the TPL cassette of SEQ ID NO. 88 into the BamHI site upstream of the Ad5 fiber gene in pcDNA3/Fiber, a neomycin selectable plasmid (see, e.g., U.S. application Ser. No. 09/482,682 (also filed as International PCT application No. PCT/US00/00265 on Jan. 14, 2000); see also Von Seggern et al. (1998) J. Gen Virol., 79: 1461-1468). The nucleotide sequence of pDV60 is listed in SEQ ID NO: 108. Plasmid pDV60 is available from the ATCC under accession number PTA-1144.

B. pDV61

To construct pDV61, an Asp718/NotI fragment containing the CMV promoter, partial Ad5 TPL, wildtype Ad5 fiber gene, and bovine growth hormone terminator was transferred from pCLF (ATCC accession number 97737; and described in copending U.S. application Ser. No. 09/482,682 (also filed as International PCT application No. PCT/US00/00265 on Jan. 14, 2000)), to a zeocin selectable cloning vector referred to as pcDNA3.1/Zeo (+) (commercially available from Invitrogen and for which the sequence is known).

C. pDV67

In an analogous process, pDV67 containing complete TPL was constructed by transferring an Asp 718/XbaI fragment from pDV60 into pcDNA3.1/Zeo(+) backbone. The nucleotide sequence of pDV67 is set forth in SEQ ID NO. 109. Plasmid pDV67 is available from the ATCC under accession number PTA-1145.

D. pDV69

To prepare pDV69 containing a modified fiber protein, the chimeric Ad3/Ad5 fiber gene was amplified from pGEM5TS3H (Stevenson et al. (1995) J. Virol., 69: 2850-2857) using the primers 5′ATGGGAT CAAGATGAAGCGCGCAAGACCG3′ (SEQ ID NO. 110) and 5′CACTATAGCGGCCGCATTCTCAGTCATCTT3′ (SEQ ID NO. 111), and cloned to the BamHI and NotI sites of pcDNA3.1/Zeo(+) via new BamHI and NotI sites engineered into the primers to create pDV68. Finally, the complete TPL fragment described above was then added to the unique BamHI site of pDV68 to create pDV69. The nucleotide sequence of pDV69 is listed in SEQ ID NO. 112 and the plasmid is available from the ATCC under accession number PTA-1146.

E. Preparation of Stable Adenovirus Packaging Cell Lines E1-2a S8 cells are derivatives of the A549 lung carcinoma line (ATCC # CCL 185) with chromosomal insertions of the plasmids pGRE5-2.E1 (also referred to as GRE5-E1-SV40-Hygro construct and listed in SEQ ID NO. 47) and pMNeoE2a-3.1 (also referred to as MMTV-E2a-SV40-Neo construct and listed in SEQ ID NO. 48), which provide complementation of the adenoviral E1 and E2a functions, respectively. This line and its derivatives were grown in Richter's modified medium (BioWhitaker)+10% FCS. E1-2a S8 cells were electroporated as previously described (Von Seggern et al. (1998) J. Gen Virol., 79: 1461-1468) with pDV61, pDV67, or with pDV69, and stable lines were selected with zeocin (600 μg/ml).

The cell line generated with pDV61 is designated 601. The cell line generated with pDV67 is designated 633 while that generated with pDV69 is designated 644. Candidate clones were evaluated by immunofluorescent staining with a polyclonal antibody raised against the Ad2 fiber. Lines expressing the highest level of fiber protein were further characterized.

For the S8 cell complementing cell lines, to induce E1 expression, 0.3 μM of dexamethasone was added to cell cultures 16-24 hours prior to challenge with virus for optimal growth kinetics. For preparing viral plaques, 5×10⁵ cells/well in 6 well plates are prepared and pre-induced with the same concentration of dexamethasone the day prior to infection with 0.5 μM included at a final concentration in the agar overlay after infection.

F. Cell Lines for Complementation of E1⁻/E2a⁻ Vectors

The Adenovirus 5 genome was digested with ScaI enzyme, separated on an agarose gel, and the 6,095 bp fragment containing the left end of the virus genome was isolated. The complete Adenovirus 5 genome is registered as Genbank accession #M73260 (or see SEQ ID NO. 1), incorporated herein by reference, and the virus is available from the American Type Culture Collection, Manassas, Va., U.S.A., under accession number VR-5. The ScaI 6,095 bp fragment was digested further with ClaI at bp 917 and BglII at bp 3,328. The resulting 2,411 bp ClaI to BglII fragment was purified from an agarose gel and ligated into the superlinker shuttle plasmid pSE280 (Invitrogen, San Diego, Calif.), which was digested with ClaI and BglII, to form pSE280-E.

Polymerase chain reaction (PCR) was performed to synthesize DNA encoding an XhoI and SalI restriction site contiguous with Adenovirus 5 DNA bp 552 through 924. The primers which were employed were as follows:

5′ end, Ad5 bp 552-585:

(SEQ ID NO. 113)

5′-GTCACTCGAGGACTCGGTC-GACTGAAAATGAGACATATTATCTGCC
ACGGACC-3′
3′ end, Ad5 bp 922-891:

(SEQ ID NO. 114)

5′-CGAGATCGATCACCTCCGGTACAAGGTTTGGCATAG-3′

This amplified DNA fragment (also referred to herein as Fragment A) was digested with XhoI and ClaI, which cleaves at the native ClaI site (bp 917), and ligated to the XhoI and ClaI sites of pSE280-E, thus reconstituting the 5′ end of the E1 region beginning 8 bp upstream of the ATG codon. PCR amplification then was performed to amplify Ad 5 DNA from bp 3,323 through 4,090 contiguous with an EcoRI restriction site. The primers employed were as follows:

5′ end, Ad5 bp 3323-3360:

(SEQ ID NO. 115)

5′-CATGAAGATCTGGAAGGTGCTGAGGTACGATGAGACC-3′;
and
3′ end, Ad5 bp 4090-4060:

(SEQ ID NO. 116)

5′-GCGACTTAAGCAGTCAGCTG-AGACAGCAAGACACTTGCTTGATCCA
AATCC-3′.

This amplified DNA fragment (also referred to herein as Fragment B) was digested with BglII, thereby cutting at the Adenovirus 5 BglII site (bp 3,382) and EcoRI, and ligated to the BglII and EcoRI sites of pSE280-AE to reconstruct the complete E1a and E1b region from Adenovirus 5 bp 552 through 4,090. The resulting plasmid is designated pSE280-E1.

A construct containing the intact E1a/b region under the control of the synthetic promoter GRE5 was prepared as follows. The intact E1a/b region was excised from pSE280-E1, which was modified previously to contain a BamHI site 3′ to the E1 gene, by digesting with XhoI and BamHI. The XhoI to BamHI fragment containing the E1a/b fragment was cloned into the unique XhoI and BamHI sites of pGRE5-2/EBV (U.S. Biochemicals, Cleveland, Ohio) to form pGRE5-E1).

Bacterial transformants containing the final construct were identified. Plasmid DNA was prepared and purified by banding in CsTFA prior to use for transfection of cells.

G. Construction of Plasmid Including Adenovirus 5 E2A Sequence

The Adenovirus 5 genome was digested with BamHI and SpeI, which cut at bp 21,562 and 27,080, respectively. Fragments were separated on an agarose gel and the 5,518 bp BamHI to SpeI fragment was isolated. The 5,518 bp BamHI to SpeI fragment was digested further with SmaI, which cuts at bp 23,912. The resulting 2,350 bp BamHI to SmaI fragment was purified from an agarose gel, and ligated into the superlinker shuttle plasmid pSE280, and digested with BamHI and SmaI to form pSE280-E2 BamHI-SmaI.

PCR then was performed to amplify Adenovirus 5 DNA from the SmaI site at bp 23,912 through 24,730 contiguous with NheI and EcoRI restriction sites. The primers which were employed were as follows:

5′ end, Ad5 bp 24,732-24,708:

(SEQ ID NO. 117)

5′-CACGAATTCGTCAGCGCTTCTCGTCGCGTCCAAGACCC-3′;
3′ end, Ad5 bp 23,912-23,934:

(SEQ ID NO. 118)

5′-CACCCCGGGGAGGCGGCGGCGACGGGGACGGG-3′

This amplified DNA fragment was digested with SmaI and EcoRI, and ligated to the SmaI and EcoRI sites of pSE280-E2 Bam-Sma to reconstruct the complete E2a region from Ad5 bp 24,730 through 21,562. The resulting construct is pSE280-E2a.

In order to convert the BamHI site at the 3′ end of E2a to a SalI site, the E2a region was excised from pSE280-E2a by cutting with BamHI and NheI, and recloned into the unique BamHI and NheI sites of pSE280. Subsequently, the E2a region was excised from this construction with NheI and SalI in order to clone into the NheI and SalI sites of the pMAMneo (Clonetech, Palo Alto, Calif.) multiple cloning site in a 5′ to 3′ orientation, respectively. The resulting construct is pMAMneo E2a.

Bacterial transformants containing the final pMAMneo-E2a were identified. Plasmid DNA was prepared and purified by banding in CsTFA. Circular plasmid DNA was linearized at the XmnI site within the ampicillin resistance gene of pMAMneo-E2a, and further purified by the phenol/chloroform extraction and ethanol precipitation prior to use for transfection of cells.

H. Transfection and Selection of Cells

In general, this process involved the sequential introduction, by calcium phosphate precipitation, or other means of DNA delivery, of two plasmid constructions each with a different viral gene, into a single tissue culture cell. The cells were transfected with a first construct and selected for expression of the associated drug resistance gene to establish stable integrants. Individual cell clones were established and assayed for function of the introduced viral gene. Appropriate candidate clones then were transfected with a second construct including a second viral gene and a second selectable marker. Transfected cells then were selected to establish stable integrants of the second construct, and cell clones were established. Cell clones were assayed for functional expression of both viral genes.

A549 (ATCC Accession No. CCL-185) were used for transfection. Appropriate selection conditions were established for G418 and hygromycin B by standard kill curve determination.

Transfection of A549 Cells with plasmids Including E1 and E2a Regions.

pMAMNeo-E2a was linearized with XmnI with the Amp^{R} gene, introduced into cells by transfection, and cells were selected for stable integration of this plasmid by G418 selection until drug resistant colonies arose. The clones were isolated and screened for E2a expression by staining for E2a protein with a polyclonal antiserum, and visualizing by immunofluorescence. E2a function was screened by complementation of the temperature-sensitive mutant Ad5ts125 virus which contains a temperature-sensitive mutation in the E2a gene. (Van Der Viet, et al., J. Virology, Vol. 15, pgs. 348-354 (1975)). Positive clones expressing the E2a gene were identified and used for transfection with the 7 kb EcoRV to Xmnl fragment from pGRE5-E1, which contains the GRE5 promoted E1a/b region plus the hygromycin^{R} gene. Cells were selected for hygromycin resistance and assayed for E1a/b expression by staining with a monoclonal antibody for the E1 protein (Oncogene Sciences, Uniondale, N.Y.). E1 function was assayed by ability to complement an E1-deleted vector. At this point, expression and function of E2a was verified as described above, thus establishing the expression of E1a/b and E2a in the positive cell clones.

A transfected A549 (A549 (ATCC Accession No. CCL-185)); cell line showed good E1a/b and E2a expression and was selected for further characterization. It was designated the S8 cell line.

I. Preparation of Adenoviral Vectors Containing Ad5.βgal.ΔF Genome in S8 Fiber-Complementing Cell Lines

To prepare adenoviral vectors containing Ad5.βgal.ΔF (Ad5.βgal.ΔF has been was deposited the ATCC under accession number VR2636) in S8 cells containing alternative forms of TPL for enhancing the expression of fiber proteins, the protocol as described in Example 21 for preparing Ad5.βgal.ΔF in 211B cells was followed with the exception of pretreatment with 0.3 μM dexamethasone for 24 hours as described above. Thus, viral particles with the wildtype Ad5 fiber protein on their surface and containing the fiberless Ad5.βgal.ΔF genome were produced in 633 cells. Particles produced in 644 cells also contained the fiberless Ad5.βgal.ΔF genome, but had the chimeric 5T3H fiber protein, with the Ad3 fiber knob, on their surface. These viral preparations can be used to target delivery of the Ad5.βgal.ΔF, Ad5.GFP.ΔF, or other similarly constructed fiberless genome with either wild-type or modified fibers.

### Example 23

### Enhanced Infectivity of Dendritic Cells by Pseudotyped Adenoviral Particles

Bone marrow-derived dendritic cells were generated by culture of bone marrow cells from female Balb/C mice with GM-CSF and IL-4 (Inaba et al. (1998) Isolation of dendritic cells in Current Protocols in Immunology, John Wiley & Sons, Inc. Philadelphia, 3.7.1-3.7.15). To confirm that the cultured cells expressed surface markers characteristic of dendritic cells, the cells were stained with fluorescently-conjugated antibodies directed against CD11c, CD80, and CD86 and analyzed by fluorescence-activated cell sorting (FACS) analysis. Antibodies against the dendritic cell markers CD11c, CD80 and CD86 are commercially available, such as from eBioscience.

The primary dendritic cell cultures were infected with 100,000 viral particles/cell of Ad5.GFP.ΔF pseudotyped with either Ad5, Ad16, Ad19p, Ad30, Ad35 or Ad37 fiber. The percent of cells positive for virus-induced GFP expression was determined by FACS analysis 48 hours after infection. All infections were performed in triplicate, and the mean ±standard deviation was determined.

In agreement with previous experiments, Ad5.GFP.ΔF pseudotyped with Ad5 fiber infected dendritic cells poorly with approximately 10% of cells positive for GFP expression, which is likely due to the lack of CAR expression on dendritic cells. In contrast, viruses carrying the Ad16, Ad19p, Ad30, Ad35 or Ad37 fiber proteins demonstrated enhanced infectivity of dendritic cells (approximately 49%, 46%, 37%, 26% and 50% of cells were GFP-positive), indicating that the fiber receptors for these serotypes are expressed on dendritic cells.

### Example 24

### Subgroup D Adenoviruses Demonstrate Selective Infectivity

Sequence and phylogenetic analysis of adenovirus fiber DNA and amino acid sequence suggests that subgroup B and subgroup D viruses bind different cellular receptors (Havenga et al. (2002) J. Virol. 76:4612-4620). In addition, while subgroup B viruses (such as Ad16, Ad35 and Ad50), are capable of infecting a wide variety of cancer cell lines and primary cells, including endothelial cells, smooth muscle cells, synoviocytes, fibroblasts, amniocytes, dendritic cells, bone marrow stroma cells, chondrocytes, myoblasts, melanocytes, follicle dermal papilla cells and hematopoietic stem cells (Havenga et al. (2002) J. Virol. 76:4612-4620), subgroup D viruses have a more selective tropism.

To determine whether select subgroup B (Ad3, Ad16 and Ad35) and subgroup D (Ad19p, Ad30 and Ad37) adenoviruses exhibit the same cellular tropism, a panel of cancer cell lines were tested for their capacity to support Ad gene delivery. The cell lines used were PC-3 cells, HepG2 cells, LNCaP cells and DU 145 cells. These cell lines are available from the ATCC under accession numbers CRL-1435, HB-8065, CRL-10995 and HTB-81, respectively.

Each cell line was infected with either 1000, 5000 or 10,000 particles per cell of Ad5.GFP.ΔF pseudotyped with Ad5 (subgroup C), Ad3, Ad16, Ad19p, Ad30, Ad35 or Ad37 fiber. After 48 hours, virus-directed GFP expression was determined by FACS analysis. For PC-3 cells infected with 1000 particles per cell, little to no GFP expression was detected in cells infected with viruses pseudotyped with subgroup D fibers Ad19p, Ad30 and Ad37. In contrast, GFP-expression was detected in approximately 40% of PC-3 cells infected with Ad16 and Ad35 fiber containing viruses. A similar pattern of GFP expression was found with cells infected at higher multiplicities of infection (MOIs). Approximately 80% of PC-3 cells infected with 5000 particles per cell of adenoviruses pseudotyped with Ad16 or Ad35 fiber were GFP positive, whereas only 2% of PC-3 cells were GFP positive when infected with Ad19p or Ad30 fiber pseudotyped viruses.

Similarly, in HepG2 cells, approximately 80% of cells were GFP-positive when infected with 5000 particles per cell of Ad16 or Ad35 fiber pseudotyped viruses, but less than 25% were GFP-positive when infected with either Ad19p or Ad30 fiber pseudotyped viruses. In addition, less than 10% of LNCaP cells were GFP-positive when infected with either 5000 or 10,000 particles per cell of Ad19p, Ad30 or Ad37 fiber containing adenoviruses, whereas Ad16 and Ad35 fiber directed GFP expression in approximately 65% of LNCaP cells. A similar pattern of infection was found in DU 145 cells. These results further demonstrate that subgroup B adenoviruses have a wider cellular tropism than subgroup D viruses and provides additional evidence that subgroup B and subgroup D adenoviruses use different receptors for cell binding and infection.

### Example 25

### Immunization with Adenovirus Particles Pseudotyped with Ad37 Fiber Results in T-Cell Stimulation

The following experiment was performed to determine whether immunization of mice with adenoviral particles pseudotyped with fiber protein from subgroup D adenovirus leads to stimulation of CD8+ T cells. Mice (eight in each experimental group, four in the vehicle (control) group) were immunized by subcutaneous injection with 1×10¹⁰ particles of either Ad5.GFP.WT (Ad5 particles pseudotyped with Ad5 fiber) or Ad5.GFP.F37 (Ad5 particles pseudotyped with Ad37 fiber). Four weeks following inoculation, spleens were harvested to quantitate stimulation of T cells by determining the number of IFN-γ-positive CD8+ T cells.

To determine the percentage of activated CD8+ T cells in immunized mice, spleens were isolated and mechanically disrupted. Following lysis of red blood cells, 1×10⁶ splenocytes were cultured for three hours in RPMI with 10% fetal calf serum and Golgiplug (BD Biosciences), in the presence or absence of 0.1 μg/ml EGFP epitope peptide HYLSTQSAL or the irrelevant OVA peptide (SIINFEKL) as a control. Cells were then stained with an APC-conjugated anti-CD8 antibody (eBioscience), fixed and permeabilized using the Cytofix/Cytoperm kit (BD Biosciences) and stained with a PE-conjugated antibody against IFN-γ. The cells were analyzed by fluorescence activated cell sorting (FACS) and the percentage of CD8+ cells positive for IFN-γ was determined ((number of CD8+ IFN-γ+ cells divided by the total number of CD8+ T cells)×100).

Immunization with adenovirus particles pseudotyped with either Ad5 fiber or Ad37 fiber led to stimulation of CD8+ T cells, as indicated by production of IFN-γ in these cells. These results indicate adenovirus particles with Ad37 fiber are excellent vaccine candidates due to their ability to stimulate CD8+ T cells while avoiding transduction of liver cells.

Since modifications will be apparent to those of skill in this art, it is intended that this invention be limited only by the scope of the appended claims.

What is claimed is:

1. An adenovirus particle, comprising a heterologous fiber or a portion thereof, whereby binding of the viral particle to dendritic cells is increased compared to a particle that expresses its native fiber, wherein:
the adenovirus (Ad) particle, except for the fiber, is from a subgroup C adenovirus; and
the fiber includes fiber from a subgroup D adenovirus for binding to dendritic cells, wherein the subgroup D adenovirus is selected from the group consisting of adenovirus serotype 8, 9, 10, 13, 15, 17, 19a, 19p, 20, 22-30, 32, 33, 36, 38, 39 and 42-49 or
the fiber comprises fiber from a subgroup B adenovirus for binding the virus to dendritic cells, wherein the subgroup B adenovirus is selected from the group consisting of adenovirus serotype 7, 11, 14, 21, 34 and 50.

2. A particle of claim 1, wherein:
the fiber is chimeric and comprises an N-terminal portion from a fiber of a subgroup C adenovirus; and
the N-terminal portion is sufficient to increase incorporation into the particle compared to in its absence.

3. The particle of claim 1, wherein the fiber is a chimeric fiber that includes a sufficient portion of a subgroup D adenovirus fiber to target dendritic cells.

4. The particle of claim 1, wherein the subgroup C virus is selected from the group consisting of adenovirus serotype 1, 2, 5, and 6.

5. The particle of claim 1, wherein the fiber is further modified to reduce any interaction with CAR.

6. The particle of claim 1, wherein the fiber is modified to reduce any interaction with heparin sulfate proteoglycans (HSP).

7. The particle of claim 1, wherein the capsid includes further modifications that alter interaction with aV integrin.

8. The particle of claim 1, wherein the adenovirus (Ad) particle, except for the fiber, is from a subgroup C adenovirus; and
the fiber is from Ad19p.

9. The particle of claim 8, wherein the Ad19p fiber comprises at least a sufficient number of amino acids set forth as SEQ ID NO. 34 to target the particle to dendritic cells.

10. The particle of claim 9, wherein the Ad19p fiber comprises at least a sufficient number of amino acids set forth as SEQ ID NO. 34 to target the particle to dendritic cells, but exhibits reduced binding to HSP compared to a subgroup C fiber.

11. The particle of claim 1, wherein the adenovirus (Ad) particle, except for the fiber, is from a subgroup C adenovirus; and
the fiber is from Ad30.

12. The particle of claim 11, wherein the Ad30 fiber comprises at least a sufficient number of amino acids set forth as SEQ ID NO. 36 to target the particle to dendritic cells.

13. The particle of claim 11, wherein the Ad30 fiber comprises at least a sufficient number of amino acids set forth as SEQ ID NO. 36 to target the particle to dendritic cells, but exhibits reduced binding to HSP compared to a subgroup C fiber.

14. The particle of claim 11, wherein the fiber is chimeric and includes a portion of a subgroup C adenovirus.

15. An adenovirus particle of claim 1, comprising a mutation in the aV integrin-binding region of the capsid, whereby binding to the integrin is eliminated or reduced.

16. The adenovirus particle of claim 8, wherein the Ad19p fiber is modified by replacing the N-terminal 15, 16 or 17 amino acids with the 15, 16 or 17 amino acids of an Ad2 or Ad5 fiber.

17. The adenovirus particle of claim 11, wherein the Ad30 fiber is modified by replacing the N-terminal 15, 16 or 17 amino acids with the 15, 16 or 17 amino acids of an Ad2 or Ad5 fiber.

18. The adenovirus particle of claim 5, wherein the CAR-binding region of the capsid that is modified is on a fiber knob.

19. The adenovirus particle of claim 18, wherein the fiber protein further comprises one or more further modifications that reduce or eliminate interaction of the resulting fiber with HSP.

20. The adenovirus particle of claim 19, wherein the capsid further comprising a ligand, whereby the particle binds to a receptor for the ligand.

21. The adenovirus particle of claim 20, wherein the ligand is included in the knob region of the fiber.

22. The adenovirus particle of claim 20, wherein the ligand is inserted into the fiber or it replaces a portion of the fiber.

23. A particle of claim 1, further comprising a heterologous nucleic acid in the genome thereof, wherein the heterologous nucleic acid encodes an antigen or a product that alters dendritic cell activity.

24. The particle of claim 23, wherein the antigen is a tumor antigen or an antigen from a pathogen.

25. An adenovirus particle, comprising a heterologous fiber or a portion thereof, whereby binding of the viral particle to heparin sulfate proteoglycans (HSP) is reduced or eliminated compared to a particle that expresses its native fiber, wherein:
the adenovirus (Ad) particle, except for the fiber, is from a subgroup C adenovirus; and
the fiber comprises fiber from Ad19p or Ad30, whereby HSP interaction is reduced.

26. A composition formulated for administration to a subject comprising a particle of claim 1.

27. A composition of claim 26 formulated for intramuscular, IV or parenteral administration.

28. A composition of claim 26 that is a vaccine.

29. An immunotherapeutic method, comprising administering a composition of claim 26 to a subject.

30. A method of delivering viral particles to dendritic cells, comprising:
contacting a composition with cells that comprise dendritic cells, whereby viral particles bind to dendritic cells, wherein the composition contains a viral particle of claim 1 or an adenovirus particle that comprises a fiber from Ad37 for targeting the particle to dendritic cells and the adenovirus (Ad) particle, except for the fiber, is from a subgroup C adenovirus; and
infusing the composition into a subject.

31. The method of claim 30, wherein the cells are removed from the subject prior to contacting.

32. The method of claim 30, wherein the cells comprise immune cells.

33. The method of claim 30, wherein the cells are bone marrow cells.

34. A nucleic acid molecule encoding a viral particle of claim 1.

35. The nucleic acid molecule of claim 34 that comprises an adenovirus vector.

36. The nucleic acid molecule of claim 34 further comprising heterologous nucleic acid.

37. A cell, comprising the nucleic acid molecule of claim 34.

38. The cell of claim 37 that is a dendritic cell.

39. A cell, comprising the nucleic acid molecule of claim 36.

40. The cell of claim 39 that is a dendritic cell.

41. A method of treatment, comprising administering a cell to a subject who has an immune cell disorder, cancer or an infection, wherein the cell is a cell of claim 38 or a dendritic cell containing an adenovirus particle that comprises a fiber from Ad37 for targeting the particle to dendritic cells and the adenovirus (Ad) particle, except for the fiber, is from a subgroup C adenovirus.

42. The method of claim 41, wherein the subject is infected with a pathogen, has a tumor, an inflammatory disorder, allergies, asthma or an autoimmune disease.

43. A method of targeting an adenovirus particle to dendritic cells, comprising replacing all or a portion of the native fiber of the adenovirus with an adenovirus subgroup D fiber or an adenovirus subgroup B fiber.

44. The method of claim 43, wherein:
the adenovirus (Ad) particle, except for the fiber, is from a subgroup C adenovirus; and
the subgroup D adenovirus is selected from the group consisting of adenovirus serotype 8, 9, 10, 13, 15, 17, 19a, 19p, 20, 22-30, 32, 33, 36, 37, 38, 39 and 42-49 and the subgroup B adenovirus is selected from the group consisting of adenovirus serotype 3, 7, 11, 14, 16, 21, 34, 35 and 50.

45. The method of claim 43, wherein the subgroup C adenovirus is selected from the group consisting of adenovirus serotype 1, 2, 5, and 6.

46. The method of claim 43, wherein the fiber is further modified to reduce any interaction with CAR.

47. The method of claim 46, wherein the fiber is further modified to reduce any interaction with heparin sulfate proteoglycans (HSP).

48. The method of claim 47, wherein the capsid includes further modifications that alter interaction with αᵥ integrin.
